(19) United States
(12) Patent Application Publication
(10) Pub. No.: US 2023/0304064 A1
(43) Pub. Date: Sep. 28, 2023

(54) AUTOMATED, DIGITAL DISPENSING PLATFORM FOR MICRODILUTION ANTIMICROBIAL SUSCEPTIBILITY TESTING

(71) Applicant: Beth Israel Deaconess Medical Center, Inc., Boston, MA (US)

(72) Inventors: James E. KIRBY, Weston, MA (US); Kenneth P. Smith, Jamaica Plain, MA (US)

(21) Appl. No.: 16/309,368

(22) PCT Filed: Jun. 1, 2017
(86) PCT No.: PCT/US2017/035428

§ 371 Date: Dec. 12, 2018

Related U.S. Application Data
(60) Provisional application No. 62/349,900, filed on Jun. 14, 2016.

(51) Int. Cl. C12Q 1/18; G01N 35/10

(52) U.S. Cl. CPC C12Q 1/18; G01N 35/1016; G01N 2035/1039

(57) ABSTRACT

Provided herein are systems, methods, and articles of manufacture for automated antimicrobial testing. In particular, the systems and methods provided herein are directed to automated antimicrobial susceptibility testing platforms and methods for identification of synergistic antimicrobials that are faster, more precise, and less expensive than gold standard susceptibility methodologies.

### CROSS-REFERENCE TO RELATED APPLICATIONS

This application claims the benefit of U.S. Application Serial No. 62/349,900, filed Jun. 14, 2016, which is incorporated by reference herein as if set forth in its entirety.

### STATEMENT REGARDING FEDERALLY SPONSORED RESEARCH OR DEVELOPMENT

This invention was made with government support under R21AI119114 and R21AI112694 awarded by the National Institutes of Health. The government has certain rights in the invention.

### BACKGROUND

The rapid emergence of antimicrobial resistance has challenged current susceptibility testing paradigms. Due to their complexity and labor intensiveness, gold standard susceptibility methodologies — manual broth macrodilution, manual broth microdilution, and agar dilution susceptibility testing — are rarely, if ever, performed by hospital-based clinical microbiology laboratories. These standard susceptibility methodologies require a large number of pipetting steps to create an antimicrobial doubling dilution series that can be tested for antimicrobial effects and minimal inhibitory concentration (MIC) determination. The time and manual labor required for this type of testing, even for a few isolates or drugs, is prohibitive due to the fast-paced workflow required in clinical laboratories. Therefore, hospital-based clinical laboratories tend to employ more facile alternatives to gold standard susceptibility methodologies.

Alternatives in standard clinical use include MIC testing with pre-formulated antimicrobial dilution panels, e.g., Sensititre (ThermoFisher, Waltham, MA), MicroScan (Beckman Coulter, Indianapolis, IN), BD Phoenix (BD Diagnostics, Franklin Lakes, NJ) or MIC surrogate methods such as the Vitek 2 (bioMerieux, Durham, NC). While such alternative methods generally work well for common bacterial pathogens and most antimicrobials, not all antimicrobials are available in MIC panel test formats. Moreover, non-MIC-based methods such as disk diffusion or E-test strips (bioMerieux) may be substituted for select antimicrobials not available in panel testing methods but have several important limitations. Specifically, non-MIC-based methods are not appropriate for all antimicrobials. For example, Clinical Laboratory Standards Institute (CLSI) no longer recognizes disk diffusion as an appropriate method to test for susceptibility to colistin, a large lipopeptide antibiotic increasingly used to treat multidrug-resistant Enterobacteriaceae (Tzouvelekis et al., European Society of Clinical Microbiology and Infectious Diseases 20:862-872 (2014); Tan et al., J. Antimicrobial Chemotherapy 58:864-67 (2006)); CLSI. Performance Standard for Antimicrobial Susceptibility Testing. 27th ed. CLSI Supplement M100. Wayne, PA: Clinical and Laboratory Standards Institute; 2017), and the drug is currently not available on any FDA-cleared, pre-formulated MIC panel. Furthermore, the accuracy of colistin E-test strips has been found to be suboptimal for the organisms most likely to be treated with this drug (Dafopoulou et al., Antimicrobial Agents and Chemotherapy 59:4625-30 (2015)). Therefore, the only reliable option available to most clinical microbiology laboratories is to send isolates to a “reference laboratory” for dilution MIC testing.

During the past decade, there has been a dramatic emergence of multidrug-resistant Enterobacteriaceae (Center for Disease Dynamics Economics and Policy. 2015. State of the World’s Antibiotics, 2015. CDDEP, Washington, DC). In a survey of short- and long-term acute care hospitals in the United States, 17.8% and 3.6% of Enterobacteriaceae causing central line bloodstream infections, catheter-associated urinary tract infections, and surgical site infections were extended-spectrum β-lactam resistant and carbapenem resistant, respectively (Weiner et al., MMWR 65:235-241 (2016)). Limited therapeutic options remain to treat these multidrug-resistant pathogens, and practical availability of remaining active agents may be further limited by associated drug toxicities or patient allergies. Accordingly, there remains a critical need to test antimicrobials not available in pre-made panels or supplementary FDA-cleared methods. Colistin is a prime example of a drug that is effective against >85% of carbapenem-resistant Enterobacteriaceae (Bradford et al., Antimicrobial Agents and Chemotherapy 60:1385-92 (2015)) but not available in FDA-cleared susceptibility panels.

After decades of stagnation in antimicrobial development, several new antimicrobials were recently approved by the FDA or are in clinical trials. Such recent development has been spurred on by several initiatives including the Qualified Infectious Disease Product Designation in the United States, which confers additional years of patent protection and more rapid FDA review (Food and Drug Administration. 2014. Guidance for Industry: Expedited Programs for Serious Conditions - Drugs and Biologics). Two recent examples include ceftazidime/avibactam (Zhanel et al., Drugs 73:159-177 (2013)) and ceftolozane/tazobactam (Zhanel et al., Drugs 74:31-51 (2014)). Interpretive guidelines for these drugs exist (FDA Prescribing Information), but ceftolozane/tazobactam is only approved for testing Enterobacteriaceae using dilution-based methods. Ceftazidime/avibactam disks for disk diffusion have recently become available, but are only approved for testing Enterobacteriacaeae and Pseudomonas.

There exists a significant antimicrobial testing gap where current methodologies have not kept pace with introduction of new drugs or increasing frequency of antibiotic resistance. As a result, most hospital-based clinical microbiology laboratories must rely on reference laboratories to perform dilution-based reference testing for these critical, potentially lifesaving antimicrobials, a process that can delay the availability of susceptibility results by an additional 4 to 6 days. In the face of multidrug resistant pathogens with unpredictable susceptibility profiles, such a delay is clearly unsatisfactory. Just as importantly, the inability to test newer agents at the site of care, and therefore to offer confidence in their efficacy in a timely manner, is likely to have a chilling effect on use of new antimicrobials and their development. Accordingly, there remains a critical need for improved systems, methods, and devices for rapid antimicrobial susceptibility testing (AST) of patient isolates using unpredictable susceptibility profiles.

### BRIEF SUMMARY

Provided herein are improved systems and methods for rapid antimicrobial susceptibility testing suitable for various applications including clinical uses and identifying synergistically acting antimicrobials.

In one aspect, provided herein is an automated system for microscopy-based antimicrobial susceptibility testing. The system can comprise or consist essentially of a dispensing unit configured for automated dispensing of one or more compositions to one or more locations on a well plate; a communication module configured to communicate with a data storage module comprising antimicrobial susceptibility protocol information; a programmable controller configured to control an operation of the dispensing unit based on protocol information received from the data storage module via the communication module; and a microscopy system for automated detection of antimicrobial susceptibility. The microscopy system can be integrated with at least one of the dispensing unit, the communication module, and the programmable controller. The system can further comprise a digital dispenser apparatus configured to dispense apportioned picoliter to microliter volumes of one or more compositions to one or more locations on the well plate. The dispensing unit can comprise a first cassette configured to store and digitally dispense a suspension of at least one kind of cell or microorganism in a culture medium to one or more locations on the well plate; and a second cassette configured to store and digitally dispense an antimicrobial agent to one or more locations for automated dispensing to one or more locations on the well plate. The at least one kind of cell or microorganism can be selected from the group consisting of a prokaryotic cell, eukaryotic cell, bacterial cell, animal cell, fungus cell, insect cell, plant cell, virus, virus-containing host cell, and archaebacterial cell. The at least one kind of cell or microorganism can be a bacterium. The well plate can comprise a plurality of locations comprising a biocompatible, solid or semi-solid cell culture substrate. The dispensing unit can comprise a first cassette configured to store a culture medium for automated dispensing to one or more locations on a well plate, where the culture medium comprises a biocompatible solidifying agent; whereby, upon dispensation to the one or more locations, the culture medium solidifies to form a solid or semi-solid culture substrate; a second cassette configured to store and digitally dispense an antimicrobial agent to the one or more locations; and a third cassette configured to store and digitally dispense a suspension of at least one kind of cell or microorganism in a culture medium to the one or more locations. The controller can be programmed to dispense a predetermined quantity of culture medium from the first cassette, a predetermined quantity of antimicrobial agent from the second cassette, or a predetermined quantity of cell of interest from the third cassette. The culture medium can be selected from the group consisting of balanced salt solutions, nutrient mixtures, basal media, complex media, serum free media, insect cell media, virus production media, serum, fetal bovine serum, serum replacements, antibiotics, antimycotics, blood components other than serum, nicotinamide adenine dinucleotide, hemin, hematin, pyridoxal; or Isovitalex; lysed horse blood, and lysed sheep blood, or any combination thereof. The culture medium can comprise a biocompatible solidifying agent; whereby, upon dispensation to the one or more locations, the culture medium solidifies to form a solid or semi-solid culture substrate. The biocompatible solidifying agent can be a nonionic triblock copolymer formed from polyoxypropylene (poly(propylene oxide) and polyoxyethylene (poly(ethylene oxide)). The biocompatible solidifying agent can be selected from the group consisting of poloxamer 188 and poloxamer 407. The at least one kind of cell or microorganism can be selected from the group consisting of a prokaryotic cell, eukaryotic cell, bacterial cell, animal cell, fungus cell, insect cell, plant cell, virus, virus-containing host cell, and archaebacterial cell. The at least one kind of cell or microorganism can be a bacterium.

In another aspect, provided herein is an automated system for microscopy-based antimicrobial susceptibility testing. The system can comprise or consist essentially of a dispensing unit configured to receive one or more pre-loaded cassettes comprising one or more cells, microorganisms, or antimicrobial agents, and configured for automated dispensing from the one or more pre-loaded cassettes to one or more locations on a well plate; a communication module configured to communicate with a data storage module comprising antimicrobial susceptibility protocol information; a programmable controller configured to control an operation of the dispensing unit based on protocol information received from the data storage module via the communication module; and a microscopy system for automated detection of antimicrobial susceptibility. The microscopy system for automated detection of antimicrobial susceptibility can be configured to obtain images of one or more locations on the well plate. The system can further comprise a programmable computing system configured for analysis of images obtained by the microscopy imaging system, wherein the programmable computing system and the microscopy imaging systems are in communication with each other. The programmable computing system can comprise at least one of a data acquisition module, a processing module, and an analysis module. The programmable computing system can be configured to analyze the images using a convolutional neural network trained to predict growth or inhibition for individual images. The microscopy system can be integrated with at least one of the dispensing unit, the communication module, and the programmable controller. The system can further comprise a digital dispenser apparatus configured to dispense apportioned picoliter to microliter volumes from the one or more pre-loaded cassettes to one or more locations on the well plate.

In another aspect, provided herein is a method of using a digital dispenser apparatus for antimicrobial susceptibility testing. The method can comprise or consist essentially of manually pipetting a composition into a digital dispenser apparatus configured to dispense apportioned picoliter to microliter volumes of the composition to one or more locations on a well plate. The composition can be selected from the group consisting of an antimicrobial agent, a suspension of at least one kind of cell or microorganism in a culture medium, and a culture medium. The culture medium can comprise a biocompatible solidifying agent; whereby, upon dispensation to the one or more locations, the culture medium solidifies to form a solid or semi-solid culture substrate. The biocompatible solidifying agent can be a nonionic triblock copolymer formed from polyoxypropylene (poly(propylene oxide) and polyoxyethylene (poly(ethylene oxide)). The biocompatible solidifying agent can be selected from the group consisting of poloxamer 188 and poloxamer 407.

In a further aspect, provided herein is an automated system for antimicrobial susceptibility testing. The system can comprise or consist essentially of a dispensing unit configured for automated dispensing of one or more compositions to one or more locations on a well plate; a communication module configured to communicate with a data storage module comprising antimicrobial susceptibility protocol information; a programmable controller configured to control an operation of the dispensing unit based on protocol information received from the data storage module via the communication module; and a means for detecting antimicrobial susceptibility. The means for detecting antimicrobial susceptibility can be a means for spectrophotometric detection, microscopic detection, or fluorescence-based detection. The means for detecting antimicrobial susceptibility can comprise a microscopy imaging system configured to obtain images of one or more locations on the well plate. The system can further comprise a programmable computing system configured for analysis of images obtained by the microscopy imaging system, wherein the programmable computing system and the microscopy imaging systems are in communication with each other. The programmable computing system can comprise at least one of a data acquisition module, a processing module, and an analysis module. The programmable computing system can be configured to analyze the images using a convolutional neural network trained to predict growth or inhibition for individual images. The means for detecting antimicrobial susceptibility can be integrated with at least one of the dispensing unit, the communication module, and the programmable controller. The system can further comprise a digital dispenser apparatus configured to dispense apportioned picoliter to microliter volumes of one or more compositions to one or more locations on the well plate. The dispensing unit can comprise a first cassette configured to store and digitally dispense a suspension of at least one kind of cell or microorganism in a culture medium to one or more locations on the well plate; and a second cassette configured to store and digitally dispense an antimicrobial agent to one or more locations for automated dispensing to one or more locations on the well plate.

In another aspect, provided herein is a method of using a digital dispenser apparatus for antimicrobial synergy testing. The method can comprise or consist essentially of (a) manually pipetting two or more compositions into a digital dispenser apparatus configured to dispense apportioned picoliter to microliter volumes of each composition to one or more locations on a well plate, wherein each of the two or more compositions comprises a different antimicrobial agent; (b) manually pipetting a suspension of at least one kind of cell or microorganism in a culture medium into a digital dispenser apparatus configured to dispense apportioned picoliter to microliter volumes of the suspension to the one or more locations on a well plate of step (a); (c) detecting susceptibility of the at least one kind of cell or microorganism to the microbial agents of the two or more compositions; and (d) calculating a minimal inhibitory concentration (MIC) for each antimicrobial agent and calculating a fractional inhibitory concentration index (FIC_{I}), wherein the antimicrobial agents exhibit synergy where the FIC_{I} is <0.5.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### BRIEF DESCRIPTION OF THE DRAWINGS

The present invention will be better understood and features, aspects, and advantages other than those set forth above will become apparent when consideration is given to the following detailed description thereof. Such detailed description makes reference to the following drawings, wherein:

FIG. 1 is a graph showing Log2 variance from modal MIC. Log2 differences shown represent the number of two-fold dilutions away from the modal MIC for all antimicrobials tested during the precision study. 99.3% of the D300 digital dispensing measurements (n=432) and 96.2% of the manual broth microdilution (BMD) measurements (n= 184) were within ±1 two-fold dilution of the modal MIC, respectively.

FIG. 2 is a series of images of major clinical pathogens grown in microwell aqueous poloxamer 407 surfaces following digital dispensation by the D300 digital dispensing system.

FIG. 3 is a graph of dispense volumes from D300 versus colony forming units as determined from dispensate.

FIG. 4 presents data from a macroscopic MIC assay. E. coli and antibiotics were automatically dispensed into single wells of a 384-well plate and grown for 24 hours. The MIC was defined as the lowest concentration of antimicrobial resulting in complete growth inhibition.

FIG. 5 presents representative images collected by automated microscopy. Organisms were dispensed using the D300 digital dispensing system and imaged prior to incubation. White arrows indicate locations of E. coli (left panel) or S. aureus (right panel) cells.

FIG. 6 presents microscopic assessment of E. coli following four hours of growth. Gram negative organisms often have characteristic shape changes that can be indicative of antimicrobial effects (e.g., central bulging and bacterial elongation). These characteristics can be scored for susceptibility and resistance of isolates to individual antimicrobials.

FIGS. 7A-7D present representative image analysis workflow.

FIG. 8 is a block diagram showing various functional components that may be employed in an embodiment.

FIG. 9 presents workflow for an exemplary method of the invention.

FIGS. 10A-10E demonstrate linearity of digital dispensing of Gram-negative bacterial pathogens. (A-E) Standardized bacterial suspensions of indicated bacterial species were prepared in 0.9% NaCl containing 0.3% polysorbate-20. Bacterial suspension were dispensing using different size droplet volumes using a HP D300 digital dispensing system into sterile media within 384-well plate wells. Nanoliter droplets dispense volume is plotted against colony forming unites recovered from microtiter plate wells. The number of viable bacterial introduced into the wells was quantified by plating well contents onto agar plates and counting colonies. The data points shown represent the mean and standard deviations (error bars) calculated from three independent experiments. Volume dispensed was highly correlated with the number of bacteria recovered.

FIGS. 11A-11B demonstrate geographic precision of bacterial dispensing. Staphylococcus aureus was dispensed using digital dispenser into the center of a representative well in a 384-well plate on top of a solidified poloxamer growth surface. After four hours of incubation, the well surface was imaged. Microcolonies (grape-like clusters of cocci) were observed in the center of each well, but not outside the geographic target zone. Representative images from a single well of a multi-well plate are shown: (A) bacterial microcolonies are visible within target zone, (B) bacterial colonies are not present outside of target zone.

FIG. 12 demonstrates microscopic quantitation of bacterial dispensing precision. Bacteria were dispensed using the HP D300 into the center of solid microwell surfaces. Microscopic imaging showed that the number of bacteria detected in the central field was reproducible across multiple experiments. Error bars represent the standard deviation of three independent experiments.

FIG. 13 demonstrates growth of E. coli after 2 hours in the presence of different concentrations of meropenem either at or below the MIC. At the MIC, cells show swelling characteristic of treatment with carbapenem antibiotics. Below the MIC cells are arranged as microcolonies, indicating robust growth.

FIG. 14 demonstrates the accuracy of growth calls using convolutional neural network analysis. A deep convolutional neural network was trained on 3202 images of bacteria growing for 4 hours. Accuracy of growth calls in a test set was approximately 90%, which supports the feasibility of automated classification of images collected by automated microscopy.

FIGS. 15A-15C demonstrate an inoculum effect using digitally dispensed antibiotics and bacteria. Each point on the graph indicates an MIC measured at the indicated bacterial density. The horizontal red line is the MIC determined by reference broth microdilution. The vertical red line represents the CLSI-recommended bacterial inoculum (5 × 10⁵ cfu/ml). (A) and (B) are presumptive ESBL producing clinical isolates. The strain with MIC = 2 µg mL⁻¹ (A) shows a pronounced inoculum effect. At low bacterial concentrations (below CLSI-recommended inoculum), MICs are markedly reduced. Correspondingly, MICs are markedly elevated with increasing bacterial density (above CLSI-recommended inoculum), ultimately exceeding our detection limit (>64 µg mL⁻¹) at the highest concentrations. In comparison, a strain with a lower MIC (0.5 µg mL⁻¹) (B) showed greater stability in MICs across a range of inoculum densities and a steep inoculum effect starting at a higher inoculum density. (C) is a CLSI-recommended quality control strain with no beta-lactamases. It showed no inoculum effect. Small variations are due to the inherent ±1 two-fold precision of the method used.

FIG. 16 is a graph representing a combinatorial activity spectrum. Percent of trials of indicated antimicrobial combinations demonstrating synergy (FIC_{I-MIN} ≤0.5) and clinically relevant synergy (FICs of both antibiotics at the FIC_{I-MIN} within the susceptible or intermediate category) against a collection of 5 K. pneumoniae and 5 E. coli CRE strains. CST, colistin; RIF, rifampin; MEM, meropenem; MIN, minocycline; GEN, gentamicin; CHL, chloramphenicol; LVX, levofloxacin. Filled circles identify combinations for which synergy testing against CRE has not previously been reported.

FIGS. 17A-17G illustrate an exemplary microscopy-based antimicrobial susceptibility testing (MAST) assay. The HP D300 digital dispenser (A) and disposable small volume T8+ (B, top) or large volume D4+ (B, bottom) cassettes are used for antibiotic and cell dispensing. (C) Solid surfaces are prepared in single wells of a 384-well plate using CAMHB solidified with poloxamer-407 (CAMHB-P), and varying size droplets of antimicrobial are digitally dispensed into each well creating (D) a doubling dilution series. (E) Bacteria are dispensed on top of the antibiotic-containing well surfaces and incubated at 35±2° C. for 2 hours. (F) Images of cells are collected by automated microscopy and (G) classified as “growth” or “inhibition” using a machine learning algorithm. Areas defined by the ConvNet as showing inhibition are highlighted with red overlay. The minimal inhibitory concentration (MIC) of antimicrobial is defined as the lowest concentration resulting in bacterial growth inhibition (F, third well from right, corresponding to G, third image from right).

FIGS. 18A-18B demonstrate representative cell densities immediately after digital dispensing. Standardized suspensions of (A) E. coli ATCC 25922 (average of 164 cells/field or 1.6 cells/1000 µm²) or (B) E. cloacae ATCC 13047 (average of 264 cells/field or 2.58 cells/1000 µm²) were dispensed onto solid microwell surfaces using the HP D300 digital dispensing system and visualized using a Zeiss Cell Observer microscope. Arrows indicate individual cells.

FIGS. 19A-19D demonstrate representative morphologies of inhibited E. coli ATCC 25922. Antibiotics and E. coli ATCC 25922 were dispensed into microwells and automatically imaged with a Zeiss Cell Observer microscope after2-hour incubation. Panels represent the central field of a microwell containing: (A) ciprofloxacin, (B) cefepime, (C) gentamicin, (D) meropenem at the MIC. Insets in A-C show close-up views of an individual cell. Inset in D shows a close-up of two cells.

FIG. 20 is a graphical representation of MAST MIC assay output for E. coli ATCC 25922. Each point in panels A-D represents ConvNet output (fraction of image crops with inhibition probability >0.5) from the adjacent image at the indicated antibiotic concentration. Overlay in images indicates areas where the ConvNet algorithm detected bacterial inhibition. Solid line represents a sigmoid fit to the ConvNet data. Dashed line represents the threshold that delineates growth (left-side points) and inhibition (right-side points) and which is the best predictor of the MIC on a per antibiotic basis determined using reference broth microdilution testing and a standard 16-20 hour incubation.

FIG. 21 is a block diagram showing various functional components that may be employed in an embodiment.

FIG. 22 is a block diagram showing various functional components that may be employed in an embodiment.

FIG. 23 presents workflow for an exemplary method of the invention.

FIG. 24 depicts a convolutional neural network (CNN) that was used in a study of an exemplary implementation of the invention.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

All publications, including but not limited to patents and patent applications, cited in this specification are herein incorporated by reference as though set forth in their entirety in the present application.

The invention provided herein is based at least in part on the inventors’ discovery of novel, automated, at-will broth microdilution susceptibility testing platform. In particular, it was discovered that inkjet printer technology could be modified to digitally dispense, directly from stock solutions into a well plate, the two-fold serial dilution series required for broth microdilution testing. It was further discovered that the digital dispensing technology would be combined with automated absorbance readings and data analysis to determine minimal inhibitory concentrations with improved speed, cost effectiveness, and reproducibility. The technology described herein will enable hospital-based clinical microbiology laboratories to perform at-will broth microdilution testing of antimicrobials and address a critical testing gap.

### Systems

Accordingly, in a first aspect, provided herein is an automated system for antimicrobial susceptibility testing. By a microorganism, such as a clinical isolate or infectious agent, being “susceptible” is meant that the microorganism, (for example, a Mycobacterium), is deleteriously affected by an antibiotic in such a manner that such clinical isolate or infectious agent is rendered incompetent, noninfectious or non-viable as understood in the art (Yao, J. D. C. et al., In: Murray, P. R. et al., eds. Manual of Clinical Microbiology, ASM Press, Washington, D.C. (1995) pp. 1281-1307 (incorporated herein by reference)). Susceptible, as used herein, is synonymous with “susceptibility.” When a microorganism, such as a clinical isolate or infectious agent, is determined to be susceptible to a given antibiotic, the antibiotic is said to have “activity” against, or be “active” against such isolate or infectious agent. The term “antimicrobial susceptibility” is also understood to be the concentration of the antimicrobial agent at which a given percentage of microbial (e.g., bacterial, viral) replication is inhibited (e.g., the IC₅₀ for an anti-microbial agent is the concentration at which 50% of microbial replication is inhibited). Thus, a decrease in microbial drug susceptibility is the hallmark that an organism has acquired mutations or resistance elements that confers the ability to resist the inhibitory effects of the antimicrobial agent. In the clinical context, microbial drug resistance is evidenced by the antimicrobial agent being less effective or no longer being clinically effective in a patient. By “susceptibility testing” is meant an in vitro assay whereby the susceptibility of a microorganism, such as a clinical isolate or an infectious agent, to a series of antimicrobial compounds is determined, as understood in the art.

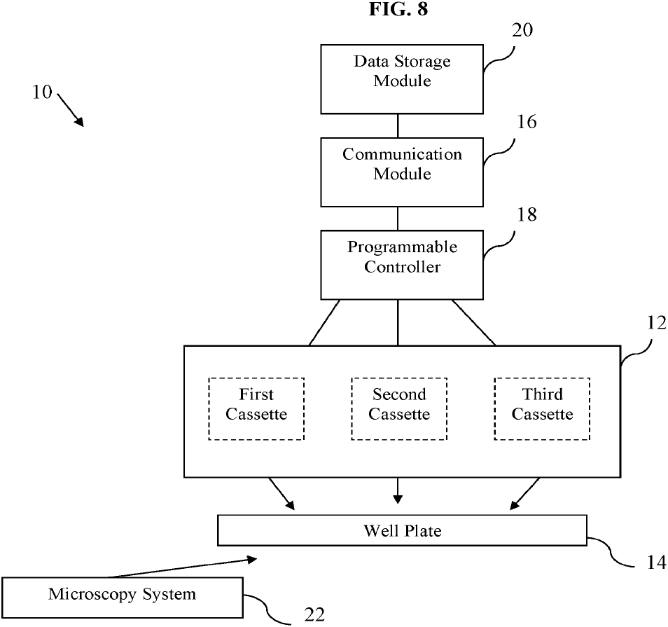

Referring to FIG. 8, automated system 10 can comprise dispensing unit 12 configured for automated dispensing of one or more compositions to one or more locations on well plate 14; a communication module 16 configured to communicate with a data storage module 20 comprising antimicrobial susceptibility protocol information; a programmable controller 18 configured to control an operation of dispensing unit 12 based on protocol information received from data storage module 20 via the communication module 18; and a microscopy system 22 for automated detection of antimicrobial susceptibility.

In some cases, dispensing unit 12 comprises a first cassette configured to store a cell culture medium for automated dispensing to one or more locations on a substrate, for example, on a well plate, wherein the cell culture medium comprises a biocompatible solidifying agent; whereby, upon dispensation to the one or more locations, the cell culture medium solidifies to form a cell culture substrate. The dispensing unit 12 can further comprise a second cassette configured to store and digitally dispense an antimicrobial agent to the one or more locations, and, in some cases, a third cassette configured to store and digitally dispense cells of interest in a liquid cell culture medium to the one or more locations. As used herein, the term “cassette” refers to a device comprising one or more dispense heads and configured to digitally dispense a liquid solution or suspension onto a substrate. In some cases, cassettes are configured to dispense liquid volumes from picoliters up to microliters directly onto or into microwells of an assay or array plate using inkjet technology (e.g., thermal droplet-on-demand or thermal inkjet printing technology) or any alternative droplet dispensing method. Alternative droplet dispensing methods include, without limitation, use of a piezoelectric element in the print chamber (e.g, piezoelectric droplet-on-demand or piezoelectric inkjet printing), sonic pulse/acoustic dispensing in which a sonic or acoustic pulse is applied to elicit dispensation of a droplet of a precise size; use of electrostatic forces to transfer microvolumes of specific size to a destination plate through a push pull mechanism; and use of solenoids to expel droplets of precise size. Other suitable methods for producing of appropriate size through use of controlled physical forces to drive a fluid of interest through a small orifice are known and available in the art.

In some cases, the cassette is a cartridge preloaded with a sample (e.g., antimicrobial agent, cells, culture medium) for dispensing. In other cases, the cassette is configured to receive such samples prior to use or between uses. Preferably, cassettes useful for the automated system provided herein are configured to hold or store liquids in, for example, a reservoir. As used herein, the terms “hold” and “store” mean that liquids for dispensation by the automated system can be retained in the cassette over an extended period of time (e.g., the cassette is pre-loaded or can be kept in cold storage, etc.) or that a selected amount of material dispensed via pipet can be held or added prior to use of the cassette. In some cases, two or more assays may be performed before a cassette needs to be replaced or refilled, thus cutting down assay preparation time.

The programmable controller 18 can be operably connected to the dispensing unit 12. In some cases, programmable controller 18 is programmed to dispense a predetermined quantity of cell culture medium from the first cassette, a predetermined quantity of antimicrobial agent from the second cassette, or a predetermined quantity of microbe of interest from the third cassette. Preferably, the controller 18 is operable to cause the dispensing of quantities of each of a plurality of compositions (e.g., liquids, suspensions, solutions) to each of a plurality of locations on well plate 14. For example, the controller 18 can be programmed by inputting protocol information, which can include dispensing parameters such as an amount of liquid to be dispensed, number of liquids to be dispensed, and a location on a well plate on which the liquid(s) are to be dispensed. The automated system 10 can further comprise a graphic user interface to allow a user to input, for example, a predetermined testing protocol.

Preferably, the automated system further comprises an integrated means for microscopic analysis of cells. As described in Example 1, the system can comprise a microscope or microscopy system 22 configured for automated image collection and analysis. For example, a microscope can be connected to a digital camera for automated imaging of locations on a well plate. In some cases, automated imaging can be conducted on the center of each location on a well plate using 20-50 z-slices of approximately 1 µm thickness. In such cases, each image represents approximately 43,500 µm². After collapsing z-slices into a single image per well, it is possible to distinguish individual bacteria under magnification (e.g., 40X magnification, 640X magnification) and to detect antimicrobial effects on the cells.

In some cases, the automated system further comprises a digital dispenser apparatus configured to receive liquid dispensing cassettes and to accurately apportion picoliter to microliter volumes to one or more locations on a substrate, for example, on a well plate. As used herein, the term “digital dispenser” refers to an apparatus that utilizes liquid dispense cassettes based on inkjet technology to accurately apportion picoliter to microliter doses of compounds into wells on a well plate. In operation, the digital dispenser is loaded with a cassette. Under software control, the digital dispenser dispenses predetermined amounts of the samples into the wells. These single use dispense heads virtually eliminate cross-contamination. In an exemplary embodiment, the digital dispenser is based on a thermal inkjet printer, available as HP D300, from Hewlett Packard, Inc.

Any appropriate composition can be dispensed according to the system provided herein. In some cases, the composition is a culture medium. As used herein, the terms “media,” “medium,” “broth,” “culture broth,” and the like all refer to a nutrient mixture suitable to culture a desired cell or microorganism. As used herein, the term “microorganism” refers to a member of one of following classes: bacteria, fungi, algae, and protozoa, and can also include, for purposes of the present disclosure, viruses, prions, or other pathogens. In various embodiments, bacteria, viruses, and in particular, human and animal pathogens, are evaluated. It will be understood by practitioners in the art that the exact composition of a growth medium will be dictated by the cell or microorganism type to be dispensed, cultured, and assayed. In particular embodiments, a culture medium can comprise one or more of water, proteins, amino acids, caesein hydrolysate, salts, lipids, carbohydrates, salts, minerals, and pH buffers. A culture medium may also contain extracts such as meat extract, yeast extract, tryptone, phytone, peptone, and malt extract. Exemplary cell culture media include, without limitation, balanced salt solutions, nutrient mixtures, basal media, complex media, serum free media, insect cell media, virus production media, serum, fetal bovine serum, serum replacements, antibiotics, antimycotics, blood components other than serum, supplements including but not limited to nicotinamide adenine dinucleotide, hemin, hematin, pyridoxal; or Isovitalex; and lysed horse or sheep blood, or any combination thereof. The culture medium can be a commercially available culture medium such as, for example, cation-adjusted Mueller-Hinton broth (available from Becton Dickinson and other suppliers); cation-adjusted Mueller-Hinton broth with 2.5-5% laked horse blood; cation-adjusted Mueller-Hinton broth supplemented with Isovitalex or equivalent; RPMI 1640 with 0.2% glucose; Hemophilus test medium broth; Brain heart infusion broth; and Middlebrook 7H9 Broth (for mycobacteria). In some cases, RPMI 1640 is adjusted to pH of 7.0 and buffered with 0.165 mol/L MOPS (3-[N-morpholino] propanesulfonic acid) for analysis of yeast.

The dispensed composition is a culture medium. In some cases, the culture medium is a liquid culture medium. In other cases, the culture medium comprises a biocompatible solidifying agent. When dispensed to one or more locations on a substrate such as a well plate, the cell culture medium solidifies to form a solid or semi-solid cell culture substrate. Exemplary biocompatible solidifying agents include, without limitation, nonionic block copolymers (also known as pluronics) formed from polyoxypropylene (poly(propylene oxide) and polyoxyethylene (poly(ethylene oxide)). For example, the biocompatible solidifying agent can be poloxamer 188 or poloxamer 407. Other suitable biocompatible solidifying agents include, without limitation, agar, agarose, methylcellulose, acacia, alginic acid, bentonite, Carbopols (carbomers), carboxymethyl cellulose, ethylcellulose, gelatin, hydroxyethyl cellulose, hydroxypropyl cellulose, magnesium aluminum silicate (Veegum®), methylcellulose, polyvinyl alcohol, sodium alginate, tragacanth, xanthan gum, phytagel, silicone based gelling agents (some of these are optically clear), polyacrylamide, polyethylene oxide, polyAMPS (2-Acrylamido-2-methylpropane sulfonic acid)-based hydrogels, polyvinylpyrrolidone, and hyaluronan.

In some cases, cassettes are configured to dispense one or more antimicrobial agents. As used herein, “antimicrobials” and “antimicrobial agents” include antibiotics (also termed antibacterial) and anti-fungal, anti-viral, and anti-parasitic agents. Also encompassed in the terms “antimicrobial” and “antimicrobial agents” are antimicrobial antibodies (e.g., antibodies that bind to and directly kill organisms or enhance their clearance during infection), antimicrobial peptides, phages, phage lysins (e.g., bacteriophage endolysins, which are phage-encoded peptidoglycan hydrolases able to cause lysis of cells such as bacteria), anti-virulence compounds (e.g., anti-toxins that interfere with bacterial disease progression by binding to target proteins produced during infection or anti-adhesins that interfere with bacteria binding to tissue), and other alternative class or non-standard agents developed as therapeutic agents for treating infections caused by one or more microbial organisms. Exemplary anti-virulence compounds are described by Totsika, Curr Med Chem. 2016 Feb; 6(1): 30-37. No current AST platforms are able to test these alternative or non-standard antimicrobial agents singly or in combination.

Exemplary classes of antimicrobial agents include, without limitation, aminoglycosides (e.g., gentamicin, tobramycin, amikacin, netilmicin, apramycin, spectinomycin), carbapenems (e.g., ertapenem, imipenem, meropenem, doripenem), first and second generation cephalosporins (e.g., cefazolin, cefuroxime), third and fourth generation cephalosporins (e.g., cefotaxime or ceftriaxone, ceftazidime, cefepime); cephalosporins β-lactamase inhibitor combinations (e.g. ceftazidime-avibactam, ceftolozane-tazobactam); fluoroquinolones (e.g., ciprofloxacin, moxafloxacin, levofloxacin), anti-MRSA cephalosporins (e.g., ceftaroline), glycopeptides (e.g., vancomycin), tetracyclines (e.g., tetracycline, doxycycline, minocycline), penicillins (e.g., ampicillin-sulbactam, amoxicillin-clavulanic acid, nafcillin, piperacillin/tazobactam), monobactams (e.g., aztreonam), macrolides and ketolides (e.g., azithromyin, clarithromycin); lincosamides (e.g., clindamyin); oxazolidinones (e.g., linezolid, tedizolid); glycylcyclines (e.g., tigecycline); antifolates (e.g., trimethoprim/sulfamethoxazole): nucleoside analogue inhibitors (e.g., azidothymidine); RNA polymerase inhibitors (e.g., rifampicin); anti-mycobacterial agents (e.g., isoniazide, pyrizinamide, ethambutol, capreomycin); polymycins (e.g., colistin, polymyxin B); lipoglycopeptides (e.g., oritavancin, telavancin and dalbavancin); phenicols (e.g., chloramphenicol), lipopeptides (e.g., daptomycin); antifungals (e.g., amphotericin; azoles such as fluconazole, posaconazole, voriconazole; and echinocandins such as caspofungin, micafungin; terbenafine; flucytosine); anti-viral agents (e.g., azidothymidine, lamivudine, acyclovir, ganciclovir, valganciclovir, cidofivir, efavirenz, oseltamivir, raltegravir, zanamivir, peramivir, adamantane antivirals (e.g., amantadine, rimantadine), foscarnet, brincidofovir, famciclovir, valacyclovir, neuraminidase inhibitors, protease inhibitors, integrase strand transfer inhibitors); antimicrobial peptides (e.g., POL7080, Polyphor, Ltd.); antimicrobial antibodies (e.g., Salvecin (AR-301), Aerumab, MEDI3902_Aerucin); phages (e.g., AB-SA01 from AmpliPhi); and lysins (e.g., CF-3101, Contract Corp.; N-Rephasin, Intron Biotechnology). Antimicrobial antibodies in clinical development are described in Pew Charitable Trusts, “A Scientific Roadmap for Antibiotic Discovery,” available at pewtrusts.org/~/media/assets/2016/05/ascientificroadmapforantibioticdiscovery.pdf on the World Wide Web.

In some cases, the dispensate comprises a cell or plurality of cells. In other cases, the dispensate comprises viruses or viral particles. Preferably, the dispensate comprises cells, viruses, or viral particles in a biological sample (e.g., blood, blood culture broth, urine, serum) or in a buffer or culture medium. Cells appropriate for automated dispensation include, without limitation, prokaryotic cells, eukaryotic cells, bacterial cells, animal cells, fungus cells, insect cells, plant cells, archaebacterial cells, and virus-containing host cells. As used herein, the term “virus” includes wild type viruses, killed, live attenuated, inactivated and recombinant viruses. It further includes virus-based products such as viral vectors, viral particles such as virus-like particles (VLPs), or nucleocapsids. “Virus-containing host cells” can be prokaryotic cells (e.g., bacteria) or eukaryotic cells (e.g., mammalian cells, human cell line) infected with a virus, viral particle, or virus-like particle. In some cases, dispensate comprises virus grown in tissue culture cells.

The automated system is useful for digitally dispensing a dispensate in precise amounts for, in some cases, automated testing of susceptibility of microorganisms or infectious agents to various antimicrobial agents. For the systems, methods, and compositions described herein, microorganisms and infectious agents include, without limitation, bacteria including mycobacteria, viruses, fungi, parasites, protozoa, and any other infectious microorganism. A human or animal patient having a disease caused by such a microorganism or infectious agent is said to have an “infection” caused by such an agent, or to be “infected with” such agent. An infectious agent that causes disease is said to be “pathogenic.” Bacteria that are typically not pathogenic, and part of the patient’s normal bacterial flora, are said to be saprophytic. Under some circumstances, such as when the patient is immune compromised or immune suppressed (e.g., being infected with HIV, or having AIDS complex, or after having undergone an organ transplant), such saprophytic microorganisms can cause infection.

Exemplary uses of the system provided herein is for assessing multidrug-resistant (MDR) or extensively-drug resistant (XDR) bacteria for susceptibility to particular antimicrobial agents. As used herein, “MDR” means bacteria that are resistant to more than one antimicrobial agent or more than one agent in a class or category of antimicrobial agents. Commonly, MDR bacteria are resistant to two, three, or more antimicrobial agents or classes of antimicrobial agents. As used herein, “XDR” refers to bacterial resistance to multiple antimicrobial agents (in some cases, defined as resistant to three or more antimicrobial agents), and possible resistance to all, or nearly all, approved antimicrobial agents or classes of antimicrobial agents. Gram-positive and Gram-negative bacteria are both affected by the emergence and rise of antimicrobial resistance. Accordingly, bacteria assessed according to the systems and methods provided herein are preferably those often responsible for healthcare-associated infections and prone to multidrug resistance. Such bacteria include, without limitation, Staphylococcus aureus, Enterococcus spp., Enterobacteriaceae (e.g., Escherichia coli, Enterobacter cloacae, Enterobacter aerogenes; Serratia marcesens, Klebsiella pneumoniae, Klebsiella oxytoca, Proteus mirabilis, Proteus vulgaris); Pseudomonas aeruginosa, and Acinetobacter spp., Mycobacterium tuberculosis, Streptococcus pneumoniae, Staphylococcus epidermidis, Hemophilus influenza; Helicobacter pylori, Salmonella typhimurium, Salmonella typhi, Salmonella paratyphi, E. coli H7:0157, Shigella spp., Neisseria gonorrhoeae, Neisseria meningiditis; anaerobic organisms such as Bacteriodes fragilis, Propionibacterium acnes, and Clostridium difficile; organisms of biothreat concern (e.g., Bacillus anthracis; Brucella abortus; Brucella melintensis; Brucella suis; Burkholderia mallei, Burkholderia pseudomallei; Francisella tularensis; Yersinia pestis). Microorganisms assessed according to the systems and methods provided herein also include, without limitation, mycobacteria such as Mycobacterium tuberculosis complex; M. avium-intracellulare complex; M. kansasii; and rapid-growing mycobacteria such as M. fortuitum; M. chelonae; M. abscessus. Also included are yeast such as Candida species Cryptococcus neoformans; and Cryptococcus gattii, and filamentous fungi such as Aspergillus fumigatus; and parasites such as Giardia lamblia and Entamoeba histolytica).

Microorganisms also include viruses. Viruses appropriate for automated dispensation as described herein include, without limitation, orthomyxoviruses, (e.g., influenza virus), paramyxoviruses (e.g., respiratory syncytial virus, mumps virus, measles virus), adenoviruses, rhinoviruses, coronaviruses, reoviruses, togaviruses (e.g., rubella virus), parvoviruses, poxviruses (e.g., variola virus, vaccinia virus), enteroviruses (e.g., poliovirus, coxsackievirus), hepatitis viruses (including A, B and C), herpes viruses (e.g., Herpes simplex virus, varicella-zoster virus, cytomegalovirus, Epstein-Barr virus), rotaviruses, flaviviruses (e.g., Zika virus, Yellow Fever virus, Dengue Fever viruses, Japanese encephalitis virus, St. Louis encephalitis virus, West Nile virus, tick-borne viruses), alphaviruses (Chikungunya virus), Norwalk viruses, hantavirus, arenavirus, rhabdovirus (e.g., rabies virus), retroviruses (including human immunodeficiency virus (HIV), HTLV I, and II), papovaviruses (e.g., papillomavirus), polyomaviruses, picornaviruses, and the like.

Any of a number of detection systems and/or methods that provide an ability to detect an attribute of a microorganism can be used in accordance with the systems and methods provided herein. Depending on the particular application, detection of antimicrobial susceptibility is performed in liquid medium or on a solid surface. In one embodiment, provided herein is an automated system for microscopy-based antimicrobial susceptibility testing, where the system comprises a dispensing unit configured to receive one or more pre-loaded cassettes comprising one or more microbial samples or antimicrobial agents, and configured for automated dispensing from the one or more pre-loaded cassettes to one or more locations on a well plate; a communication module configured to communicate with a data storage module comprising antimicrobial susceptibility protocol information; a programmable controller configured to control an operation of the dispensing unit based on protocol information received from the data storage module via the communication module; and a microscopy system for automated detection of antimicrobial susceptibility. In some cases, the microscopy system is integrated with at least one of the dispensing unit, the communication module, and the programmable controller. The automated system can further comprise a digital dispenser apparatus configured to the dispensing units to accurately apportion picoliter to microliter volumes from the one or more pre-loaded cassettes to one or more locations on the well plate.

In some cases, systems and methods that provide real-time or near real-time detection of antimicrobial susceptibility are used. These include brightfield imaging, darkfield imaging, phase contrast imaging, fluorescence imaging, upconverting phosphor imaging, chemiluminescence imaging, evanescent imaging, near infra-red detection, confocal microscopy in conjunction with scattering, surface plasmon resonance (“SPR”), atomic force microscopy, and the like. Likewise, various combinations of detection systems and/or methods may be used in parallel or in complementary fashion to detect one or more attributes of a microorganism in accordance with the present disclosure.

In other cases, spectroscopic methods are used to detect antimicrobial susceptibility. Spectroscopic methods that can be used to detect antimicrobial susceptibility include, without limitation, fluorescence spectroscopy, diffuse reflectance spectroscopy, infrared spectroscopy, terahertz spectroscopy, transmission and absorbance spectroscopy, Raman spectroscopy, including Surface Enhanced Raman Spectroscopy (“SERS”), Spatially Offset Raman spectroscopy (“SORS”), transmission Raman spectroscopy, and/or resonance Raman spectroscopy or any combination thereof. In some cases, spectrophotometric detection comprises analysis using a microplate reader at, for example absorbance at 600 nM. Fluorescent detection of bacterial growth and/or cytotoxicity can be detected using membrane binding dyes such as FM4-64 (ThermoFisher), or membrane permeable or membrane impermeable DNA binding dyes including but not limited to SYTOX Orange or SYTOX Green (ThermoFisher).

A variety of other microorganism detection systems and/or methods have been used to detect and/or determine values associated with antimicrobial susceptibility including, for example, optical density, nephelometry, densiometry, flow cytometry, capillary electrophoresis, analytical chemistry and indicator-based methods of metabolite detection, protein output, molecular diagnostics, quartz crystal microbalance, bioluminescence, microcantilever sensors, and asynchronous magnetic bead rotation, among others, and are also included within the various aspects and embodiments.

### Methods

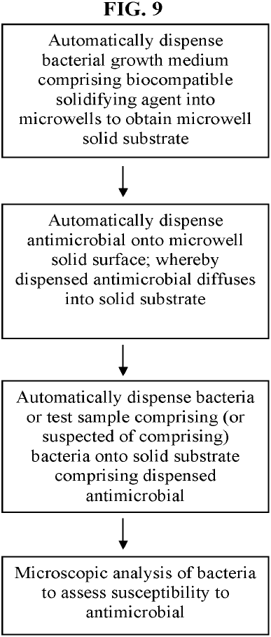

Antimicrobial susceptibility tests such as disk diffusion are not yet standardized or reliable for certain types of microorganisms, antimicrobials, (or combinations thereof), and therefore routine susceptibility tests cannot be performed in clinical laboratories. Accordingly, in another aspect, this disclosure provides improved methods for automating microscopy-based antimicrobial susceptibility testing. Referring to FIG. 9, the method can generally comprise digitally dispensed compounds, culture medium solutions, and cells directly onto a substrate (e.g., microwell culture plate) and analyzing (e.g., measuring and quantifying) the effects of candidate compounds on the cells. In some cases, the solutions are serial dilutions of antimicrobial agents. In exemplary embodiments, the methods include digitally dispensing one or more antimicrobials onto a cell culture substrate, where the antibiotic then diffuses through the cell culture substrate. In some cases, the method further comprises obtaining a dose-response curve. The effects of a test compound or compounds on cells, either individually or in combination, are analyzed using an automated microscope or plate reader. In some cases, concentrations of two or more antimicrobials may be varied independently. In other cases, concentrations of one or more antimicrobials may be varied and additional antimicrobials are added at fixed concentrations.

In certain embodiments, a method of using a digital dispenser apparatus for antimicrobial susceptibility testing comprises or consists essentially of manually pipetting a composition into a digital dispenser apparatus configured to dispense apportioned picoliter to microliter volumes of the composition to one or more locations on a well plate. Depending on the particular application of the method, the composition is an antimicrobial agent, a suspension of at least one kind of cell in a cell medium, or a cell culture medium, or a combination thereof. In some cases, the cell culture medium comprises a biocompatible solidifying agent as described herein, whereby, upon dispensation to the one or more locations, the cell culture medium solidifies to form a solid or semi-solid cell culture substrate.

Many antimicrobial compounds, including commercially available antimicrobials, cannot provide effective control of microorganisms, even at high use concentrations, due to weak activity against certain types of microorganisms, e.g., those resistant to some antimicrobial compounds. In search of more-effective chemotherapeutic approaches for treating infections, combination therapy is an important strategy, as synergistic interactions can potentially increase antimicrobial efficacy, reduce toxicity, cure faster, prevent the emergence of resistance, and provide broader-spectrum antimicrobial activity than monotherapy regimens. The use of synergistic combinations of drugs could have many advantages over conventional single compound chemotherapy, including lowered side-effects of drugs due to lower doses used or shorter time of chemotherapy; more rapid cure of infection, thus shortening hospital stays; increasing spectrum of pathogens controlled; decreasing incidence of development of resistance to antibiotics. A digital dispensing system or apparatus as described herein can be used to identify additional combinations of antimicrobial compounds having enhanced activity against various strains of microorganisms to provide effective control of the microorganisms. The methods provided herein are particularly advantageous for increasing the antibacterial potency against organisms that are resistant to broad-spectrum beta-lactam antibiotics, thus having utility for improved methods of preventing or treating bacterial infections in humans or animals.

In a further embodiment, therefore, provided herein is a method of using a digital dispensing apparatus to identify synergistic antimicrobial agents or compositions. Generally, synergy refers to two or more antimicrobial agents that exhibit greater antimicrobial activity when used in conjunction with each other than would be observed for the individual antimicrobial compounds. Quantitatively, synergism between two antimicrobial agents is indicated by a decrease in the minimum inhibitory concentration (MIC) of each test agent when used in combination, whereas antagonism is indicated by an increase in the MIC of either or both test agents when used in combination. As used herein, “synergy” is a fractional inhibitory concentration index (FIC_{I}) of ≦0.5, where FICI is defined as the sum of the fractional inhibitory concentrations (FICs) of the individual components in a combination of two compounds, and the FIC is defined as the ratio of the minimal inhibitory concentration (MIC) of the compound in the combination divided by the MIC of the compound alone. For example, to quantify the interactions between antibiotics being tested, the Fractional Inhibitory Concentration index (FIC_{I}) (i.e., the combination of antibiotics that produced the greatest change from an individual antibiotic’s MIC) value is calculated for each pathogen and antibiotic combination:

$$\frac{\text{A}}{{\text{MIC}}_{\text{A}}} + \frac{\text{B}}{{\text{MIC}}_{\text{B}}} = {\text{FIC}}_{\text{A}} + {\text{FIC}}_{\text{B}} = \text{FIC} \text{index} \left({\text{FIC}}_{\text{I}}\right)$$

where A and B are the MIC of each antibiotic in combination (in a single well), and MIC_{A} and MIC_{B} are the MIC of each drug individually. Accordingly, as used herein, “synergistic antimicrobial agents or compositions” refer to agents having a FIC_{I} ≦ 0.5. Any appropriate method for measuring a MIC can be used according to the methods described herein. Exemplary methods include, without limitation, automated microscopy, quantitative optical measurements (e.g., changes in optical properties of a cell suspension), observation of morphologic changes, and visual assessment of growth, or a combination of any of such methods. As described previously, automated microscopy provides a rapid way to determine the minimum inhibitory concentration (MIC) of test agents and resistance of the cells or microorganisms to test agents.

In certain embodiments, an in vitro method for testing synergy of two or more antimicrobial agents comprises or consists essentially of automated dispensing of apportioned picoliter to microliter volumes of test agents (e.g., an antibiotic or antimicrobial of interest), individually and in combination, in known concentrations in a culture medium. In some cases, test agents are automatically dispensed in known serial dilutions (e.g., serial two-fold dilutions). A suspension of the cell or microorganism to be tested is dispensed automatically onto the test agents in culture medium, and the cells or microorganisms of the suspension are incubated in the presence of the individual or combined test agents. The incubation can occur for a predetermined length of time. After incubation, the minimum inhibitory concentration (MIC) of each test agent used individually and in combination is determined, where the MIC is the lowest concentration of the test agent that inhibits growth in the medium, and then the fractional inhibitory concentration index (FIC_{I}) is calculated. Synergism is indicated by a FIC_{I} of ≦0.5. Generally, synergism is indicated by a decrease in the MIC of each test agent when used in combination, whereas antagonism is indicated by an increase in the MIC of either or both test agents when used in combination.

In certain embodiments, the in vitro method for antimicrobial synergy testing comprises or consists essentially of (a) manually pipetting two or more compositions into a digital dispenser apparatus configured to dispense apportioned picoliter to microliter volumes of each composition to one or more locations on a well plate, wherein each of the two or more compositions comprises a different antimicrobial agent; (b) manually pipetting a suspension of at least one kind of cell or microorganism in a culture medium into a digital dispenser apparatus configured to dispense apportioned picoliter to microliter volumes of the suspension to the one or more locations on a well plate of step (a); and (c) detecting susceptibility of the at least one kind of cell or microorganism to the microbial agents of the two or more compositions; (d) calculating a minimal inhibitory concentration (MIC) for each antimicrobial agent and calculating a fractional inhibitory concentration index (FIC_{I}), wherein the antimicrobial agents exhibit synergy where the FIC_{I} is ≤0.5. In some cases, the method further comprises (e) determining whether the concentration of each antibiotic in the synergistic combination is clinically relevant in that the concentration for each antimicrobial in the combination falls into a range that predicts activity with clinically achievable drugs levels.

In some cases, the in vitro method for antimicrobial susceptibility testing is a method of systematically testing antibiotic combinations for evidence of synergistic activity against a collection of carbapeneni-resistant microorganisms such as carbapenem-resistant Enterobacteriaceae (CRE) isolates. By applying the digital dispensing methods described herein to analysis of synergistic activity of certain antimicrobials against carbapenem-resistant microorganisms, it is possible to reduce or eliminate the problem of cumulative error in serial dilution and to reduce the time required to perform a checkerboard array. As described in Example 5, a checkerboard array can be set up in approximately 2 minutes, which includes manually pipetting stock antimicrobial solutions (one for each antibiotic) into, for example, a dispensing unit configured for automated dispensing of one or more compositions to one or more locations on a well plate and digitally dispensing such antimicrobials using pre-programmed protocols. In contrast, a synergy array prepared manually according to the protocol published by the American Society for Microbiology (Humphries RM. Testing: Broth Microdilution Checkerboard and Broth Macrodilution Methods. In: Leber AL, ed. Clinical Microbiology Procedures Handbook. Washington, D.C.: American Society of Microbiology, 2016; 5.16.1-5.8.23) involves the use of 8 different stock solutions (up to 5 per antibiotic depending on the concentration range to be tested), preparation of 18 intermediate antimicrobial concentrations, dispensing of each of these intermediate concentrations into an individual row or column of a microtiter plate, and addition of extra liquid media to reach final appropriate volumes, a process requiring 30 minutes at minimum.

It is understood by practitioners in the art that the MIC of some antimicrobial agents may be inoculum dependent. The inoculum effect as described by Chapman & Steigbiegel, J. Infect .Dis. 147:156 163 (1975) is evidenced by reduced activity of an antimicrobial agent in the presence of rapid proliferation of the microbe and/or high microbial load. For example, the minimum inhibitory concentration (MIC) for agents against Pseudomonas aeruginosa increases with increasing inoculum density (Eng, R. K., et al., Antimicrob. Ag. Chemother. 26:42-47 (1984)). Since the concentration of bacteria or other infectious agents responsible for a particular infection can vary depending on the site of the infection, the immune state of the infected subject, and on the stage of the infection, improved methods for detecting an inoculum effect on antimicrobial susceptibility are needed.

Inoculum effect experiments comprise a series of broth microdilution experiments in which the cell density of the bacterial inoculum is varied (often 10-fold) across multiple identical doubling dilutions of antibiotics. The MICs are then interpreted at each inoculum concentration. An organism is considered to demonstrate an inoculum effect when the measured MICs increase corresponding to the number of cells in the assay (inoculum density).

In another aspect, therefore, provided herein are methods for detecting an inoculum effect on antimicrobial susceptibility using automated/digital dispensing technology. In some cases, the method comprises using digital dispensing technology to dispense known concentrations of a bacterial inoculum. For example, by varying the droplet size during digital printing of a bacterial suspension as described herein, a desired inoculum of bacteria can be added to each testing well. More specifically, doubling dilutions of bacterial inocula can be performed, thereby establishing the relationship between inoculum and MIC through an inoculum dose-response curve that is much finer than the typical 10-fold dilution series as standardly performed.

One application of the method for detecting an inoculum effect is to screen for potential therapeutic agents for efficacy in suppressing resistance acquisition in one or more microbial cell populations. Another application of the inoculum effect testing methods includes, for example, predicting the likelihood of a microbial population of cells associated with a pathophysiological condition of acquiring resistance to a therapeutic agent due to an inoculum effect. These methods are further described and demonstrated in Example 4.

In certain embodiments, the systems and methods provided herein further comprise analyzing data collected by microscopy-based AST (MAST) using machine learning techniques. As used herein, the term “machine learning” refers to the use of algorithms to parse data, learn from it, and then make a determination or prediction based on representations in the data. Machine learning techniques have been used to analyze and learn from large data sets for a variety of applications. For example, machine learning techniques are useful for automating feature learning, image assessment, and image classification. In some cases, the machine learning technique is a deep learning technique. As used herein, “deep learning” (also known as deep structured learning, hierarchical learning or deep machine learning) is a form of machine learning in which multiple artificial networks containing multiple hidden layers of learned features or variables are used to evaluate and learn representations of data with multiple levels of abstraction.

In some cases, the machine learning technique employs a neural network. In such cases, therefore, the systems and methods provided herein further comprise implementing a neural network that is trained to learn and classify image features for a set of images collected by MAST. Neural networks are artificial networks of simple, connected processors called neurons, where each neuron produces a sequence of activations based on input data. Neural networks appropriate for the systems and methods described herein include, without limitation, deep neural networks, convolutional neural networks, fused convolutional neural networks, time convolutional neural networks, time-frequency convolutional neural networks, and/or any other suitable neural networks. In some embodiments, the neural network is a convolutional neural network. As used herein, the term “convolutional neural network” (also known as convnet or ConvNet) refers to an artificial, multilayered network having network architecture that includes at least one convolutional layer. Convolutional neural networks are known in the art. In some cases, the convolutional neural network (ConvNet) can be based on the VGG architecture (Simonyan & Zisserman, Very deep convolutional networks for large-scale image recognition. ICLR 2015, available at arxiv.org/abs/1409.1556 on the World Wide Web).

As described in the Examples that follow, single images and/or a series of images (e.g., a Z-series) can be collected from individual microwells of a particular experiment and analyzed for features of interest using, for example, using a VGG architecture-based convolutional neural network. As demonstrated in Example 6, a large set of training images can be used to train and validate the ConvNet to obtain a deep neural network capable of predicting growth or inhibition based on image features. Performance of the network (e.g., using MIC probabilities) can be determined by reference to broth microdilution (BMD) results for each dilution series. The Examples demonstrate that images can be classified with 80-90% or more accuracy on a per-image-crop basis, thus providing evidence of feasibility for automated classification of MAST images. Integrating automated microscopic imaging of bacterial replication with a deep learning approach for automated image classification enables rapid determination of antimicrobial minimal inhibitory concentrations at early time points with sufficient quality for machine learning classification.

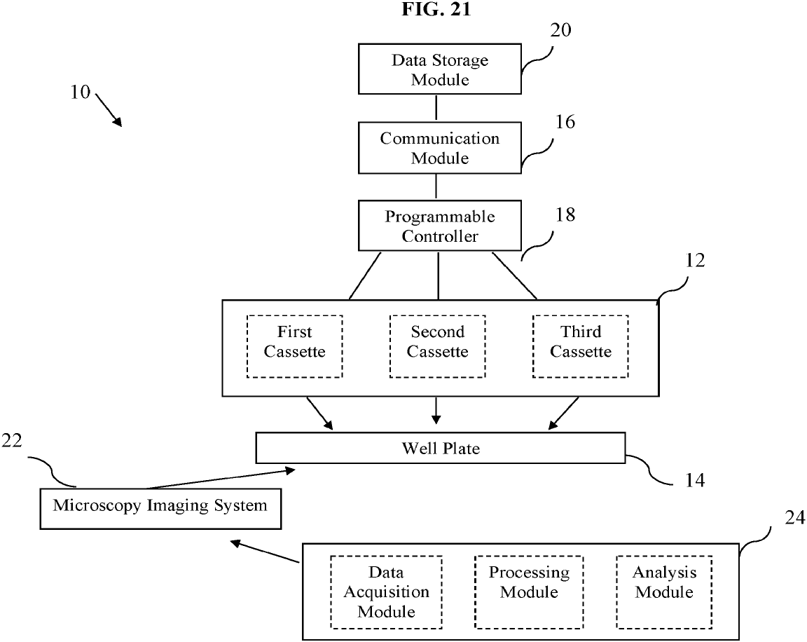

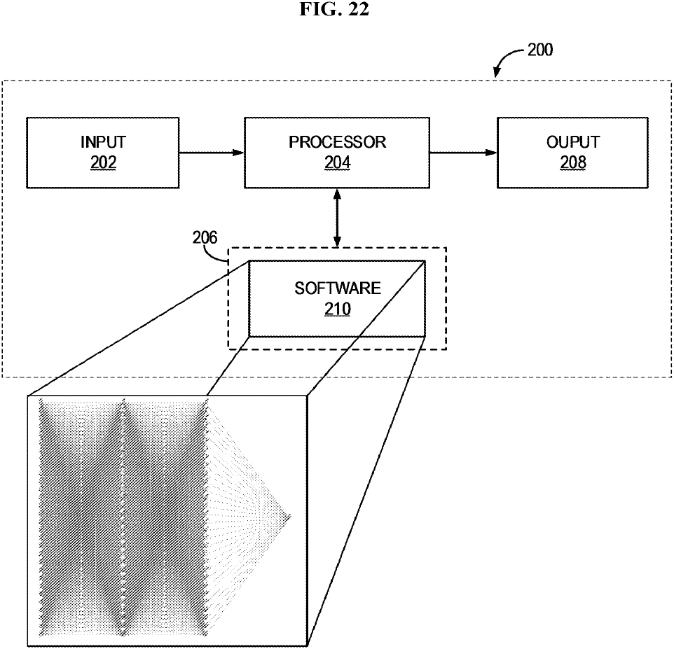

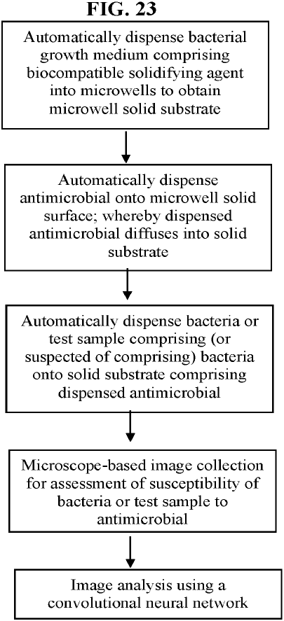

In certain embodiments, a system for automated dispensing and MAST can further comprise a computing device or other operating environment for implementing embodiments as described herein. Referring to FIG. 21, in some cases, a system for automated system for microscopy-based antimicrobial susceptibility testing as described further comprises one or more modules such as data acquisition, processing, and/or analysis modules for receiving, processing, and/or storing image data derived from an imaging system (e.g., a microscope-based imaging system). Such modules can be part of programmed computing system such as computing device 24. In some cases, the programming computing system is configured to perform one or more computer-assisted data operations (e.g., operating algorithms) for data manipulation and/or data analysis. In some cases, the data acquisition, processing, and/or analysis modules of computer device 24 are linked through a communications network.

Referring now to FIG. 22, a block diagram is illustrated of an example computer system 200 that can be used to implement the network processing, including neural network, in accordance with some aspects of the present disclosure. The system 200 generally may include an input 202, at least one processor 204, a memory 206, and an output 208. The system 200 may be, for example, a workstation, a notebook computer, a personal computing device or phone, a multimedia device or tablet, a network server, a mainframe, or any other general-purpose or application-specific computing device. In some configurations, the computer system 200 may form a part of a microscopy-based antimicrobial susceptibility testing (MAST) assay system or digital dispenser, such as described above. The computer system 200 may operate autonomously or semi-autonomously, or may read executable software instructions from a computer-readable medium (such as a hard drive, a CD-ROM, flash memory, and the like), or may receive instructions from a user, or any another source logically connected to a computer or device, such as another networked computer or server, via the input 202.

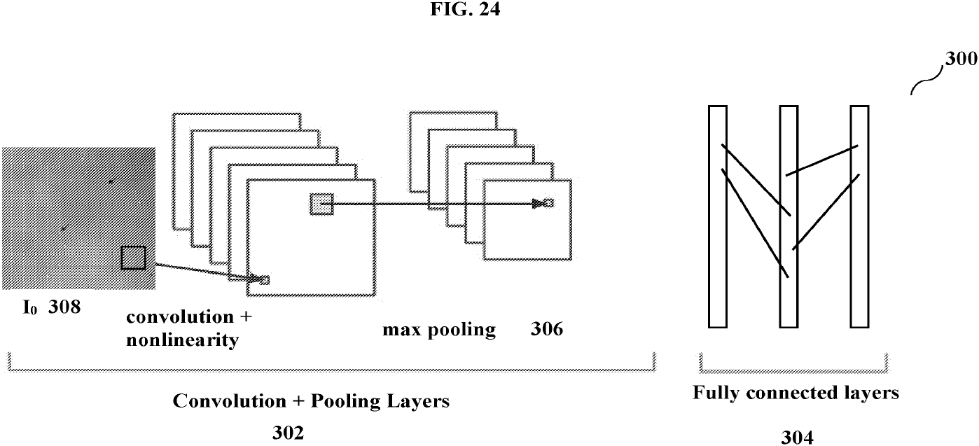

The input 202 may take any shape or form, as desired, for operation of the computer system 200, including the ability for selecting, entering, or otherwise specifying parameters consistent with operating the computer system 200. In some instances, the input 202 may be designed to receive data acquired with a testing system such as described above. Among the processing tasks for operating the computer system 200, at least one processor 204 may be configured to perform the method described above with respect to FIGS. 9 and 23.

The memory 206 may contain software 210, and may be configured for storage and retrieval of processed information and data to be processed by the processor 204. In some aspects, the software 210 may contain instructions directed to performing the method described above. In particular, the software may include, for example, instructions for acquiring or otherwise retrieving / receiving data. The software may also include instructions for implementing the neural network that is used to process such data that is provided. In certain configurations, the software may include instructions for training the neural network. In other configurations, the software may include instructions for retraining the neural network if desired. The software may thus provide the code for the data acquisition, transfer, processing, and storage operations that can be used to implement exemplary processes like the one represented in FIG. 21.

In yet another aspect, the compositions, systems, and methods provided herein are useful for a variety of clinical applications of microscopy-based AST (MAST). In one example, an automated system for microscopy-based antimicrobial susceptibility testing is used to screen patient blood cultures for the presence of bacteria and to diagnose conditions such as bacterial sepsis. Currently, AST is performed only after isolation of the presumptive bacterial colonies from positive culture broth, a process that itself takes at least one day. Since delay in appropriate therapy for bacterial sepsis increases patient mortality, applying the systems and methods of the present invention to rapidly screen blood cultures in less than 4 hours will provide for even more immediate and potentially life-saving results. For blood culture screening, positive blood culture broth is directly dispensed onto a culture substrate for MAST. Positive blood cultures typically contain >10⁶ organisms per ml, which is more than adequate for direct digital dispensing. In some cases, MAST according to systems and methods provided herein can be used in conjunction with rapid MALDI-TOF identification of organisms from positive blood cultures. Other rapid identification methods useful with the systems and methods provided herein include, without limitation, molecular detection through nucleic acid amplification methods; fluorescent in situ hybridization; other hybridization based detection methods; next generation sequencing; and rapid biochemical detection methods.

Another application of the systems and methods provided herein is direct microscopy-based antimicrobial susceptibility testing of urine collected from patients having or suspected of having complicated urinary tract infection (cUTI), which includes potentially life-threatening kidney infections associated with high levels of bacteria in urine (≥10⁵ organisms ml⁻ ¹). Many urinary tract pathogens (e.g., E. coli, Klebsiella) are now multi-drug resistant. Therefore, we envision use of direct MAST on urine samples to provide antimicrobial susceptibility testing results approximately 48 hours faster than traditional methods. In some cases, urine samples are subjected to spin column-based concentration techniques.

Another application of the systems and methods provided herein is direct microscopy-based antimicrobial susceptibility testing for Candida infections. Candida infections, especially bloodstream infections, are associated with high mortality and morbidity rates. In particular, delay in appropriate treatment has been associated with poor outcomes. According to standard protocols, Candida AST requires approximately 24-48 hours (e.g., 48 hours on the automated Vitek 2 (Biomeriuex) automated identification system, or 24-48 hours by manual broth microdilution methods). The systems and methods provided herein can be modified to determine antimicrobial susceptibility of this eukaryotic pathogen in less than 6 hours.

In another aspect, the compositions, systems, and methods provided herein can be used for direct Mycobacterium tuberculosis (TB) susceptibility testing. Using standard protocols, 2-4 weeks are required to isolate TB in liquid culture, and an additional 1-2 weeks or more are required for susceptibility testing once the organism grows. Accordingly, therefore it can take about 4 to about 8 weeks to obtain susceptibility results following sample collection using standard methodologies. The standard paradigm for TB therapy is the administration of at least two active agents in order for therapy to be effective. If only one active agent is used, TB will develop resistance to that agent during therapy and its future use in this patient and patients infected subsequently by this patient will be lost. With the rise in multidrug-resistant (MDR) TB and extensively-drug resistant (XDR) TB, there is a greater likelihood of a single or no drug activity in empiric regimens. As a result, in some places in the world, TB patients may be treated with up to 5 different medications until susceptibility results are known. Therefore, any mechanism to shorten the time for susceptibility results to be available would be extremely welcome in the world’s fight against TB.

Direct susceptibility testing of TB specimens has been advocated as a way to accelerate TB susceptibility testing. However, this requires great technical expertise to perform and, to date, is only available to specialty TB laboratories. The systems and methods provided herein can be modified to rapidly assess antimicrobial susceptibility of TB specimens. In some cases, the test samples are concentrated sputum specimens or other concentrated respiratory specimens. It may be appropriate in some cases to specifically treat such samples with N-acetyl cysteine and sodium hydroxide to kill off normal flora. The treated samples are neutralized and concentrated by centrifugation prior to digital dispensation onto micro-well surfaces for MAST analysis. A digital dispenser such as the HP D300 may be used to digitally dispense serial dilutions of all relevant TB antimicrobials onto the dispensed samples to allow assessment of both first and second line TB agents. In some cases, Mueller Hinton broth can be replaced by a typical TB base medium known in the field (e.g., Middlebrook broth) comprising nutritional supplements and antimicrobials to prevent growth of resident bacterial flora and fungi not killed by the sodium hydroxide treatment. Notably, microscopy-based detection of mycobacterial growth would significantly accelerate susceptibility determination. Preferably, microwell plates comprising test specimens are scanned at least once per day until susceptibility results are obtained. The combination of direct specimen testing and rapid microscopic assessment should greatly accelerate TB testing efforts and provide early critical information about drug regimens.

In another embodiment, the systems and methods provided herein can be used to accelerate susceptibility testing of isolated mycobacterial organisms. Mycobacterium tuberculosis and other mycobacteria may be isolated from primary specimens through culture in liquid broth (e.g., the BD MGIT™ Mycobacteria growth indicator system) or on solid medium. The positive culture broth or isolated mycobacterial colonies may then be applied using digital dispensing methodology along with any antimicrobials of interest and interrogated by microscopy on a daily basis to determine susceptibility results more rapidly than currently available by methods used in the field.

In another embodiment, the systems and methods provided herein can be used to automated and accelerate antiviral susceptibility testing. For example, viral particles may be digitally dispensed into microwells containing a susceptible host cell line. Viral particles could be serial diluted using digital dispensing technology to allow ready detection of plaque forming units or dispensed at a fixed quantity. Antivirals would then be added alone or in combination using digital dispensing technology. Viral cytopathic effect or other evidence for viral replication known in the field would then be detected via light or fluorescent microscopy or spectrophotometrically to assess therapeutic effects.

### Articles of Manufacture

In another aspect, the present invention provides articles of manufacture useful for automated microscopy-based antimicrobial susceptibility testing (MAST) according to the systems and methods provided herein. In some cases, the article of manufacture is or includes a preloaded cassette comprising one or more antimicrobial agents, including serial dilutions of one or more antimicrobial agents. In other cases, the article of manufacture is or includes a preloaded cassette comprising a cell culture medium. In exemplary embodiments, one or more pre-loaded cassettes are used in conjunction with a digital dispenser.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “consisting essentially of” shall have its ordinary meaning as used in the field of patent law. As used herein, “about” means within 5% of a stated concentration range or within 5% of a stated time frame.

It should also be understood that, unless clearly indicated to the contrary, in any methods claimed herein that include more than one step or act, the order of the steps or acts of the method is not necessarily limited to the order in which the steps or acts of the method are recited.

Having now described the invention, the same will be illustrated with reference to certain examples, which are included herein for illustration purposes only, and which are not intended to be limiting of the invention.

### EXAMPLES

Reference is now made to the following examples, which together with the above descriptions illustrate the invention in a non-limiting fashion.

### Example 1: Verification of an Automated, Digital Dispensing Platform for At-Will Broth Microdilution Microscopy-Based Antimicrobial Susceptibility Testing

We developed and verified a novel platform to enable hospital-based clinical laboratories to perform facile broth microdilution testing for any antimicrobial at will. Specifically, we make use of inkjet printing technology in the HP D300 digital dispensing system to automatically prepare two-fold serial dilutions of antimicrobials in 384-well microplate format followed by direct inoculation of bacteria. This high capacity format was combined with plate absorbance readings and automated data analysis to determine MICs. As proof-of-principle, we verified the performance characteristics of this method by testing representative clinical isolates of Enterobacteriaceae for susceptibility to ampicillin, cefazolin, ciprofloxacin, colistin, gentamicin, meropenem, and tetracycline in comparison to the CLSI broth microdilution gold standard (13).

Specifically, we evaluated the performance of the 384-well format, digital dispensing methodology in comparison to the broth microdilution reference method. Work was performed in two phases. In the first phase, a well-characterized set of control strains was used to test the reproducibility of the digital dispensing method compared to the broth microdilution reference. In the second, accuracy was evaluated using a large collection of clinical isolates. Our findings demonstrate that the D300 platform will enable hospital-based clinical microbiology laboratories to perform at-will testing of nearly any antimicrobial and thereby help address the antimicrobial testing gap.

Performance was verified by testing Enterobacteriaceae for susceptibility to ampicillin, cefazolin, ciprofloxacin, colistin, gentamicin, meropenem, and tetracycline in comparison to a broth microdilution reference standard. In precision studies, essential and categorical agreement were 96.8% and 98.3%, respectively. Furthermore, significantly fewer D300-based measurements were outside ± 1 dilution from the modal MIC, suggesting enhanced reproducibility. In accuracy studies performed using a large panel of curated clinical isolates, essential and categorical agreement; and very major, major, and minor errors were 94%, 96.6%, 0%, 0%, and 3.4%, respectively. Based on these promising initial results, it is anticipated that the D300-based methodology will enable hospital-based clinical microbiology laboratories to perform at-will broth microdilution testing of antimicrobials and address a critical testing gap.

### Materials and Methods

Bacterial strains and antimicrobials: Escherichia coli ATCC 25922, Enterobacter cloacae ATCC 13047, Klebsiella pneumoniae ATCC 13883, and Proteus mirabilis ATCC 702 were obtained from the American Type Culture Collection (Mannasas, VA). K. pneumonia BIDMC12A is a previously described, carbapenem resistant clinical isolate (14) expressing a KPC-3 carbapenemase. The eighty de-identified Enterobacteriaceae clinical isolates (Table 1) used for verification studies were collected at our institution under IRB-approved protocols. All strains were minimally passaged and stored at -80° C. prior to use in this study.

Ampicillin and tetracycline were from Thermo Fisher Scientific (Waltham, MA). Cefazolin was from Tokyo Chemical Industry (Portland, OR). Ciprofloxacin was from United States Biological (Salem, MA). Colistin sulfate was from Santa Cruz Biotechnology (Dallas, TX). Gentamicin was from Sigma-Aldrich. Meropenem was from ArkPharm (Libertyville, IL). Compounds used in manual broth microdilution testing were dissolved according to CLSI guidelines (3). Antibiotic stock solutions used for the digital dispensing method were dissolved in sterile water containing 0.3% polysorbate-20 (Sigma-Aldrich, St. 136 Louis, MO), as small amounts of surfactant are required for proper aqueous fluid handling by the D300 instrument. This surfactant becomes diluted to insignificant amounts during MIC testing. All antimicrobials were stored as aliquots at -20° C. and discarded after a single use.

Broth microdilution testing: Broth microdilution was performed using the colony suspension method according to CLSI guidelines (13). Colistin testing was consistent with the joint CLSI-EUCAST (European Committee on Antimicrobial Susceptibility Testing) Polymyxin Breakpoints Working Group guidelines (15). Serial two-fold dilutions of antimicrobials at double concentration were made in 96-well plates (Evergreen Scientific, Los Angeles, CA) using cation adjusted Mueller-Hinton broth (BD Diagnostics, Franklin Lakes, NJ) in a 50 µl volume. Inocula were prepared by suspending several bacterial colonies in sterile, cation-adjusted Mueller-Hinton broth and adjusting to a cell density of approximately 1 × 10⁶ CFU ml⁻¹ based on optical density at 600 nm (OD600). 50 µl of the adjusted suspension was added to the double concentration antimicrobial panels, bringing the bacteria to a final concentration of approximately 5 × 10⁵ CFU ml⁻¹ and antibiotics to final desired concentration. Panels were incubated at 37° C. in ambient air for 18-24 hours. MIC was defined as the lowest concentration of antimicrobial resulting in complete inhibition of growth as determined visually. Quality control of the reference method was verified on an ongoing basis during experiments by confirming that the MICs for E. coli ATCC 25922 tested during each experiment fell within quality assurance limits defined in CLSI guidelines (3).

Digital dispensing testing: Concentrated stocks of antibiotics were thawed and used directly to dispense two-fold dilutions into empty, flat bottomed, untreated polystyrene 384-well plates (Greiner Bio-One, Monroe, NC, part number 781186) using an HP D300 digital dispensing system 159 (Hewlett-Packard, Palo Alto, CA). Bacterial suspensions were prepared as for broth microdilution and adjusted directly to the final inoculum concentration of 5 × 10⁵ CFU ml⁻¹ in a 50 µl total assay volume. Incubation was for 18-24 hours at 37° C. in ambient air. Cell growth was quantified by measurement of optical density at 600 nm (OD600) with an Epoch microplate reader (BioTek, Winooski, VT). A custom python script was used to classify wells with OD600 ≧ 0.08 (approximately two-fold above typical background readings) as having bacterial growth. MICs were defined as the lowest concentration of antimicrobial resulting in OD600 < 0.08.

Precision analysis: A precision analysis was conducted according to established guidelines (16, 17). The reference broth microdilution method was repeated at least three times, and the digital dispensing method was repeated five times in triplicate for each antimicrobial/organism combination. All testing occurred on separate days with freshly prepared antimicrobial dilutions and independent inoculum preparations. Antimicrobial agents were used at the following concentration ranges: ampicillin from 0.06 to 128 µg ml⁻¹, cefazolin from 0.008 to 16 µg ml⁻¹, ciprofloxacin from 0.004 to 8 µg ml⁻¹, colistin from 0.06 to 32 µg ml⁻¹, gentamicin from 0.02 to 32 µg ml⁻¹, meropenem from 0.004 to 8 µg ml⁻¹, and tetracycline from 0.03 to 64 µg ml⁻¹. Antibiotic/organism combinations yielding growth in the highest concentration of antimicrobial tested or no growth at the lowest concentration of antimicrobial tested were considered off-scale. The modal MIC for both methods was determined using MIC values for on-scale measurements. Log2 difference of each measurement from the mode was recorded. The resulting distribution was plotted using Microsoft Excel 2010 (Microsoft, Redmond, WA).

To calculate precision categorical and essential agreement, the modal MIC from the reference method was recorded as the reference MIC. Each value determined by the digital dispensing method was compared with the reference MIC, and log2 differences were recorded. Off-scale measurements were not considered for evaluable essential agreement (“EA”) (17). Results from the digital dispensing method were considered to be in evaluable EA if they yielded an MIC ± 1 dilution from the reference MIC. Results were considered to be in overall EA if they were either (1) in evaluable EA, (2) both off-scale in the same direction, or (3) one measurement at the lowest or highest evaluable MIC tested and one measurement off-scale in the same direction. Results were considered in categorical agreement (CA) if both methods yielded the same susceptible/intermediate/resistant (S/I/R) interpretation. CLSI categorical interpretive criteria were used for ampicillin, cefazolin (parenteral), ciprofloxacin, gentamicin, meropenem, and tetracycline (3). EUCAST criteria were used for colistin (18).

Verification study: Digital dispensing and reference method testing were performed in parallel using the same inoculum preparation. Microdilution reference panels were pre-prepared and stored at -80° C. until use (less than 2 weeks). D300 test method panels were prepared fresh each day of use. Antimicrobial agents were used at the following concentration ranges: ampicillin from 0.13 to 256 µg ml⁻¹, cefazolin from 0.03 to 64 µg ml⁻¹, ciprofloxacin from 0.02 to 32 µg ml⁻¹, colistin from 0.13 to 64 µg ml⁻¹, gentamicin from 0.06 to 128 µg ml⁻¹, meropenem from 0.02 to 32 µg ml⁻¹, and tetracycline from 0.06 to 128 µg ml⁻¹.

Accuracy was evaluated using established guidelines (16, 17). The MIC determined by the digital dispensing method was compared with the MIC determined by the broth microdilution reference method. Essential agreement, overall essential agreement, and categorical agreement were evaluated as described in the precision study. Minor errors (MinE) were defined as either (1) a susceptible/resistant result from the test method and an intermediate result from the reference method or (2) an intermediate result from the test method and a susceptible/resistant result from the reference method. Major errors (ME) were defined as a resistant result from the test method and a susceptible result from 205 the reference method. Very major errors (VME) were defined as a susceptible result from the test method and a resistant result from the reference method.

Statistical analysis: Proportions of out-of-range (>1 two-fold dilution difference) to in range (≦ 1 two-fold dilution difference) measurements determined during precision analysis for the test and reference methods, respectively, were compared using the Fisher’s exact test with significance defined as a p < 0.05. 95% confidence intervals were calculated for essential and categorical agreement based on CLSI recommendations (19). All statistical analyses were performed in JMP 12.0.1 (SAS, Cary, NC).

### Results

We evaluated the precision and accuracy of 384-well format, broth microdilution panels prepared using automated, digital dispensing methodology compared to the manual, broth microdilution reference method.

Precision analysis: Published guidelines suggest testing precision using five separate strains including characterized control strains and representative multidrug resistant pathogens (16). Therefore, Enterobacter cloacae ATCC 13047, Escherichia coli ATCC 25922, Klebsiella pneumoniae ATCC 13883, and Proteus mirabilis ATCC 702 were selected for precision studies to provide a diversity of genera and antimicrobial susceptibility patterns. K. pneumoniae BIDMC12A was selected as a previously characterized, representative, carbapenem-resistant Enterobacteriaceae (14).

We chose to examine seven drugs, each potentially undermined by distinct antimicrobial resistance mechanisms and, with the exception of colistin, suggested by CLSI for primary or secondary reporting for Enterobacteriaceae (3). Specifically, ampicillin, cefazolin and gentamicin are suggested by CLSI as group “A” antimicrobials that should be primarily tested and reported. Ciprofloxacin and meropenem are CLSI “group B” antimicrobials recommended for primary testing and selective reporting. Tetracycline is a CLSI “group C” antimicrobial recommended for supplemental testing and as a predictor for doxycycline and minocycline susceptibility. Colistin is an agent of last resort that may be useful in treatment of carbapenem resistant Enterobacteriaceae.

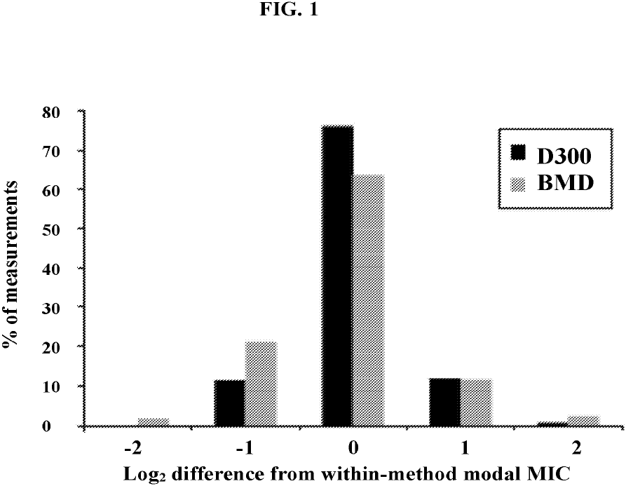

For precision analysis, the majority of measurements remained on-scale for the digital dispensing method (82.4 %) and reference method (85.1%), respectively. These measurements were used to create a distribution showing reproducibility of each method compared to the modal MIC (FIG. 1). Off-scale measurements, however, were not included as it is impossible to calculate Log2 differences from the modal MIC. Known colistin heteroresistance of K. pneumoniae ATCC 13883 (20) was detected in both broth microdilution and digital dispensing assays. This strain tested alternately as susceptible (MIC ≦ 0.25 µg ml⁻¹), resistant (MIC = 8 µg ml⁻¹), or un-interpretable based on multiple skipped wells. As such, colistin results from this organism were also not included in this or subsequent analysis.

Of 184 on-scale MIC measurements performed with the manual broth microdilution method, 96.2% fell within one doubling dilution of the modal MIC, 2.2% of measurements were two dilutions above, and 1.6% of measurements were 2 dilutions below the modal MIC, respectively. Average log2 difference from the modal MIC was -0.09 with a 95% confidence interval of -0.19 to 0.012. For 432 on-scale MIC tests performed with the digital dispensing method, 99.3% of results fell within ± 1 dilution of the modal MIC. 0.7% of measurements were two dilutions above and no measurements were two dilutions below the modal MIC. Average Log2 difference from the 251 modal MIC was 0.019 with a 95% confidence interval of -0.03 to 0.06. Comparison of the number of in-range to out of range measurements indicated the D300 method was significantly more precise than the manual broth microdilution reference method (Fisher’s exact test, p = 0.01).

Precision essential and categorical agreement was then determined (Table 2). Of all measurements, 97.8% were considered to be in evaluable EA. Evaluable EA for ampicillin, cefazolin, ciprofloxacin, gentamicin, meropenem, and tetracycline averaged 98.9%. EA for colistin was somewhat lower at 84.4%. However, all disagreements for colistin occurred where the reference MIC was ≧ 2 dilutions below the EUCAST-defined susceptibility breakpoint (2 µg mL⁻¹) (18).

14.3% of measurements were “off-scale” in the reference method and thus could not be utilized for evaluable essential agreement. These measurements were incorporated into an overall EA calculation. For ampicillin, cefazolin, ciprofloxacin, gentamicin, meropenem, and tetracycline overall EA averaged 99.1%. Colistin presented a lower overall EA of 88.3% with all disagreements again occurring at low levels of colistin (≧ 2 dilutions below the interpretive breakpoint for susceptibility).

For assessment of categorical agreement (CA), interpretive criteria from CLSI (ampicillin, parenteral cefazolin, ciprofloxacin, gentamicin, meropenem, and tetracycline) or EUCAST (colistin) were used to classify each reference MIC as susceptible, intermediate (where applicable) or resistant. The test and reference method were then compared. Categorical agreement was 100% for ampicillin, ciprofloxacin, colistin, gentamicin, meropenem, and tetracycline (Table 2). Frank colistin resistance of P. mirabilis ATCC 702 and E. cloacae ATCC 13047, and susceptibility of E. coli ATCC 25922 and K. pneumoniae BIDMC 12A were detected consistently.

Of note, cefazolin demonstrated a lower CA of 88% when assessed using current parenteral breakpoints (3). This contrasted with 97.8% evaluable EA. All categorical errors were minor and occurred in the two strains with reference MICs lying on a cefazolin breakpoint: the susceptibility breakpoint of 2 µg ml⁻¹ for E. coli ATCC 25922 and the intermediate breakpoint of 4 µg ml⁻¹ for P. mirabilis ATCC 702. Notably, the cefazolin quality control range itself for E. coli ATCC 25922 (1-4 µg ml⁻¹) (3) straddles the susceptible/intermediate demarcation. D300 values for all but one of these minor errors were in EA with the reference method. Therefore, relatively lower CA for cefazolin can be explained by selective examination in the precision study of strains straddling breakpoint cutoffs. If alternative oral cefazolin breakpoints for uncomplicated urinary tract infection were used (susceptible ≦ 16 µg ml⁻¹) (3), categorical agreement was 100% across all strains tested.

Verification: The verification study compared the accuracy of the D300 versus the reference method utilizing a curated collection of eighty minimally passaged, de-identified clinical strains from our institution. Based on reference microdilution testing, 93.8% of our strains showed non-susceptibility to ≥1 antimicrobial tested, and 43.8% were multidrug resistant based on the EUCAST definition of acquired resistance to ≧ 3 antimicrobial classes (21). A summary of the resistance spectrum for antimicrobials tested is shown in Table 1.

Antibiotic concentrations chosen for ampicillin, cefazolin, ciprofloxacin, gentamicin, meropenem, and tetracycline ranged from 3 dilutions above the CLSI-defined resistance breakpoint to 6 dilutions below the susceptibility breakpoint. These ranges exceeded those suggested by the FDA (17) to accommodate the goal of understanding how well the D300 and reference methods tracked at extreme ends of the dilution range. Colistin concentrations ranged from 4 dilutions above the EUCAST resistance breakpoint to 4 dilutions below the susceptibility breakpoint. Further dilutions of colistin were not made due to known binding of the molecule to plastic at low concentrations, resulting in unreliable MIC determinations (22, 23).

Using these extended ranges, the majority of measurements (68.8%) for all antimicrobials tested collectively by the reference method (n=555) were on-scale despite the high incidence of resistance among our strain set. The majority (80.4%) of the high off-scale results (n = 99) were for ampicillin, cefazolin, or ciprofloxacin. All low off-scale results (n = 50) were attributable to ciprofloxacin and meropenem, an expected result given the large splay between the susceptibility breakpoints for these drugs (1 µg ml⁻¹) and the typically very low MIC for susceptible strains (modal MICs = 0.03 µg ml⁻¹ for meropenem and ≦ 0.02 µg ml⁻¹ for ciprofloxacin).

In aggregate, 94.0% of evaluable digital dispensing method measurements were in EA (±1 dilution from the reference method). When off-scale measurements were included in the calculation, overall EA was 91.0%. Taken together, average evaluable EA for ampicillin, cefazolin, ciprofloxacin, gentamicin, meropenem, and tetracycline was 95.3% and overall EA was 93.8%. Colistin showed lower agreement with an evaluable EA of 90% and overall EA of 73.7%. Average CA was 96.6% and ranged from 92.3% to 100% for the antimicrobials tested as shown in Table 3. There were no major or very major errors identified. The minor error rate was 3.4% (n = 19). Notably, despite lower EA for colistin, CA for this antimicrobial was 100%.

### Discussion

Here we present verification data for a digital dispensing technology that enables generation of custom microdiluton antimicrobial susceptibility testing panels. Importantly, we found that this 384-well format method performed almost identically to reference MIC testing for seven different types of antimicrobials tested against several Enterobacteriaceae species. Specifically, precision EA (97.3%) and CA (98.2%) were well within the recommended >95% threshold suggested by CumiTech 31A (16) and FDA guidance documents (17). In addition, digital dispensing methodology demonstrated significantly lower variation from modal MIC during repeat measurements, suggesting enhanced reproducibility. For accuracy studies, EA, CA; and very major (VME), major (ME), and minor errors (MinE) were 94%, 96.6%, 0%, 0%, and 3.4%, respectively, within recommended target values of ≥90% for CA and EA; the combined threshold of ≤3% for ME and VME; and the combined threshold of ≤7% for minor and major errors (16). Therefore, the precision and accuracy of the D300 method was verified by generally accepted criteria against a reference gold standard.

We also examined performance for each antimicrobial individually to identify issues that might not be apparent in aggregate analysis. Not unexpectedly, issues with EA were identified for colistin in both precision and accuracy studies. Colistin is a lipopeptide antibiotic with a strong affinity for plastics used in antimicrobial susceptibility testing (23). The majority of colistin EA discrepancies (77.2%) occurred well below the susceptibility breakpoint, as observed in prior studies (22, 24). Our findings may relate, at least in part, to differential adsorbance of low levels of colistin in 384-well (test method) versus 96-well (reference method) plates. Prior evidence suggests that blocking microplate surfaces through addition of surfactants (e.g., polysorbate-80) to broth media may mitigate these effects (3, 13, 24). However, addition of surfactants is not currently recommended by CLSI/EUCAST (15) and was therefore not explored, especially as 342 CA was 100%. Of note, aqueous dispensing from the D300 requires inclusion of small amounts of polysorbate-20 in stock solutions. However, in our studies, polysorbate-20 became diluted to trivial amounts (< 0.002%) even at the highest antimicrobial concentrations tested and was neither expected nor observed to have any effect on results.

The D300 platform is based on inkjet printer technology that allows precise delivery of antimicrobials of interest to microplate wells in quantities ranging from 11 picoliters to 10 microliters per the manufacturer’s technical specifications (25). In this way, antimicrobial stock solutions can be used to set up a doubling-dilution series over a wide range of concentrations more than sufficient for any MIC determination scheme. Furthermore, the currently available T8+ compound cassettes can be loaded with up to 8 antimicrobials, each in a separate channel. Each of these channels can be used independently and at different times allowing flexibility. The instrument can dispense stock solutions dissolved in either aqueous solution or dimethyl sulfoxide (DMSO) per recommendations in CLSI guidelines (3).

To increase capacity for susceptibility testing, we verified functionality in 384-well plate format. However, the D300 instrument is equally capable, per specifications and based on our experience, of dispensing in either 96-well or 1536-well microplate formats, the former of which may be more practical in clinical antimicrobial susceptibility testing efforts. Notably, spectrophotometric absorbance readings followed by automated data analysis using custom scripts further eliminated operator-dependent interpretation. This is especially important for assigning results in the 384-microplate format, which would be very difficult to interpret visually. Lastly, we verified that the system can dispense into dry plates, which can be used immediately (as is this study) or frozen and used at a later time (data not shown). Therefore, it is possible to use digital dispensing technology to create custom MIC panels containing multiple antibiotics for either immediate or later use.

In terms of practical implementation in the clinical laboratory, it is useful to review (1) workflow relative to reference microdilution testing and (2) overall capacity for creation of MIC plates. CLSI M100-S26 provides a recommendation for preparation of a manual broth microdilution series (3). Briefly, it suggests creation of 4 dilutions from a stock solution followed by combination with three different volumes of media to create a thirteen-step dilution series. Practically, there are 24 micropipetting steps and 13 serological pipetting steps in this protocol. In total, a significant number of consumables are used in this procedure including at least 17 micropipette tips, 13 conical tubes, and a serological pipette.

This contrasts with a single micropipetting step to load a channel in a T8+ cassette. Of great importance for implementation in a hospital-based clinical laboratory setting is the time required to create a dilution series. Pipetting stock solution into a T8+ cassette channel, loading the cassette into the D300 instrument, recalling a protocol in the software user interface, and dispensing antimicrobials takes approximately two minutes. In contrast, the steps described above for setting up manual microdilution for a single antimicrobial were measured at 14.5 minutes. It would likely take significantly longer than that amount of time to perform in a traditional clinical microbiology laboratory and would become impractical for testing multiple isolates against several antimicrobials.

When comparing the 1 versus 37 pipetting steps required for creation of dilution series on the D300 versus the reference method, it is not surprising that the D300 was more precise (FIG. 1). In practical clinical usage, precision may be expected to be decreased given time constraints and variable operator experience. Therefore, the D300 methodology also showed advantage in terms of reproducibility. Based on the requirement for only a single pipetting step combined with automated data collection and interpretation, it should also be considered operator independent (16).

Both the manual and D300 method allow for antimicrobial dilution series to be stored for future use. Notably, a single T8+ cassette channel loaded fully with antimicrobial (10 µl) can dispense a large number of dilution series. Taking the example of meropenem and plating into a 384-well plate format, using a range of 3 dilutions below and 2 dilutions above the susceptible and resistance breakpoint, respectively, and an aqueous stock solution of 6.25 mg ml⁻¹ (used in this study), approximately 39 dilution series can be created. Alternative use of the high capacity D4+ cassette (250 µl) allows for creation of 144 dilution series from aqueous stocks. Therefore, the D300 can be used to set up a large number of pre-made panels that can be stored and used as needed.

In essence, the D300 methodology provides a highly automated way to set up a reference broth microdilution equivalent, and, therefore, we predict that it should perform adequately in most if not all situations where broth microdilution is used. We further predict that its use should extend to MIC testing of diverse types of organisms such as fungi and mycobacteria and include both traditional and direct susceptibility testing from primary specimens and blood cultures. Lastly, we have determined previously that the method facilitated combinatorial antimicrobial testing (synergy) with ease, even in far more complex experimental conditions than are used in traditional clinical microbiology laboratory-based testing (14, 26).

This study provides proof-of-concept for digital dispensing-based broth microdilution antimicrobial susceptibility testing. We expect this methodology will allow clinical laboratories to rapidly create custom panels of antimicrobials at will, including those not available in commercially available panels or formats. It will thereby enable hospital-based clinical laboratories to address the current, clinically unacceptable, antimicrobial testing gap.

TABLE 1

Summary of antimicrobial resistance found in strains used in the verification study

| Antibiotic | Non-susceptible strains n (%) |
|---|---|
| Ampicillin (AMP) | 65 (81.3) |
| Cefazolin (CFZ) | 51 (65.4) |
| Ciprofloxacin (CIP) | 35 (44.9) |
| Colistin (CL) | 17 (21.5) |
| Gentamicin (GEN) | 25 (31.3) |
| Meropenem (MEM) | 14 (17.5) |
| Tetracycline (TET) | 34 (42.5) |

*Categories based on CLSI criteria except for colistin which was based on EUCAST as no CLSI interpretive criteria exist for Enterobacteriaceae.

TABLE 2

Precision Analysis^{a}

| Antimicrobial | Number of measurements with Log₂ difference from reference MIC^{b} | | | | | Agreement % (CI^{c}) | | |
|---|---|---|---|---|---|---|---|---|
| | -1 | 0 | 1 | 2 | 3 | Overall Essential | Evaluable Essential | Categorical |
| AMP | 11 | 23 | 26 | 0 | 0 | 100 (95.1-100.0) | 100 (93.9-100) | 100 (95.1-100) |
| CFZ | 0 | 21 | 23 | 1 | 0 | 98.7 (92.8-99.8) | 97.8 (88.4-99.6) | 88 (78.7-93.6) |
| CIP | 0 | 22 | 37 | 1 | 0 | 98.7 (92.8-99.8) | 98.3 (91.1-99.7) | 100 (95.1-100) |
| CL | 0 | 22 | 16 | 5 | 2 | 88.3 (77.8-94.2) | 84.4 (71.2-92.2) | 100 (93.9-100) |
| GEN | 15 | 50 | 9 | 1 | 0 | 98.7 (92.8-99.8) | 98.7 (92.8-99.8) | 100 (95.1-100) |
| MEM | 0 | 25 | 34 | 1 | 0 | 98.7 (92.8-99.8) | 98.3 (91.1-99.7) | 100 (95.1-100) |
| TET | 34 | 39 | 2 | 0 | 0 | 100 (95.1-100) | 100 (95.1-100) | 100 (95.1-100) |
| Total n (%, CI) | 60 (14.3) | 202 (48.1) | 147 (35) | 9 (2.1) | 2 (0.5) | 499 (97.8, 96.2-98.8) | 409 (97.3, 95.3-98.5) | 501 (98.2, 96.7-99.1) |

^{a}Precision analysis performed on E. coli ATCC 25922, K. pneumoniae ATCC 13883, E. cloacae ATCC 13047, P. mirabilis ATCC 702 and K. pneumoniae BIDMC12A.
^{b}Only evaluable comparisons included for which both DDM and BMD MIC measurements were within dilution ranges tested.
^{c}95% Confidence interval

TABLE 3

Results of Verification Study

| Antimicrobial | Number of strains with Log₂ difference from reference MIC^{a} | | | | | Agreement % (CI^{b}) | | |
|---|---|---|---|---|---|---|---|---|
| | —2 | —1 | 0 | 1 | 2 | Overall Essential | Evaluable Essential | Categorical |
| AMP | 0 | 2 | 10 | 19 | 3 | 95.0 (87.8-98.0) | 91.2 (77.0-97.0) | 95 (87.8-98.0) |
| CFZ | 0 | 9 | 34 | 5 | 0 | 100 (95.3-100.0) | 100 (92.6-100) | 92.3 (84.2-96.4) |
| CIP | 0 | 2 | 12 | 10 | 0 | 94.9 (87.5-97.9) | 100 (86.2-100) | 98.7 (93.1-99.8) |
| CL | 1 | 3 | 17 | 7 | 2 | 73.7 (63.2-82.1) | 90 (73.6-96.4) | 100 (95.4-100) |
| GEN | 1 | 12 | 36 | 22 | 6 | 90.0 (81.5-94.8) | 91 (82.4-95.5) | 97.5 (91.3-99.3) |
| MEM | 0 | 4 | 32 | 17 | 1 | 96.3 (89.5-98.7) | 98.1 (90.2-99.7) | 97.5 (91.3-99.3) |
| TET | 1 | 5 | 41 | 17 | 5 | 86.3 (77.0-92.1) | 91.3 (82.3-96.0) | 95 (87.8-98.0) |
| Total n (%, CI) | 3 (0.9) | 37 (11.0) | 182 (54.2) | 97 (28.9) | 17 (5.0) | 505 (91.0, 88.1-93.0) | 315 (94.0, 91.0-96.1) | 536 (96.6, 94.7-97.8) |

^{a}Only evaluable comparisons included for which both DDM and BMD measurements were within the dilution ranges tested.
^{b}95% Confidence interval

### Example 2: Digital Dispensing Technology

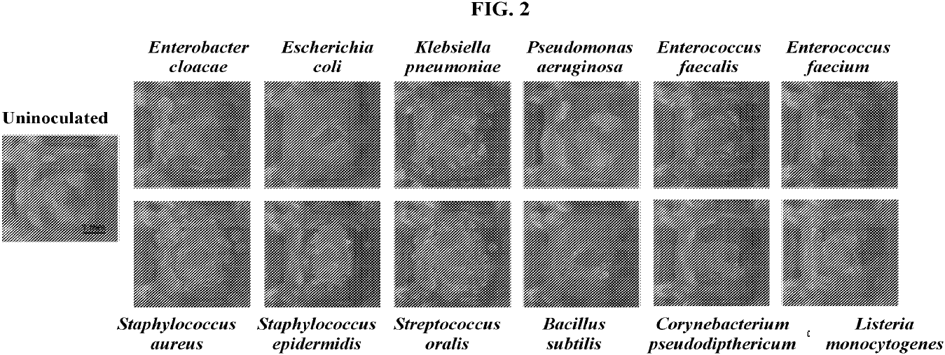

This example describes development of digital dispensing technology for automated inkjet dispensing of bacterial cells. This step is critical to generation of MIC assays in 384-well plates as manual inoculation of each well would be technically challenging, especially when assaying multiple antibiotic/organism combinations. Furthermore, the precise placement of organisms afforded by inkjet application in defined locations in each well also speeds later imaging steps. In preliminary experiments, we evaluated suitability of solidified Mueller-Hinton medium containing 20% poloxamer 407 as a growth surface for automatically dispensed bacterial cells of 10 genera representing almost all clinically relevant bacterial groups. As shown in FIG. 2, we observed robust growth of all tested bacteria, including Gram-negative rods (Enterobacter, Escherichia, Klebsiella, Pseudomonas), Gram-positive rods (Bacillus, Corynebacterium, Listeria), and Gram-positive cocci in clusters (Staphylococcus) or chains (Streptococcus, Enterococcus).

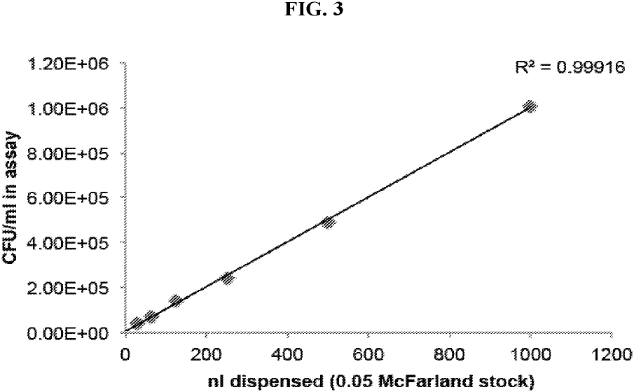

It was determined that dispensing of bacteria is very accurate and has a very high R-squared across linear range of dispensing. See FIG. 3. Our data demonstrate that the D300 can dispense microorganisms accurately at different dispense volumes. Furthermore, D300 inkjet technology and dispensing is compatible with viability of the organisms. Colony counts obtained were completely as expected based on the density of the stock solution of the E. coli quality control strain shown.

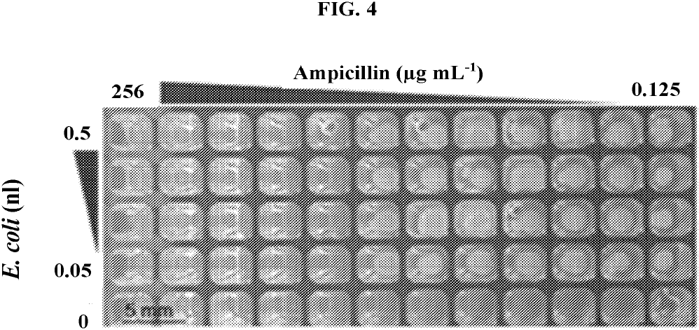

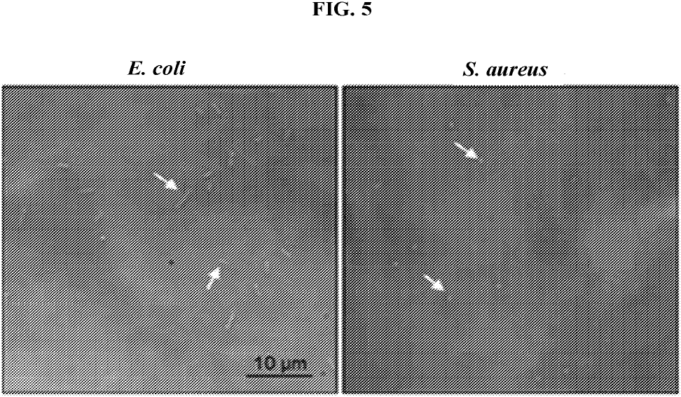

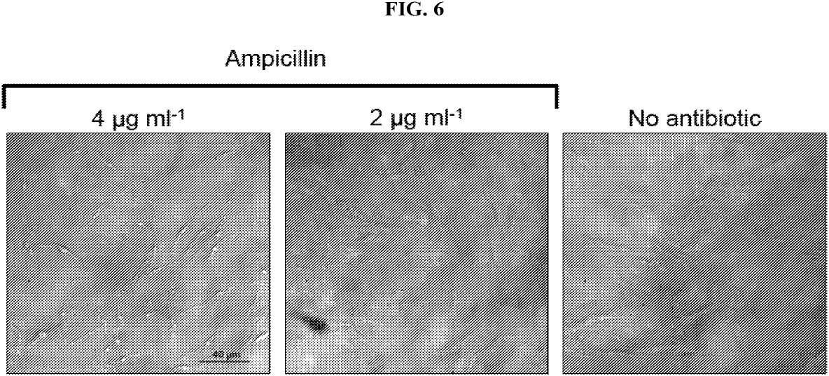

As proof of concept, we prepared plates containing a dilutions series of antimicrobials and D300 inoculated bacterial cells, followed by overnight incubation. We observed robust colony growth inside the wells and were able to read a reproducible and accurate MIC using bacterial inocula ranging from 30 nl to 0.05 nl volumes (see FIG. 4).

We performed preliminary automated imaging experiments on the Cell Observer microscope (Zeiss, Oberkochen, Germany) (37° C., 640x magnification, DIC). For these experiments, we utilized E. coli ATCC 25922 and S. aureus ATCC 25923 (FIG. 5), prototypical Gram-negative and Gram-positive pathogens. Notably, these strains are standard quality control strains used in clinical laboratories for validation of antimicrobial susceptibility testing systems. Imaging of an entire well indicated that, after D300 dispensing, bacteria are restricted to an approximately 2 mm² spot in the center of each well, confirming the accuracy of our digital dispensing method. Therefore, we conducted automated imaging of the center of 20 wells using 20-50 z-slices of approximately 1 µm thickness. Each image represented approximately 43,500 µm². After collapsing z-slices into a single image per well, cells were readily detectable and distributed evenly throughout the field at a density of 10-30 cells per 1000 µm² in all wells evaluated.

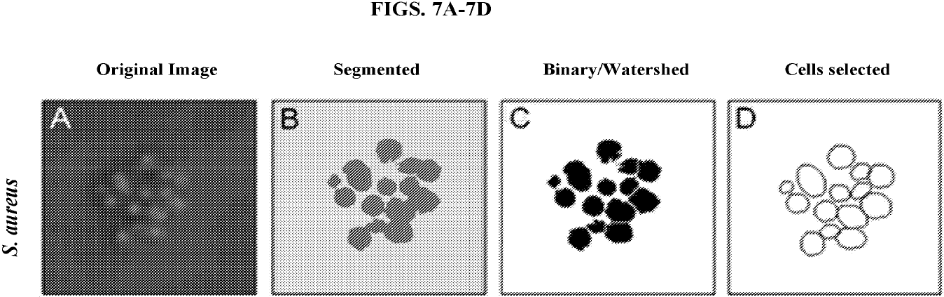

As proof of concept and preliminary data for image analysis-based direct cell counting, we utilized a machine learning algorithm (Trainable Weka as contained within the Fiji implementation of ImageJ) (Schindelin et al., Nature Methods 9:676-682 (2012)). A training set of 200 manually segmented S. aureus cells was used to establish a model for cell detection. This model accepts raw image data as input (FIG. 7A), classifies each pixel of an image as containing “cells” or “background” resulting in a segmented image (FIG. 7B). The segmented image is then converted to a black and white binary format. A watershed algorithm refines the segmentation by defining borders where two or more cells are in contact with one another and prevents later erroneous single counting of these otherwise fused objects (FIG. 7C). The particle analysis function of ImageJ is then used to identify particles >0.25 µm² as individual bacteria (FIG. 7D). Identified bacteria are subsequently counted and their aggregate surface area calculated.

Our model for automated segmentation was tested using a challenge set of 610 manually counted S. aureus cells across three images which had not been used as a component of the training set. The automated method demonstrated 91% sensitivity and 97% specificity. Of note, specificity was robust despite variable background.

Using our cell counting model, we compared images of S. aureus grown on inhibitory and sub-inhibitory concentrations of antibiotics. At 0.5 × the MIC (ampicillin), we detected 242 cells occupying 555 µm². At the MIC, cell numbers were reduced 5-fold to 51 cells occupying 47 µm², indicating that antimicrobial growth inhibition of S. aureus is readily detectable at time points as early as 4 hours. At the same time point we observed that E. coli cells in wells with sub-inhibitory concentrations of antimicrobial formed large microcolonies, making analysis of individual cells difficult. In cases where individual cells could not be segmented, a machine learning algorithm similar to that outlined in (d) reliably quantifies relative cell coverage in each field.

References for Examples 1 and 2:
1. Tzouvelekis LS, Markogiannakis A, Piperaki E, Souli M, Daikos GL. 2014. Treating infections caused by carbapenemase-producing Enterobacteriaceae. Clinical microbiology and infection : the official publication of the European Society of Clinical Microbiology and Infectious Diseases 20:862-872.
2. Tan TY, Ng LS. 2006. Comparison of three standardized disc susceptibility testing methods for colistin. The Journal of antimicrobial chemotherapy 58:864-867.
3. Clinical and Laboratory Standards Institute. 2016. Performance standards for antimicrobial susceptibility testing; twenty-fifth informational supplement. CLSI document M100-S26. Clinical and Laboratory Standards Institute, Wayne, PA.
4. Dafopoulou K, Zarkotou O, Dimitroulia E, Hadjichristodoulou C, Gennimata V, Pournaras S, Tsakris A. 2015. Comparative Evaluation of Colistin Susceptibility Testing Methods among Carbapenem-Nonsusceptible Klebsiella pneumoniae and Acinetobacter baumannii Clinical Isolates. Antimicrobial agents and chemotherapy 59:4625-4630.
5. Center for Disease Dynamics Economics and Policy. 2015. State of the World’s Antibiotics, 2015. CDDEP, Washington, DC.
6. Weiner LM, Fridkin SK, Aponte-Torres Z, Avery L, Coffin N, Dudeck MA, Edwards JR, Jernigan JA, Konnor R, Soe MM, Peterson K, McDonald LC. 2016. Vital Signs: Preventing Antibiotic-Resistant Infections in Hospitals - United States, 2014. MMWR. Morbidity and mortality weekly report 65:235-241.
7. Bradford PA, Kazmierczak KM, Biedenbach DJ, Wise MG, Hackel M, Sahm DF. 2015. Correlation of beta-Lactamase Production and Colistin Resistance among Enterobacteriaceae Isolates from a Global Surveillance Program. Antimicrobial agents and chemotherapy 60:1385-1392.
8. Food and Drug Administration. 2014. Guidance for Industry: Expedited Programs for Serious Conditions - Drugs and Biologics. Food and Drug Administration, Rockville, MD.
9. Zhanel GG, Lawson CD, Adam H, Schweizer F, Zelenitsky S, Lagace-Wiens PR, Denisuik A, Rubinstein E, Gin AS, Hoban DJ, Lynch JP, 3rd, Karlowsky JA. 2013. Ceftazidime-avibactam: a novel cephalosporin/beta-lactamase inhibitor combination. Drugs 73:159-177.
10. Zhanel GG, Chung P, Adam H, Zelenitsky S, Denisuik A, Schweizer F, Lagace-Wiens PR, Rubinstein E, Gin AS, Walkty A, Hoban DJ, Lynch JP, 3rd, Karlowsky JA. 2014. Ceftolozane/tazobactam: a novel cephalosporin/beta-lactamase inhibitor combination with activity against multidrug-resistant gram-negative bacilli. Drugs 74:31-51.
11. Food and Drug Administration, posting date. Prescribing Information for ZERBAXA (ceftolozane/tazobactam) for Injection. [Online.]
12. Food and Drug Administration, posting date. Prescribing Information for AVYCAZ (ceftazidime-avibactam) for injection. [Online.]
13. Clinical and Laboratory Standards Institute. 2015. Methods for dilution antimicrobial susceptibility tests for bacteria that grow aerobically; approved standard - tenth edition. CLSI document M07-A10. Clinical and Laboratory Standards Institute, Wayne, PA.
14. Smith KP, Kirby JE. 2016. Validation of a High-Throughput Screening Assay for Identification of Adjunctive and Directly Acting Antimicrobials Targeting Carbapenem-Resistant Enterobacteriaceae. Assay and drug development technologies 4:194-206.
15. Chiaraviglio L, Kirby JE. 2015. High-Throughput Intracellular Antimicrobial Susceptibility Testing of Legionella pneumophila. Antimicrobial agents and chemotherapy 59:7517-7529.
16. Tecan Inc. 2016, posting date. Tecan D300e Digital Dispenser - Specification. [Online.]
17. Schindelin J, Arganda-Carreras I, Frise E, Kaynig V, Longair M, Pietzsch T, Preibisch S, Rueden C, Saalfeld S, Schmid B, Tinevez JY, White DJ, Hartenstein V, Eliceiri K, Tomancak P, Cardona A. 2012. Fiji: an open-source platform for biological-image analysis. Nature methods 9:676-682.

### Example 3 - Linearity and Precision of Digital Dispensing of Pathogens

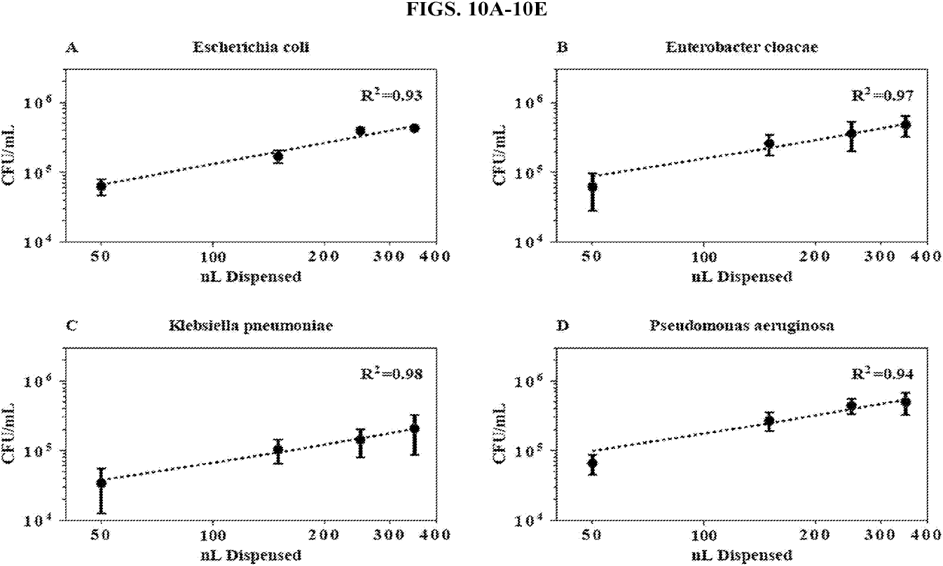

This section demonstrates linearity of digital dispensing for five major Gram-negative pathogens of significant medical concern: Escherichia coli,Enterobacter cloacae, Klebsiella pneumoniae, Pseudomonas aeruginosa, and Acinetobacter baumannii. Organisms were grown overnight at 37° C. in ambient air on tryptic soy agar containing 5% sheep’s blood and suspended to 0.5 McFarland in sterile 0.9% NaCl containing 0.3% polysorbate-20 using a handheld colorimeter. This suspension was added directly to T8+ or D4+ cassettes. Varying amounts of the suspension were dispensed into one quadrant (96-wells) of a 384-well plate with each well containing 50 µl of sterile Mueller-Hinton broth. Immediately after dispensing, we selected three inoculated wells (the first well dispensed, the 48th well dispensed, and the 96th well dispensed) for plate count to quantify the total number of viable bacteria in the well. The experiment was performed on three separate days. A standard curve was generated for each organism relating volume dispensed to colony forming units (CFU) per mL recovered from the inoculated well (FIGS. 10A-10E). No significant differences were observed between the three wells analyzed (ANOVA, p>0.05) and R² values were >0.9 for all organisms tested.

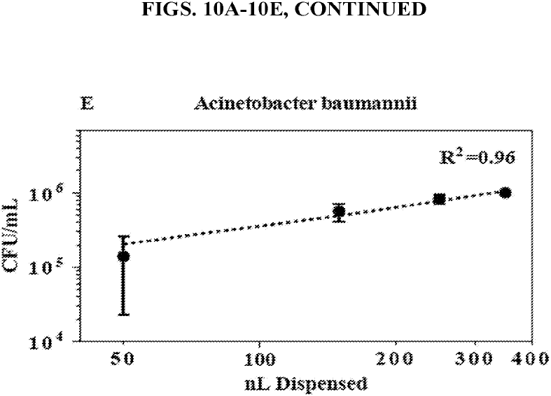

The MAST assay requires that bacteria can be dispensed into specific locations within a well of a 384-well plate and that those bacteria can then be reliably imaged following dispensing. Initial experiments demonstrated notable spatial precision. Specifically, bacteria could be spotted in the center of the well. Organisms were not noted outside this central area. FIGS. 11A-11B show Staphylococcus aureus, a representative Gram-positive pathogen, spotted in the center of a well from a 384-well plate on top of a solidified poloxamer growth surface. After four hours of incubation, microcolonies (grape-like clusters of cocci) were observed in the center of the well (in the geographic target zone), but not on the periphery of the well (outside of the geographic target zone), demonstrating predictable geographic placement of the bacteria.

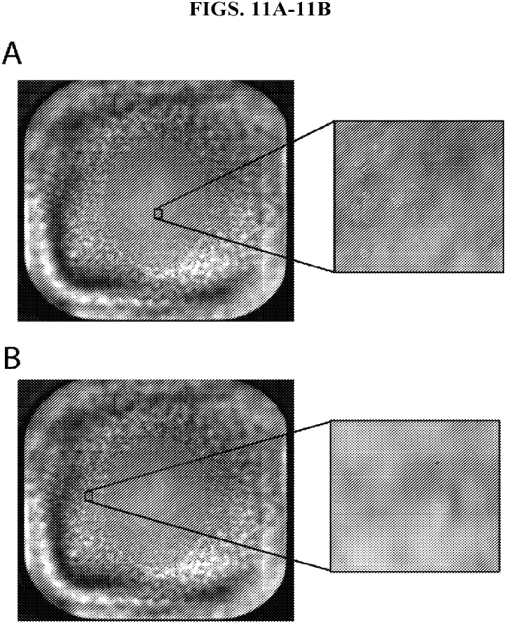

We evaluated the spatial precision of bacterial dispensing using 5 clinically relevant Gram-negative bacteria: Escherichia coli, Enterobacter cloacae, Klebsiella pneumoniae, Pseudomonas aeruginosa, andAcinetobacter baumannii. Each organism was grown overnight at 37° C. in ambient air and standardized to a density equal to 0.5 McFarland in 0.9% NaCl and 0.3% polysorbate-20. The suspension was diluted 1:5, and 200 nL was dispensed using the HP D300 into the center of a microwell containing cation-adjusted Mueller Hinton broth solidified with poloxamer 407. After dispensing (and without incubation) the central field of each microwell was imaged with a Zeiss Cell Observer microscope in bright field mode using a 40X long working distance objective. Cells were counted manually in each of 12 wells spanning the central 240 wells of a 384-well plate (the outer two rows were omitted due to limitations in automated stage movement). The experiment was repeated in triplicate on separate days. Average number of cells per field was consistent between experiments (FIG. 12).

The following section demonstrates successful automated image collection using automated microscopy of bacteria growing on solid microwell surfaces. Briefly, antibiotics are added to solidified microwell surfaces which are subsequently inoculated with 200 nL of a standardized bacterial suspension. Both antibiotic and bacterial cell dispensing are performed using the HP D300 system. Following incubation at 35±2° C., plates are imaged on a Zeiss Cell Observer microscope operating in brightfield mode with a 40X long working distance objective. The microscope is equipped with an automated stage. The x and y coordinates of each well of the 384-well plate are pre-loaded into the microscope control program (Zeiss Zen Blue) and do not need to be redefined for each experiment. The z-position of the poloxamer surface in the first well is set manually after which the software directs the stage to each subsequent well, collecting a z-series (20 slices at 2 µM per slice with the center of the z-series defined by the microscope’s autofocus feature) with no further operator intervention. Z-series are collapsed using the Extended Depth of Focus feature in the Zeiss Zen Blue software and saved as individual images. Images of bacterial cells grown at the minimal inhibitory concentration demonstrate characteristic patterns of growth inhibition (FIG. 13).

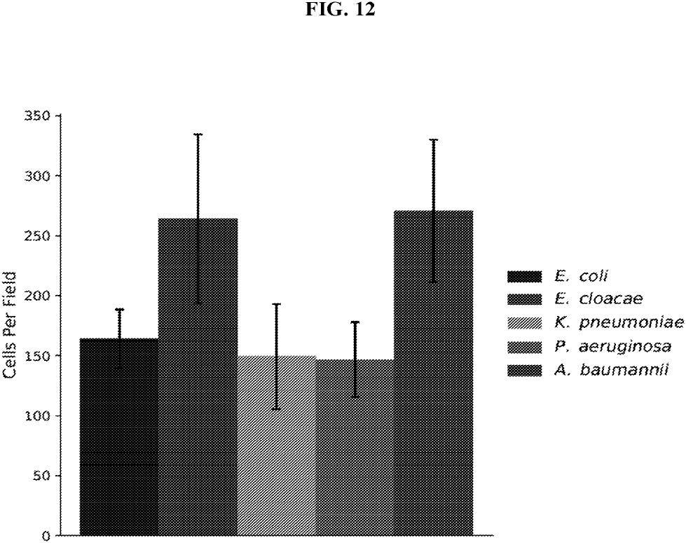

Each image was annotated through use of a custom Python script that correlates modified D300 logfiles and Zeiss Zen Blue image export data. This script changes the filename of each image to contain relevant information including organism, antibiotic concentration, and date. Following acquisition and annotation, images need to be evaluated and classified as “growth” or “no growth”. To automate image classification, we used 3,202 images collected with our automated protocol to train a deep convolutional neural network based on the VGG architecture (Simonyan & Zisserman, Very deep convolutional networks for large-scale image recognition. ICLR 2015 2015, available at arxiv.org/abs/1409.1556 on the World Wide Web). The neural network showed ~90% classification accuracy on a per-image-crop basis, thus providing evidence of feasibility for automated classification of MAST images (FIG. 14). Therefore, the technology as a whole is able to detect inhibition of bacterial growth at early time points with sufficient quality for machine learning classification.

### Example 4: Detecting Inoculum Effect Using Digital Dispensing Technology

Bacterial antimicrobial susceptibility testing is typically performed under defined conditions delineated by organizations such as the Clinical and Laboratories Standards Institute (CLSI) and the US Food and Drug Administration. For these standardized assays, organisms are suspended at a particular concentration, specifically 5 × 10⁵ colony forming units per ml. Doubling dilutions of antibiotics are mixed with organisms and liquid growth medium. The minimal concentration of antimicrobial that causes visible growth inhibition after 16-20 hours of incubation is deemed the minimal inhibitory concentration or the MIC. The MIC is predictive of patient response to therapy. Tables produced by organizations such as CLSI are used to interpret the MIC and assign categories of susceptible (S), intermediate (I), or resistant (R), based on the likelihood of therapeutic success. The relationship of therapeutic success to the MIC underlying CLSI interpretive tables is determined through pharmacodynamic studies, pharmacokinetic modeling, and past cumulative clinical experience with the particular antimicrobial agent and organism.

However, there are classes of antimicrobial-organism combinations that respond in a highly variable way when different concentrations of suspended organisms are used in testing. Specifically, the MICs will increase dramatically when somewhat higher concentrations of organisms are tested, for example, at 5 × 10⁶ colony forming units per ml (in this case 10× the standard inoculum). This phenomenon is called the inoculum effect. It has been noted that organisms in some types of human and veterinary infection (e.g., abscesses) may reach much higher numbers/densities/concentrations than standardly tested during in vitro susceptibility testing. Therefore, the inoculum effect may have direct clinical implication (reviewed by Brook et al) (Brook, Reviews of infectious diseases 1989, 11(3):361-368).

The inoculum effect is especially prominent for β-lactam antibiotics when bacterial strains contain certain β-lactamases. Importantly, the presence of an inoculum effect may predict therapeutic failure, a notion supported by in vivo animal model testing (Soriano et al., Europ. J. Clin. Microbiol. Infectious Diseases 1988, 7(3):410-412; Docobo-Perez et al., Antimicrobial Agents and Chemotherapy 2013, 57(5):2109-2113). For example, for Klebsiella pneumoniae strains expressing extended spectrum β-lactamases, the presence of an in vitro inoculum effect predicted decreased in vivo survival.⁵ Specifically, during in vitro antimicrobial susceptibility testing, strains demonstrated a significant inoculum effect for piperacillin/tazobactam and an insignificant inoculum effect for meropenem. Correspondingly, mice infected with a low bacterial inoculum survived after treatment with either piperacillin/tazobactram or meropenem, in contrast to untreated controls which all died (Harada et al., Europ. J. Clin. Microbiol. Infectious Diseases 2014, 20(11):0831-839). In contrast, all mice infected with a high dose inoculum died after treatment with piperacillin/tazobactram. However, all mice infected with a high dose inoculum survived after treatment with meropenem. Therefore, the presence of an in vitro inoculum effect predicted therapeutic failure during high inoculum infection.

There is currently no practical way to test for an inoculum effect within the workflow of a clinical laboratory, as such testing would require a highly laborious manual process of testing different inoculum levels with several potential antimicrobials. In this section, we demonstrate using digital dispensing technology to test desired concentrations of bacterial inoculum at will. By varying the droplet size during digital printing of a bacterial suspension as described in the preceding section, a desired inoculum of bacteria can be added to each testing well. More specifically, doubling dilutions of bacterial inocula can be performed if desired, thereby establishing the relationship between inoculum and MIC through an inoculum dose-response curve.

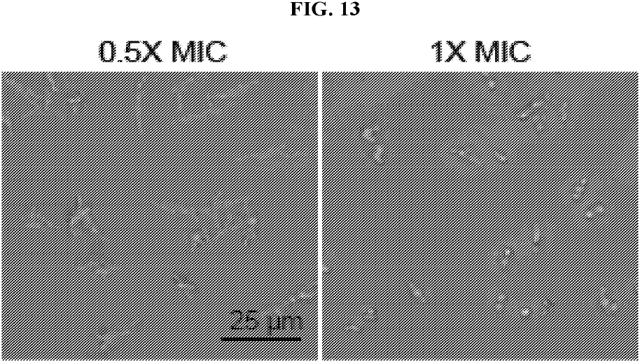

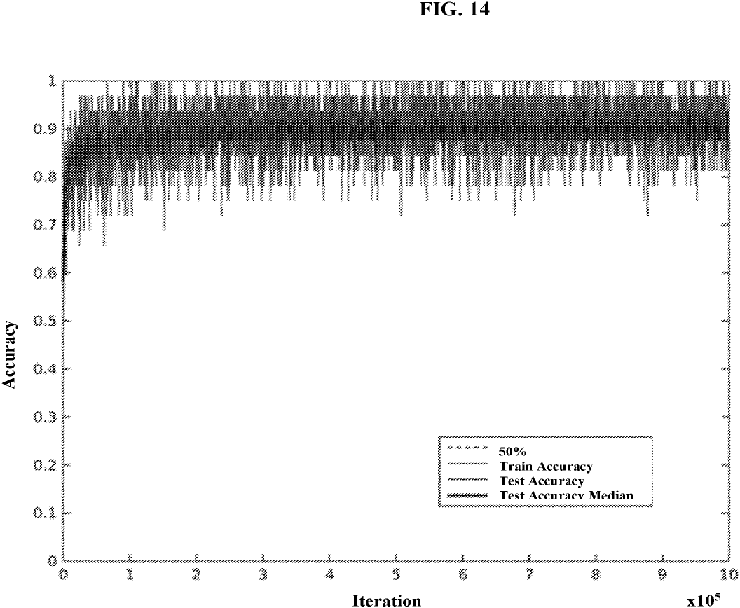

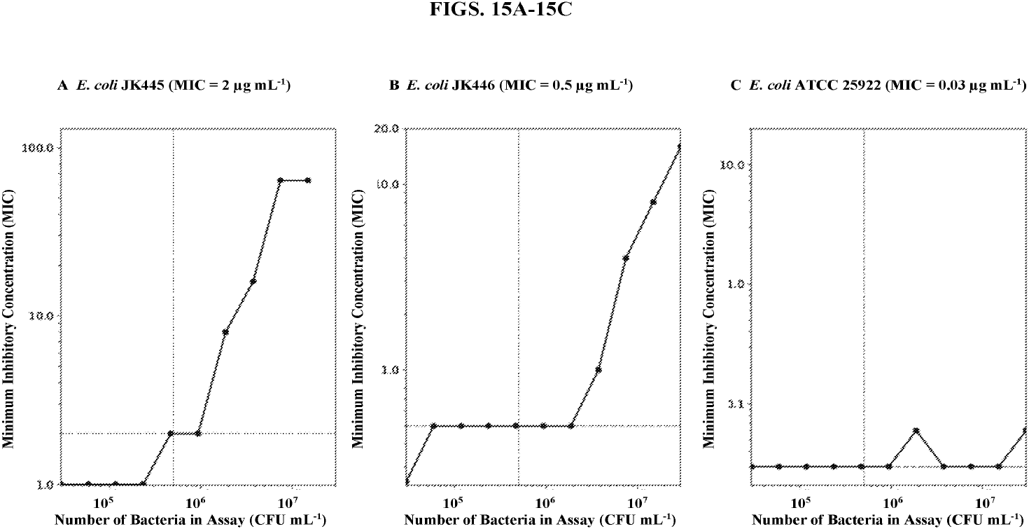

We investigated the inoculum effect for cefepime (a fourth generation cephalosporin) on 14 Enterobacteriaceae strains presumptively expressing extended spectrum beta-lactamase enzymes based on third generation cephalosporin resistance. Bacteria and antibiotics were both dispensed as orthogonal, combinatorial two-fold dilution series using our digital dispensing method to create a checkerboard array. We found that the inoculum effect as measured by the inkjet printing method was pronounced and manifest by highly elevated MICs when using a higher than CLSI-recommended inoculum and manifest by somewhat lower MICs when using a lower than CLSI-recommended inoculum (FIGS. 15A-15C). We envision in a future clinical test in which only selected inoculum concentrations for each organism-antimicrobial combinations would be tested. Specifically, testing would only be performed in instances where inoculum effect is known to vary (presence versus absence, e.g., FIGS. 15A and 15B versus FIG. 15C) and at the most discriminatory inoculum concentrations. In this way only a few extra wells would need to be tested in a susceptibility panel to elucidate potential inoculum effects of concern.

With the proliferation of resistance elements associated with emerging antimicrobial resistance, inoculum effects will become increasing more common in clinical strains. Using inkjet technology as outlined, a simple test could thereby set up to identify therapies that will be subject to an inoculum effect that otherwise would not be appreciated through standard MIC testing. We believe that this information will be extremely valuable in guiding clinicians towards or away from therapies based on presence or absence of inoculum effect. Clinicians would be able to take into consideration estimated organism burden during different types of infection and choose not to use antimicrobials (that previously would have been classified as “susceptible” using traditional antimicrobial susceptibility testing) that would predictably fail in the presence of high bacterial numbers in tissues.

### Example 5: Use of Digital Dispensing Technology to Investigate Checkerboard Antimicrobial Synergy

Synergistic combination antimicrobial therapy may provide new options for treatment of multidrug-resistant infections. However, facile methods to perform synergy testing in a clinically actionable time frame are unavailable. This example demonstrates use of digital dispensing technology for comprehensive combinatorial checkerboard testing of antimicrobials against carbapenem-resistantEnterobacteriaceae (CRE). As described in the following paragraphs, digital dispensing technology provides for automated addition of the exact amount of antimicrobial required in each well of a doubling dilution array directly from an antimicrobial stock solution, greatly simplifying assay setup. This work establishes the foundation for future systematic, broad-range investigations into antibiotic synergy for CRE, emphasizes the need for individualized synergy testing, and demonstrates the utility of digital printer-based technology for the performance of automated antimicrobial synergy assays.

### Materials and Methods

Bacterial strains: The 10 de-identified CRE clinical isolates used in the study were collected at our institution under Institutional Review Board-approved protocols and were sequenced through the carbapenem-resistant Enterobacteriaceae genome initiative at the Broad Institute (Cambridge, MA). All contained a Klebsiella pneumoniae carbapenemase (bla_{KPC}) gene and were colony-purified, minimally passaged, and stored at -80° C. prior to use in this study. Escherichia coli ATCC 25922 was obtained from the ATCC (Manassas, VA).

Antimicrobial agents: Antimicrobials were obtained from the following suppliers: Sigma-Aldrich, St. Louis, MO (levofloxacin, chloramphenicol, fosfomycin, gentamicin); Alfa Aesar, Tewksbury, MA (gentamicin); Ark Pharm, Libertyville, IL (meropenem); MP Biomedicals, Santa Ana, CA (aztreonam); Research Products International, Mount Prospect, IL (trimethoprim and ertapenem); Chem Impex International, Wood Dale, IL (sulfamethoxazole, minocycline, cefepime); Santa Cruz Biotechnology, Santa Cruz, CA and Alfa Aesar (colistin); and Fisher Scientific, Pittsburgh, PA (rifampin). Antibiotic stock solutions used in reference broth microdilution testing were dissolved according to CLSI guidelines,¹² with the exception of trimethoprim and sulfamethoxazole, which were dissolved in DMSO (Sigma-Aldrich, St. Louis, MO) as they were not soluble in CLSI-recommended solvents at the concentrations required. Antibiotic stock solutions used for the HP D300 digital dispensing method were dissolved in either sterile water according to CLSI guidelines¹² with the addition of 0.3% polysorbate 20 (P-20; Sigma-Aldrich, St. Louis, MO) or in DMSO (chloramphenicol, trimethoprim, sulfamethoxazole, and rifampin). The DMSO concentration ranged from 0.0008% to 0.968%, below the CLSI-recommended maximum concentration of 1%.¹² P-20 is required for proper fluid handling of aqueous antimicrobial stock solutions by the D300 instrument as part of the digital dispensing method (DDM) used for setting up checkerboard arrays. The final concentrations of surfactant in microdilution wells ranged from 3.1 x 10⁻⁷% to 0.015%. Of note, a different surfactant, polysorbate 80 (P-80) at a concentration of 0.002% has been noted to lower colistin MICs for organisms with colistin MICs of <2 in standard broth microdilution (BMD) assays.^{13,} ¹⁴ However, in our assays, P-20 was only introduced in assay wells at concentrations ≥0.002% when colistin concentrations were ≥64 µg/mL (with the exception of colistin at ≥2 µg/mL in combination with aztreonam and levofloxacin at ≥256 µg/mL and ≥128 µg/mL, respectively), and therefore was considered unlikely to interfere with assays. Our laboratory previously demonstrated that P-20 at all concentrations tested (up to 0.0015%) had no effect on DDM results in comparison to reference BMD.¹¹ Therefore P-20 should have no discernible effect on MIC values, as supported by the high essential agreement between DDM and BMD presented in the results section. All antibiotic stock solutions were quality control (QC) tested with E. coli ATCC 25922 prior to experiments and were used only if they produced an MIC result within the accepted CLSI QC range.¹²

MIC determination for individual antimicrobials: Reference broth microdilution (BMD) testing was performed according to CLSI guidelines using the direct colony suspension method.¹³ Serial 2-fold dilutions of each antimicrobial were prepared at double concentrations in 50 µL volumes of CAMHB (BD Diagnostics, Franklin Lakes, NJ) in 96-well plates (Evergreen Scientific, Los Angeles, CA), which were stored at -80° C. until use. Each plate contained negative control wells to assess for contamination of broth or reagents, and positive control wells to verify bacterial growth. A representative plate from each lot was QC tested with E. coli ATCC 25922 prior to use of that lot for clinical strain testing. Maximum antimicrobial concentrations were at least one 2-fold dilution above the resistance breakpoint for Enterobacteriaceae; in the case of rifampin, for which there are no interpretive criteria for Enterobacteriaceae, concentrations up to two 2-fold dilutions above the maximum expected MIC for E. coli ATCC 25922 were included.¹²

Bacterial inocula were prepared by suspending and diluting colonies in CAMHB to an OD₆₀₀ of 0.0006, which corresponds to approximately 1 x 10⁶ cfu/ml for E. coli ATCC 25922. Fifty microliters of the bacterial suspension were added to each well, bringing the bacteria to a final concentration of approximately 5 x 10⁵ cfu/ml. Panels were incubated at 37° C. in ambient air for 16 to 20 hours. The MIC was defined as the lowest concentration of antimicrobial resulting in complete inhibition of growth as determined visually. BMD MICs were determined in duplicate for each strain and antibiotic. If the two results were discrepant, the higher MIC was considered the final BMD MIC.

For fosfomycin, agar dilution reference testing was performed instead, as recommended by CLSI.^{12,}¹³ Agar dilution plates were prepared by adding one part fosfomycin stock solution at ten times the final concentration to nine parts molten Bacto agar (Becton, Dickinson and Company, Sparks, MD) containing non-cation-adjusted Mueller-Hinton broth (Becton, Dickinson and Company, Sparks, MD) and glucose-6-phosphate (G6P; Sigma-Aldrich (St. Louis, MO); final concentration 25 mg/L). At the time of use, bacterial inocula were adjusted to an OD₆₀₀ of 0.01, which corresponds to approximately 1-2 x 10⁷ cfu/ml for E. coli ATCC 25922. Two microliters of this bacterial suspension was spotted on the surface of each agar plate, with each spot containing approximately 1 x 10⁴ cfu. QC testing of E. coli ATCC 25922 was performed in parallel.

DDM MIC testing was performed with the HP D300 digital dispenser (HP, Inc., Palo Alto, CA) as previously described by our laboratory.¹¹ Immediately prior to addition of bacterial suspensions, antimicrobial stock solutions were dispensed by the D300 into empty, flat-bottomed, untreated 384-well polystyrene plates (Greiner Bio-One, Monroe, NC) in volumes ranging from 0.0521 to 323 nL to produce the final desired doubling dilution concentrations with maximum final concentrations at least one 2-fold dilution above the resistance breakpoint for each antibiotic.

Bacterial inocula were adjusted to an OD₆₀₀ of 0.0003 in CAMHB, which corresponds to approximately 5x105 cfu/ml for E. coli ATCC 25922, and 50 µL of this bacterial suspension were added to each well using a multichannel pipette. For fosfomycin testing, the bacterial suspension was supplemented with 25 mg/L G6P. Plates were incubated at 37° C. in ambient air for 16 to 20 hours. After incubation, bacterial growth was quantified by measurement of OD₆₀₀ using an Epoch (BioTek, Winooski, VT) or Spark 10 M microplate reader (Tecan, Morrisville, NC). An OD₆₀₀ reading of 0.08 or greater (approximately twice typical background readings in wells containing broth alone) was considered indicative of bacterial growth (as also appreciable by visual assessment).

Checkerboard array testing: To create checkerboard arrays, serial 2-fold dilutions of antimicrobial pairings were dispensed in orthogonal titrations by the D300, i.e., two-dimensional DDM. Titrations consisted of up to 7 doubling dilutions for each antibiotic. When an isolate’s MIC was below the resistance breakpoint, the maximum concentration tested was 2 doubling dilutions above the MIC. When the MIC was at or above the resistance breakpoint, the maximum concentration tested was at least one doubling dilution above the resistance breakpoint. Inoculum addition, incubation, and growth determination were performed as described for single antimicrobial DDM.

For wells in which growth was inhibited, a fractional inhibitory concentration (FIC) for each antimicrobial was calculated by dividing the concentration of the antibiotic in the well by the MIC of the antibiotic when tested alone.¹⁴ The FIC index (FIC_{I}) was determined by summing the FICs of the two antimicrobials in each inhibited well. The lowest FIC_{I} value in each checkerboard array (FIC_{I-MIN}) was used to determine whether the combination was synergistic, as described below. When a “skipped well” occurred (i.e., inhibition of bacterial growth at a given FIC_{I} but growth at the next highest FIC_{I}), the higher FIC_{I} was considered the FIC_{I-MIN} in order to avoid false positive synergy interpretations.

CLSI-recommended interpretive breakpoints for Enterobacteriaceae were used for all categorical interpretations¹² with the exception of colistin, for which EUCAST breakpoints were used,¹⁵ and rifampin, for which formal interpretive criteria are not available and for which an MIC of ≥4 mg/L was considered resistant in accordance with previous investigations in Acinetobacter species.^{16,}¹⁷

Data analysis: Statistical analysis was performed using R software v3.1 (R Foundation, Vienna, Austria) and Microsoft Excel 2013 (Microsoft Corporation, Redmond, WA). The chi-squared test was used for comparison of proportions.

### Results

Precision and accuracy. Overall, 1585 MIC values were collected during the two stages of DDM synergy testing described below, 78.7% were on-scale (i.e., they fell within the range of MICs included in the antibiotic titration). Modal DDM MICs were calculated by taking the mode of all DDM MIC measurements obtained for a given antibiotic-strain combination, inclusive of initial MIC titrations and single-antibiotic rows/columns in synergy arrays (Table 5). Among on-scale MIC results for which the modal DDM MIC was also on-scale, 96.8% (1141/1179) were within ±1 two-fold dilution of the modal DDM MICs, indicating high intra-method precision as observed previously.^{9,}¹¹ Among on-scale DDM MIC results for which the reference BMD MIC result determined prior to synergy experiments was also on-scale, 91.3% (1025/1123) were within ±1 two-fold dilution of the BMD result. Among all DDM MIC results from assays in which dilution ranges spanned the susceptible, intermediate, and resistant breakpoints, 89.1% (1048/1580) were in categorical agreement with BMD results. Among all DDM MIC results, the minor (either BMD or DDM result intermediate and the other susceptible or resistant); major (DDM resistant and BMD susceptible); and very major (DDM susceptible and BMD resistant) error rates were 10%, 0.13% and 0.44%, respectively. Consistent with our previous results,^{9,}¹¹ these data showed DDM MIC measurements to be both robustly precise and accurate by generally accepted standards,¹⁸ in this case for a highly antimicrobial-resistant CRE strain set with many MICs lying on a breakpoint, which inherently increases rates of minor categorical disagreement. Our results thereby supported use of the underlying technology in two-dimensional checkerboard testing of CRE.

Synergy testing screen. All two-drug combinations of meropenem, aztreonam, cefepime, colistin, gentamicin, levofloxacin, chloramphenicol, fosfomycin, trimethoprim/sulfamethoxazole, minocycline, and rifampin, as well as the double carbapenem combination of meropenem and ertapenem were initially tested in duplicate against 4 bacterial screening strains (BIDMC 4, BIDMC 5, BIDMC 12A, and BIDMC 15). Trials were repeated with a new inoculum if they were uninterpretable due to multiple skipped wells, or if the MIC of either of the individual drugs was more than one 2-fold dilution above or below the MIC determined by DDM in advance of the synergy experiments. If multiple skipped wells recurred on repeat testing, the combination was not further assayed against that strain.

In total, 521 trials were performed, which included 448 trials comprising 56 antibiotic combinations assayed in duplicate against 4 bacterial strains. Forty-nine of 521 trials (9.3%) were uninterpretable due to multiple skipped wells. Nearly all trials with multiple skipped wells (46/49; 94%) included cefepime and/or fosfomycin, and the rates of multiple skipped wells were significantly higher among trials containing cefepime (29/110, 26%) and fosfomycin (21/94; 22.3%) than among trials containing neither of these antibiotics (4/327; 1.2%, p < 0.001).

For each of the 448 trials used for data analysis, the FIC_{I-MIN} was calculated as described in the materials and methods, and the concentration of each antibiotic at the FIC_{I-MIN} was categorized as susceptible, intermediate, or resistant. Trials for which the FIC_{I-MIN} was ≤0.75 and the concentrations of both antibiotics at the FIC_{I-MIN} were within the susceptible or intermediate category were considered to show potential clinically relevant synergy. The FIC_{I-} _{MIN} cutoff of ≤0.75, which is higher than the traditionally accepted cutoff of ≤0.5 for synergy¹⁹, was chosen for screening in order to increase sensitivity for detection of combinations which might show synergy against bacterial strains other than those used at the screening stage.

Overall, 206/448 trials (46%) had an FIC_{I-MIN} of ≤0.75 and 51/448 (11%) met criteria for potential clinically relevant synergy as listed for each combination in Table 4. No trials demonstrated antagonism (FIC_{I-MIN} >4.0).¹⁹

TABLE 4

Number of trials for each antibiotic combination having FIC_{I}-MIN ≤ 0.75 and demonstrating potential clinically relevant synergy

| Antibiotic 1 Antibiotic 2 | Number of trials (total = 8) with FIC_{I}- MIN 0.75 | Number of trials (total = 8) with FIC_{I}-MIN 0.75 and potential clinically relevant synergy | X = combination met criteria for inclusion in spectrum of activity evaluation. (Combinations with cefepime and/or fosfomycin were excluded due to unreliable results) |
|---|---|---|---|
| Aztreonam Chloramphenicol | 2 | 0 | |
| Aztreonam Colistin | 4 | 0 | |
| Aztreonam Fosfomycin | 3 | 0 | |
| Aztreonam Gentamicin | 3 | 0 | |
| Aztreonam Levofloxacin | 3 | 0 | |
| Aztreonam Rifampin | 3 | 0 | |
| Aztreonam TMP-SMX | 0 | 0 | |
| Cefepime Aztreonam | 3 | 0 | |
| Cefepime Chloramphenicol | 5 | 1 | |
| Cefepime Colistin | 3 | 1 | |
| Cefepime Fosfomycin | 3 | 3 | |
| Cefepime Gentamicin | 4 | 1 | |
| Cefepime Levofloxacin | 2 | 0 | |
| Cefepime Meropenem | 2 | 2 | |
| Cefepime Rifampin | 2 | 0 | |
| Cefepime TMP-SMX | 1 | 1 | |
| Chloramphenicol Colistin | 4 | 2 | X |
| Chloramphenicol Fosfomycin | 6 | 1 | |
| Chloramphenicol Rifampin | 5 | 0 | |
| Colistin Rifampin | 5 | 2 | X |
| Fosfomycin Colistin | 5 | 5 | |
| Fosfomycin Rifampin | 3 | 0 | |
| Gentamicin Chloramphenicol | 3 | 0 | |
| Gentamicin Colistin | 7 | 2 | X |
| Gentamicin Fosfomycin | 5 | 3 | |
| Gentamicin Levofloxacin | 4 | 0 | |
| Gentamicin Rifampin | 4 | 0 | |
| Gentamicin TMP-SMX | 3 | 0 | |
| Levofloxacin Chloramphenicol | 0 | 0 | |
| Levofloxacin Colistin | 8 | 2 | X |
| Levofloxacin Fosfomycin | 5 | 0 | |
| Levofloxacin Rifampin | 5 | 0 | |
| Levofloxacin TMP-SMX | 0 | 0 | |
| Meropenem Aztreonam | 5 | 0 | |
| Meropenem Chloramphenicol | 7 | 2 | X |
| Meropenem Colistin | 6 | 2 | X |
| Meropenem Ertapenem | 7 | 0 | |
| Meropenem Fosfomycin | 7 | 4 | |
| Meropenem Gentamicin | 8 | 1 | |
| Meropenem Levofloxacin | 4 | 0 | |
| Meropenem Rifampin | 8 | 1 | |
| Meropenem TMP-SMX | 1 | 0 | |
| Minocycline Aztreonam | 1 | 0 | |
| Minocycline Cefepime | 4 | 2 | |
| Minocycline Chloramphenicol | 5 | 1 | |
| Minocycline Colistin | 6 | 5 | X |
| Minocycline Fosfomycin | 1 | 1 | |
| Minocycline Gentamicin | 2 | 2 | X |
| Minocycline Levofloxacin | 1 | 1 | |
| Minocycline Meropenem | 6 | 2 | X |
| Minocycline Rifampin | 8 | 0 | |
| Minocycline TMP-SMX | 0 | 0 | |
| TMP-SMX Chloramphenicol | 0 | 0 | |
| TMP-SMX Colistin | 1 | 1 | |
| TMP-SMX Fosfomycin | 1 | 0 | |
| TMP-SMX Rifampin | 1 | 0 | |

Spectrum of activity evaluation. The 9 antibiotic combinations that met criteria for potential clinically relevant synergy in 2 or more trials in the screening stage were selected for activity spectrum evaluation. These were minocycline and colistin; colistin and rifampin; gentamicin and colistin; minocycline and gentamicin; levofloxacin and colistin; meropenem and colistin; minocycline and meropenem; chloramphenicol and colistin; and chloramphenicol and meropenem. Combinations containing fosfomycin and/or cefepime were excluded based on the high rates of skipped wells. Each selected combination was tested in duplicate on separate days against the 10 clinical KPC-producing CRE strains (5 K. pneumoniae and 5 E. coli) listed in Table 5, including the original 4 used in the synergy testing screen.

TABLE 5

Characteristics and MICs of carbapenem-resistant bacterial isolates examined in synergy experiments

| Strain Characteristics | | | DDM modal MICs in mg/L | | | | | | | | | | | |
|---|---|---|---|---|---|---|---|---|---|---|---|---|---|---|
| BIDM C Strain | Speci es | KP C typ e | ME M | ET P | AT M | FE P | CS T | MI N | GE N | CH L | LV X | SXT | FO F | RI _{F}^{a} |
| 4 | KPN | 3 | 32 | 32 | >64 | 64 | 0.2 5 | 8 | 32 | >25 6 | 128 | 4/76 | 128 | 32 |
| 5 | KPN | 3 | 8 | 16 | >12 8 | 16 | 0.5 | 16 | 32 | >25 6 | >64 | >8/15 2 | 8 | 32 |
| 10 | KPN | 3 | 4 | 16 | >51 2 | 16 | 0.2 5 | 4 | 32 | >64 | 16 | >32/6 08 | 64 | 32 |
| 12A | KPN | 3 | 8 | 32 | >51 2 | 16 | 0.2 5 | 4 | 1 | 64 | 32 | >8/15 2 | 8 | 32 |
| 18A | KPN | 2 | 32 | 64 | >12 8 | >6 4 | >3 2 | 2 | 64 | >25 6 | 64 | >8/15 2 | 4 | 16 |
| 6 | ECO | 2 | 2 | 8 | 256 | 8 | 0.2 5 | 1 | >32 | 8 | 16 | >32/6 08 | 0.5 | 16 |
| 9 | ECO | 2 | 4 | 8 | >51 2 | 32 | 0.2 5 | 1 | >32 | 8 | 16 | >32/6 08 | 0.5 | 16 |
| 15 | ECO | 2 | 4 | 32 | 512 | 4 | 0.2 5 | 1 | 64 | 8 | 32 | >8/15 2 | 0.5 | 32 |
| 17A | ECO | 2 | 2 | 8 | 256 | 4 | 0.2 5 | 1 | >32 | 8 | 16 | >32/6 08 | 0.5 | 32 |
| 20A | ECO | 3 | 1 | 4 | 256 | 8 | 0.2 5 | 4 | 0.5 | 16 | 32 | >32/6 08 | 1 | 16 |

Underlined text in Table 5 indicates an MIC classified by CLSI or EUCAST (for colistin) as resistant; bold text, intermediate (susceptible dose-dependent for cefepime); and unmarked text, susceptible. Table 5 abbreviations: DDM, digital dispensing inkjet method; KPN, Klebsiella pneumoniae; ECO, Escherichia coli; KPC type, Klebsiella pneumoniae carbapenemase type; MEM, meropenem; ETP, ertapenem; ATM, aztreonam; FEP, cefepime; CST, colistin; MIN, minocycline; GEN, gentamicin; CHL, chloramphenicol; LVX, levofloxacin; SXT, trimethoprim/sulfamethoxazole; FOF, fosfomycin; RIF, rifampin. ^{a}There are no established Enterobacteriaceae interpretive criteria for rifampin. Rifampin MICs ≥4 are classified as resistant as discussed in the text.

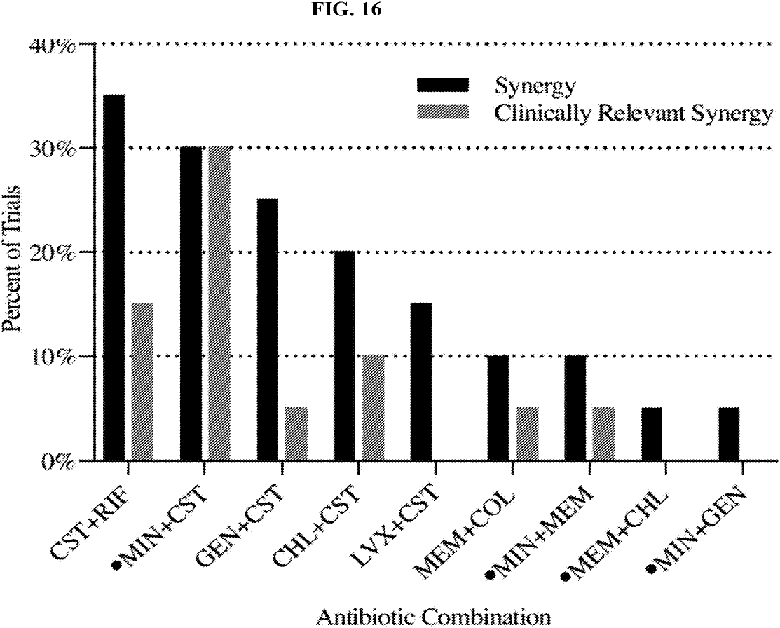

A trial was classified as demonstrating synergy if it had an FIC_{I}-_{MIN} of ≤0.5¹⁹, and clinically relevant synergy if concentrations of both antibiotics at the FIC_{I}-_{MIN} were also within the susceptible or intermediate category. Overall, 31/180 trials (17.2%) demonstrated synergy and 14/180 trials (7.8%) demonstrated clinically relevant synergy. The percent of trials that demonstrated synergy and clinically relevant synergy varied among the antibiotic combinations, with the combination of colistin plus minocycline demonstrating the highest rate of clinically relevant synergy at 30% (FIG. 16). For 8 of the 10 strains (80%), combinations were identified that demonstrated clinically relevant synergy in at least one trial, but these combinations varied among strains (Table 6). The results of all 180 trials are detailed in Table 7.

In 120/180 trials (67%) at least one of the antibiotics had an MIC in the resistant range for the isolate being tested. Clinically relevant synergy was demonstrated in 8/120 (7%) of these cases. In other words, the concentrations of the antibiotic(s) with resistant MICs were brought into the intermediate or susceptible range. These 8 trials represent 57% of the 14 total trials with clinically relevant synergy. In the other 6 trials, both antibiotics had MICs in the susceptible or intermediate range individually. Notably, for strain BIDMC 18A, which is highly resistant to colistin (MIC >128 mg/L), the combinations of colistin plus minocycline and colistin plus rifampin resulted in reduction of inhibitory colistin concentrations into the susceptible range (1-2 mg/L) and similarly dramatic reduction in minocycline and rifampin inhibitory concentrations (0.5 mg/L and 1 mg/L), respectively.

TABLE 6

Antibiotic combinations demonstrating clinically relevant synergy against a CRE strain set

| | | | | | | | |
|---|---|---|---|---|---|---|---|
| BIDMC Isolate | Species | CST+ RIF | •MIN + CST | GEN + CST | CHL + CST | MEM + COL | •MIN + MEM |
| 4 | KPN | | | | | | |
| 5 | KPN | | | | | X | |
| 10 | KPN | | | X | | | |
| 12A | KPN | | | | | | |
| 18A | KPN | X | X | | | | |
| 6 | ECO | | X | | | | |
| 9 | ECO | | | | X | | X |
| 15 | ECO | | X | | | | |
| 17A | ECO | X | X | | | | |
| 20A | ECO | | X | | X | | |

For Table 6: “X” indicates a combination that demonstrated clinically relevant synergy in at least one trial for designated isolate. CST, colistin; RIF, rifampin; MEM, meropenem; MIN, minocycline; GEN, gentamicin; CHL, chloramphenicol; LVX, levofloxacin; KPN = Klebsiella pneumoniae; ECO = Escherichia coli; CRE = carbapenem-resistant Enterobacteriaceae. • identifies a combination for which synergy testing against CRE has not previously been reported.

TABLE 7

Number of trials for each antibiotic combination having FICI-MIN ≤ 0.75 and demonstrating potential clinically relevant synergy

| Strain | Species | Antibiotic 1 | Antibiotic 2 | FIC_{I}- MIN | MIC factor abx 1 at FIC_{I-MI} N^{b} | MIC factor abx 2 at FIC_{I-MI} N^{b} | Conc abx 1 at FIC_{I-MI} N^{c} | Conc abx 2 at FIC_{I-MI} N^{c} | Initia l S/I/R^{d},^{e} | Fina l S/I/ R^{d} |
|---|---|---|---|---|---|---|---|---|---|---|
| BIDMC 15 | E. coli | Chloramphenic ol | Colistin | 0.625 | 0.125 | 0.5 | 1 | 0.125 | S/S | S/S |
| BIDMC 15 | E. coli | Chloramphenic ol | Colistin | 0.625 | 0.125 | 0.5 | 1 | 0.125 | S/S | S/S |
| BIDMC 17A | E. coli | Chloramphenic ol | Colistin | 0.625 | 0.5 | 0.125 | 4 | 0.0625 | S/S | S/S |
| BIDMC 17A | E. coli | Chloramphenic ol | Colistin | 0.75 | 0.25 | 0.5 | 2 | 0.125 | S/S | S/S |
| BIDMC 20A | E. coli | Chloramphenic ol | Colistin | 0.5625 | 0.0625 | 0.5 | 1 | 0.125 | I/S | S/S |
| BIDMC 20A | E. coli | Chloramphenic ol | Colistin | 0.375 | 0.125 | 0.25 | 1 | 0.125 | S/S | S/S |
| BIDMC 6 | E. coli | Chloramphenic ol | Colistin | 0.5625 | 0.5 | 0.0625 | 8 | 0.0625 | I/S | S/S |
| BIDMC 6 | E. coli | Chloramphenic ol | Colistin | 0.75 | 0.5 | 0.25 | 4 | 0.0625 | S/S | S/S |
| BIDMC 9 | E. coli | Chloramphenic ol | Colistin | 0.5 | 0.25 | 0.25 | 4 | 0.0625 | I/S | S/S |
| BIDMC 9 | E. coli | Chloramphenic ol | Colistin | 0.625 | 0.5 | 0.125 | 4 | 0.0625 | S/S | S/S |
| BIDMC 10 | K. pneumoniae | Chloramphenic ol | Colistin | N/A^{a} | | | | | R/S | |
| BIDMC 10 | K. pneumoniae | Chloramphenic ol | Colistin | N/A | | | | | R/S | |
| BIDMC 12A | K. pneumoniae | Chloramphenic ol | Colistin | 1 | 0.5 | 0.5 | 64 | 0.125 | R/S | R/S |
| BIDMC 12A | K. pneumoniae | Chloramphenic ol | Colistin | 1.0625 | 1 | 0.0625 | 64 | 0.0156 | R/S | R/S |
| BIDMC 18A | K. pneumoniae | Chlorampheni col | Colistin | 0.1328 | 0.125 | 0.0078 | 64 | 2 | R/R | R/S |
| BIDMC 18A | K. pneumoniae | Chlorampheni col | Colistin | 0.1328 | 0.125 | 0.0078 | 64 | 2 | R/R | R/S |
| BIDMC 4 | K. pneumoniae | Chloramphenic ol | Colistin | 1 | 0.5 | 0.5 | 128 | 0.25 | R/S | R/S |
| BIDMC 4 | K. pneumoniae | Chloramphenic ol | Colistin | N/A | | | | | R/S | |
| BIDMC 5 | K. pneumoniae | Chloramphenic ol | Colistin | N/A | | | | | R/S | |
| BIDMC 5 | K. pneumoniae | Chloramphenic ol | Colistin | N/A | | | | | R/S | |
| BIDMC 15 | E. coli | Colistin | Rifampin | 0.625 | 0.5 | 0.125 | 0.0625 | 2 | S/R | S/S |
| BIDMC 15 | E. coli | Colistin | Rifampin | 0.75 | 0.25 | 0.5 | 0.031 | 16 | S/R | S/R |
| BIDMC 17A | E. coli | Colistin | Rifampin | 0.5 | 0.25 | 0.25 | 0.0625 | 4 | S/R | S/R |
| BIDMC 17A | E. coli | Colistin | Rifampin | 0.3125 | 0.25 | 0.0625 | 0.0625 | 2 | S/R | S/S |
| BIDMC 20A | E. coli | Colistin | Rifampin | 0.5625 | 0.5 | 0.0625 | 0.0625 | 1 | S/R | S/S |
| BIDMC 20A | E. coli | Colistin | Rifampin | 0.625 | 0.125 | 0.5 | 0.0156 | 16 | S/R | S/R |
| BIDMC 6 | E. coli | Colistin | Rifampin | 0.625 | 0.5 | 0.125 | 0.0625 | 2 | S/R | S/S |
| BIDMC 6 | E. coli | Colistin | Rifampin | 0.75 | 0.5 | 0.25 | 0.125 | 4 | S/R | S/R |
| BIDMC 9 | E. coli | Colistin | Rifampin | 0.5 | 0.25 | 0.25 | 0.031 | 8 | S/R | S/R |
| BIDMC 9 | E. coli | Colistin | Rifampin | 0.5 | 0.25 | 0.25 | 0.0625 | 8 | S/R | S/R |
| BIDMC 10 | K. pneumoniae | Colistin | Rifampin | 0.5 | 0.25 | 0.25 | 0.125 | 8 | S/R | S/R |
| BIDMC 10 | K. pneumoniae | Colistin | Rifampin | 0.531 | 0.5 | 0.031 | 0.125 | 1 | S/R | S/S |
| BIDMC 12A | K. pneumoniae | Colistin | Rifampin | 0.625 | 0.5 | 0.125 | 0.125 | 4 | S/R | S/R |
| BIDMC 12A | K. pneumoniae | Colistin | Rifampin | 0.625 | 0.5 | 0.125 | 0.125 | 4 | S/R | S/R |
| BIDMC 18A | K. pneumoniae | Colistin | Rifampin | 0.0703 1 | 0.00781 | 0.0625 | 2 | 1 | R/R | S/S |
| BIDMC 18A | K. pneumoniae | Colistin | Rifampin | 0.0935 | 0.031 | 0.0625 | 1 | 1 | R/R | S/S |
| BIDMC 4 | K. pneumoniae | Colistin | Rifampin | 1 | 0.5 | 0.5 | 0.125 | 16 | S/R | S/R |
| BIDMC 4 | K. pneumoniae | Colistin | Rifampin | 0.75 | 0.5 | 0.25 | 0.125 | 8 | S/R | S/R |
| BIDMC 5 | K. pneumoniae | Colistin | Rifampin | 0.625 | 0.5 | 0.125 | 0.125 | 4 | S/R | S/R |
| BIDMC 5 | K. pneumoniae | Colistin | Rifampin | 1 | 0.5 | 0.5 | 0.125 | 16 | S/R | S/R |
| BIDMC 15 | E. coli | Gentamicin | Colistin | 0.625 | 0.125 | 0.5 | 4 | 0.25 | R/S | S/S |
| BIDMC 15 | E. coli | Gentamicin | Colistin | 0.75 | 0.5 | 0.25 | 16 | 0.0625 | R/S | R/S |
| BIDMC 17A | E. coli | Gentamicin | Colistin | 0.5 | 0.25 | 0.25 | 32 | 0.0625 | R/S | R/S |
| BIDMC 17A | E. coli | Gentamicin | Colistin | 0.5 | 0.25 | 0.25 | 32 | 0.0625 | R/S | R/S |
| BIDMC 20A | E. coli | Gentamicin | Colistin | 0.625 | 0.5 | 0.125 | 0.25 | 0.031 | S/S | S/S |
| BIDMC 20A | E. coli | Gentamicin | Colistin | 1.0625 | 1 | 0.0625 | 0.25 | 0.0156 | S/S | S/S |
| BIDMC 6 | E. coli | Gentamicin | Colistin | 0.5 | 0.25 | 0.25 | 16 | 0.0625 | R/S | R/S |
| BIDMC 6 | E. coli | Gentamicin | Colistin | 0.75 | 0.5 | 0.25 | 32 | 0.0625 | R/S | R/S |
| BIDMC 9 | E. coli | Gentamicin | Colistin | 0.5 | 0.25 | 0.25 | 16 | 0.0625 | R/S | R/S |
| BIDMC 9 | E. coli | Gentamicin | Colistin | 0.5625 | 0.5 | 0.0625 | 32 | 0.0156 | R/S | R/S |
| BIDMC 10 | K. | Gentamicin | Colistin | 0.375 | 0.125 | 0.25 | 8 | 0.125 | R/S | I/S |
| BIDMC 10 | pneumoniae K. pneumoniae | Gentamicin | Colistin | 1 | 0.5 . | 0.5 | 16 | 0.125 | R/S | R/S |
| BIDMC 12A | K. pneumoniae | Gentamicin | Colistin | 0.5625 | 0.0625 | 0.5 | 0.0625 | 0.125 | S/S | S/S |
| BIDMC 12A | K. pneumoniae | Gentamicin | Colistin | 0.75 | 0.25 | 0.5 | 0.25 | 0.25 | S/S | S/S |
| BIDMC 18A | K. pneumoniae | Gentamicin | Colistin | N/A | | | | | R/R | |
| BIDMC 18A | K. pneumoniae | Gentamicin | Colistin | N/A | | | | | R/R | |
| BIDMC 4 | K. pneumoniae | Gentamicin | Colistin | 0.625 | 0.5 | 0.125 | 16 | 0.031 | R/S | R/S |
| BIDMC 4 | K. pneumoniae | Gentamicin | Colistin | 1 | 0.5 | 0.5 | 16 | 0.125 | R/S | R/S |
| BIDMC 5 | K. pneumoniae | Gentamicin | Colistin | 1 | 0.5 | 0.5 | 16 | 0.125 | R/S | R/S |
| BIDMC 5 | K. pneumoniae | Gentamicin | Colistin | 1 | 0.5 | 0.5 | 16 | 0.125 | R/S | R/S |
| BIDMC 15 | E. coli | Levofloxacin | Colistin | 1 | 0.5 | 0.5 | 8 | 0.125 | R/S | R/S |
| BIDMC 15 | E. coli | Levofloxacin | Colistin | 1 | 0.5 | 0.5 | 8 | 0.25 | R/S | R/S |
| BIDMC 17A | E. coli | Levofloxacin | Colistin | 1 | 0.5 | 0.5 | 16 | 0.125 | R/S | R/S |
| BIDMC 17A | E. coli | Levofloxacin | Colistin | 0.625 | 0.5 | 0.125 | 16 | 0.0156 | R/S | R/S |
| BIDMC 20A | E. coli | Levofloxacin | Colistin | 0.5625 | 0.0625 | 0.5 | 2 | 0.25 | R/S | S/S |
| BIDMC 20A | E. coli | Levofloxacin | Colistin | 0.531 | 0.031 | 0.5 | 1 | 0.25 | R/S | S/S |
| BIDMC 6 | E. coli | Levofloxacin | Colistin | 1 | 0.5 | 0.5 | 8 | 0.125 | R/S | R/S |
| BIDMC 6 | E. coli | Levofloxacin | Colistin | 1 | 0.5 | 0.5 | 8 | 0.125 | R/S | R/S |
| BIDMC 9 | E. coli | Levofloxacin | Colistin | 1.0625 | 1 | 0.0625 | 16 | 0.0156 | R/S | R/S |
| BIDMC 9 | E. coli | Levofloxacin | Colistin | 1 | 0.5 | 0.5 | 8 | 0.125 | R/S | R/S |
| BIDMC 10 | K. pneumoniae | Levofloxacin | Colistin | 0.625 | 0.125 | 0.5 | 2 | 0.125 | R/S | S/S |
| BIDMC 10 | K. pneumoniae | Levofloxacin | Colistin | 1.0625 | 1 | 0.0625 | 16 | 0.0156 | R/S | R/S |
| BIDMC 12A | K. pneumoniae | Levofloxacin | Colistin | 0.75 | 0.25 | 0.5 | 4 | 0.125 | R/S | I/S |
| BIDMC 12A | K. pneumoniae | Levofloxacin | Colistin | 1 | 0.5 | 0.5 | 16 | 0.125 | R/S | R/S |
| BIDMC 18A | K. pneumoniae | Levofloxacin | Colistin | 0.1406 | 0.125 | 0.0156 | 8 | 4 | R/R | R/R |
| BIDMC 18A | K. pneumoniae | Levofloxacin | Colistin | 0.156 | 0.125 | 0.031 | 8 | 8 | R/R | R/R |
| BIDMC 4 | K. pneumoniae | Levofloxacin | Colistin | 0.5 | 0.25 | 0.25 | 16 | 0.125 | R/S | R/S |
| BIDMC 4 | K. pneumoniae | Levofloxacin | Colistin | 0.75 | 0.5 | 0.25 | 32 | 0.0625 | R/S | R/S |
| BIDMC 5 | K. pneumoniae | Levofloxacin | Colistin | 0.625 | 0.125 | 0.5 | 16 | 0.125 | R/S | R/S |
| BIDMC 5 | K. pneumoniae | Levofloxacin | Colistin | 0.75 | 0.25 | 0.5 | 32 | 0.125 | R/S | R/S |
| BIDMC 15 | E. coli | Meropenem | Chloramphenic ol | 0.75 | 0.25 | 0.5 | 1 | 4 | R/S | S/S |
| BIDMC 15 | E. coli | Meropenem | Chloramphenic ol | 0.625 | 0.125 | 0.5 | 0.125 | 8 | S/I | S/S |
| BIDMC 17A | E. coli | Meropenem | Chloramphenic ol | 0.5625 | 0.0625 | 0.5 | 0.125 | 8 | I/S | S/S |
| BIDMC 17A | E. coli | Meropenem | Chloramphenic ol | 0.75 | 0.25 | 0.5 | 0.5 | 4 | I/S | S/S |
| BIDMC 20A | E. coli | Meropenem | Chloramphenic ol | 0.75 | 0.25 | 0.5 | 0.25 | 8 | I/S | S/S |
| BIDMC 20A | E. coli | Meropenem | Chloramphenic ol | 1 | 0.5 | 0.5 | 0.5 | 8 | | S/S |
| BIDMC 6 | E. coli | Meropenem | Chloramphenic ol | 0.625 | 0.5 | 0.125 | 2 | 2 | R/I | I/S |
| BIDMC 6 | E. coli | Meropenem | Chloramphenic ol | 1 | 0.5 | 0.5 | 0.5 | 4 | S/S | S/S |
| BIDMC 9 | E. coli | Meropenem | Chloramphenic ol | 0.5625 | 0.0625 | 0.5 | 0.125 | 8 | I/S | S/S |
| BIDMC 9 | E. coli | Meropenem | Chloramphenic ol | 0.625 | 0.5 | 0.125 | 2 | 1 | R/S | I/S |
| BIDMC 10 | K. pneumoniae | Meropenem | Chloramphenic ol | N/A | | | | | I/R | |
| BIDMC 10 | K. pneumoniae | Meropenem | Chloramphenic ol | N/A | | | | | R/R | |
| BIDMC 12A | K. pneumoniae | Meropenem | Chloramphenic ol | 0.75 | 0.25 | 0.5 | 2 | 64 | R/R | I/R |
| BIDMC 12A | K. pneumoniae | Meropenem | Chloramphenic ol | 0.75 | 0.25 | 0.5 | 4 | 32 | R/R | R/R |
| BIDMC 18A | K. pneumoniae | Meropenem | Chloramphenic ol | 0.5625 | 0.0625 | 0.5 | 2 | 256 | R/R | I/R |
| BIDMC 18A | K. pneumoniae | Meropenem | Chlorampheni col | 0.1875 | 0.125 | 0.0625 | 2 | 256 | R/R | I/R |
| BIDMC 4 | K. pneumoniae | Meropenem | Chloramphenic ol | N/A | | | | | R/R | |
| BIDMC 4 | K. pneumoniae | Meropenem | Chloramphenic ol | N/A | | | | | R/R | |
| BIDMC 5 | K. pneumoniae | Meropenem | Chloramphenic ol | N/A | | | | | R/R | |
| BIDMC 5 | K. pneumoniae | Meropenem | Chloramphenic ol | 0.5156 | 0.5 | 0.0156 | 2 | 32 | R/R | I/R |
| BIDMC 15 | E. coli | Meropenem | Colistin | 0.5625 | 0.0625 | 0.5 | 0.125 | 0.125 | I/S | S/S |
| BIDMC 15 | E. coli | Meropenem | Colistin | 0.75 | 0.5 | 0.25 | 2 | 0.0625 | R/S | ⅟S |
| BIDMC 17A | E. coli | Meropenem | Colistin | 0.5625 | 0.0625 | 0.5 | 0.125 | 0.25 | I/S | S/S |
| BIDMC 17A | E. coli | Meropenem | Colistin | 0.75 | 0.5 | 0.25 | 1 | 0.0625 | I/S | S/S |
| BIDMC 20A | E. coli | Meropenem | Colistin | 0.5625 | 0.0625 | 0.5 | 0.0625 | 0.125 | S/S | S/S |
| BIDMC 20A | E. coli | Meropenem | Colistin | 1.0625 | 1 | 0.0625 | 1 | 0.0156 | S/S | S/S |
| BIDMC 6 | E. coli | Meropenem | Colistin | 0.625 | 0.125 | 0.5 | 0.25 | 0.25 | I/S | S/S |
| BIDMC 6 | E. coli | Meropenem | Colistin | 0.625 | 0.5 | 0.125 | 2 | 0.031 | R/S | I/S |
| BIDMC 9 | E. coli | Meropenem | Colistin | 0.625 | 0.5 | 0.125 | 1 | 0.031 | I/S | S/S |
| BIDMC 9 | E. coli | Meropenem | Colistin | 0.625 | 0.125 | 0.5 | 0.125 | 0.25 | S/S | S/S |
| BIDMC 10 | K. pneumoniae | Meropenem | Colistin | 0.75 | 0.25 | 0.5 | 0.5 | 0.125 | S/S | S/S |
| BIDMC 10 | K. pneumoniae | Meropenem | Colistin | 0.5625 | 0.0625 | 0.5 | 0.25 | 0.25 | R/S | S/S |
| BIDMC 12A | K. pneumoniae | Meropenem | Colistin | 0.531 | 0.031 | 0.5 | 0.25 | 0.125 | R/R | S/S |
| BIDMC 12A | K. pneumoniae | Meropenem | Colistin | 0.75 | 0.25 | 0.5 | 4 | 0.125 | R/S | R/S |
| BIDMC 18A | K. pneumoniae | Meropenem | Colistin | 0.5078 | 0.5 | 0.0078 | 16 | 2 | R/R | R/S |
| BIDMC 18A | K. pneumoniae | Meropenem | Colistin | 0.5078 | 0.5 | 0.0078 | 16 | 2 | R/R | R/S |
| BIDMC 4 | K. pneumoniae | Meropenem | Colistin | 0.5 | 0.25 | 0.25 | 8 | 0.0625 | R/S | R/S |
| BIDMC 4 | K. pneumoniae | Meropenem | Colistin | 0.75 | 0.25 | 0.5 | 8 | 0.125 | R/S | R/S |
| BIDMC 5 | K. pneumoniae pneumoniae | Meropenem | Colistin | 0.5 | 0.25 | 0.25 | 2 | 0.125 | R/S | I/S |
| BIDMC 5 | K. pneumoniae | Meropenem | Colistin | 1 | 0.5 | 0.5 | 2 | 0.125 | R/S | I/S |
| BIDMC 15 | E. coli | Minocycline | Colistin | 0.5 | 0.25 | 0.25 | 0.5 | 0.0625 | S/S | S/S |
| BIDMC 15 | E. coli | Minocycline | Colistin | 1 | 0.5 | 0.5 | 0.5 | 0.0625 | S/S | S/S |
| BIDMC 17A | E. coli | Minocycline | Colistin | 0.5 | 0.25 | 0.25 | 0.25 | 0.125 | S/S | S/S |
| BIDMC 17A | E. coli | Minocycline | Colistin | 0.75 | 0.25 | 0.5 | 0.25 | 0.0625 | S/S | S/S |
| BIDMC 20A | E. coli | Minocycline | Colistin | 0.5 | 0.25 | 0.25 | 1 | 0.0625 | S/S | S/S |
| BIDMC 20A | E. coli | Minocycline | Colistin | 0.531 | 0.031 | 0.5 | 0.125 | 0.125 | S/S | S/S |
| BIDMC 6 | E. coli | Minocycline | Colistin | 0.5 | 0.25 | 0.25 | 0.25 | 0.0625 | S/S | S/S |
| BIDMC 6 | E. coli | Minocycline | Colistin | 0.75 | 0.5 | 0.25 | 0.5 | 0.0625 | S/S | S/S |
| BIDMC 9 | E. coli | Minocycline | Colistin | 0.5625 | 0.0625 | 0.5 | 0.0625 | 0.125 | S/S | S/S |
| BIDMC 9 | E. coli | Minocycline | Colistin | 0.75 | 0.5 | 0.25 | 0.5 | 0.0625 | S/S | S/S |
| BIDMC 10 | K. pneumoniae | Minocycline | Colistin | 1 | 0.5 | 0.5 | 2 | 0.125 | S/S | S/S |
| BIDMC 10 | K. pneumoniae | Minocycline | Colistin | 1.0625 | 0.0625 | 1 | 0.25 | 0.125 | S/S | S/S |
| BIDMC 12A | K. pneumoniae | Minocycline | Colistin | 0.531 | 0.031 | 0.5 | 0.125 | 0.125 | R/S | S/S |
| BIDMC 12A | K. pneumoniae | Minocycline | Colistin | 1 | 0.5 | 0.5 | 2 | 0.125 | S/S | S/S |
| BIDMC 18A | K. pneumoniae | Minocycline | Colistin | 0.1406 | 0.125 | 0.0156 | 0.25 | 4^{f} | S/R | S/S |
| 18A BIDMC 18A | pneumoniae K. pneumoniae | Minocycline | Colistin | 0.156 | 0.125 | 0.031 | 0.25 | 8^{f} | S/R | S/S |
| 18A BIDMC 4 | pneumoniae K. pneumoniae | Minocycline | Colistin | 0.75 | 0.25 | 0.5 | 2 | 0.125 | I/S | S/S |
| BIDMC 4 | K. pneumoniae | Minocycline | Colistin | 1 | 0.5 | 0.5 | 4 | 0.125 | I/S | S/S |
| BIDMC 5 | K. pneumoniae | Minocycline | Colistin | 1 | 0.5 | 0.5 | 4 | 0.125 | I/S | S/S |
| BIDMC 5 | K. pneumoniae | Minocycline | Colistin | 0.625 | 0.5 | 0.125 | 8 | 0.031 | R/S | I/S |
| BIDMC 15 | E. coli | Minocycline | Gentamicin | 1.0625 | 0.0625 | 1 | 0.0625 | 32 | S/R | S/R |
| BIDMC 15 | E. coli | Minocycline | Gentamicin | 1.125 | 0.125 | 1 | 0.125 | 32 | S/R | S/R |
| BIDMC 17A | E. coli | Minocycline | Gentamicin | 0.3125 | 0.0625 | 0.25 | 0.0625 | 32 | S/R | S/R |
| BIDMC 17A | E. coli | Minocycline | Gentamicin | N/A | | | | | S/R | |
| BIDMC 20A | E. coli | Minocycline | Gentamicin | 0.75 | 0.5 | 0.25 | 2 | 0.0625 | S/S | S/S |
| BIDMC 20A | E. coli | Minocycline | Gentamicin | 0.75 | 0.25 | 0.5 | 1 | 0.25 | S/S | S/S |
| BIDMC 6 | E. coli | Minocycline | Gentamicin | 1 | 0.5 | 0.5 | 0.5 | 16 | S/R | S/R |
| BIDMC 6 | E. coli | Minocycline | Gentamicin | 0.5625 | 0.0625 | 0.5 | 0.0625 | 32 | S/R | S/R |
| BIDMC 9 | E. coli | Minocycline | Gentamicin | 1.5 | 0.5 | 1 | 0.5 | 32 | S/R | S/R |
| BIDMC 9 | E. coli | Minocycline | Gentamicin | 0.625 | 0.125 | 0.5 | 0.125 | 32 | S/R | S/R |
| BIDMC 10 | K. pneumoniae | Minocycline | Gentamicin | 1.0625 | 0.0625 | 1 | 0.25 | 32 | S/R | S/R |
| BIDMC 10 | K. pneumoniae | Minocycline | Gentamicin | 0.625 | 0.125 | 0.5 | 0.5 | 32 | S/R | S/R |
| BIDMC 12A | K. pneumoniae | Minocycline | Gentamicin | 0.531 | 0.031 | 0.5 | 0.125 | 1 | S/S | S/S |
| BIDMC 12A | K. pneumoniae | Minocycline | Gentamicin | 1 | 0.5 | 0.5 | 1 | 0.5 | S/S | S/S |
| BIDMC 18A | K. pneumoniae | Minocycline | Gentamicin | N/A | | | | | S/R | |
| BIDMC 18A | K. pneumoniae | Minocycline | Gentamicin | N/A | | | | | S/R | |
| BIDMC 4 | K. pneumoniae | Minocycline | Gentamicin | 1.0625 | 0.0625 | 1 | 0.5 | 32 | I/R | S/R |
| BIDMC 4 | K. pneumoniae | Minocycline | Gentamicin | 1.0625 | 0.0625 | 1 | 0.5 | 32 | I/R | S/R |
| BIDMC 5 | K. pneumoniae | Minocycline | Gentamicin | 1.125 | 0.125 | 1 | 1 | 32 | I/R | S/R |
| BIDMC 5 | K. pneumoniae | Minocycline | Gentamicin | 1.125 | 0.125 | 1 | 1 | 32 | I/R | S/R |
| BIDMC 15 | E. coli | Minocycline | Meropenem | 1 | 0.5 | 0.5 | 0.5 | 1 | S/I | S/S |
| BIDMC 15 | E. coli | Minocycline | Meropenem | 2.125 | 2 | 0.125 | 1 | 0.125 | S/S | S/S |
| BIDMC 17A | E. coli | Minocycline | Meropenem | 0.625 | 0.5 | 0.125 | 1 | 0.25 | I/S | S/S |
| BIDMC 17A | E. coli | Minocycline | Meropenem | 1.125 | 1 | 0.125 | 1 | 0.125 | S/S | S/S |
| BIDMC 20A | E. coli | Minocycline | Meropenem | 1.0625 | 1 | 0.0625 | 2 | 0.0625 | S/S | S/S |
| BIDMC 20A | E. coli | Minocycline | Meropenem | 1.0625 | 1 | 0.0625 | 4 | 0.0625 | S/S | S/S |
| BIDMC 6 | E. coli | Minocycline | Meropenem | 0.625 | 0.5 | 0.125 | 1 | 0.25 | I/S | S/S |
| BIDMC 6 | E. coli | Minocycline | Meropenem | 1.125 | 1 | 0.125 | 1 | 0.125 | S/S | S/S |
| BIDMC 9 | E. coli | Minocycline | Meropenem | 0.5 | 0.25 | 0.25 | 0.5 | 1 | S/R | S/S |
| BIDMC 9 | E. coli | Minocycline | Meropenem | 1.125 | 1 | 0.125 | 1 | 0.125 | S/S | S/S |
| BIDMC 10 | K. pneumoniae | Minocycline | Meropenem | 1.0625 | 1 | 0.0625 | 4 | 0.125 | S/I | S/S |
| BIDMC 10 | K. pneumoniae | Minocycline | Meropenem | 1.0625 | 0.0625 | 1 | 0.25 | 4 | S/R | S/R |
| BIDMC 12A | K. pneumoniae | Minocycline | Meropenem | 1.031 | 1 | 0.031 | 4 | 0.125 | S/R | S/S |
| BIDMC 12A | K. pneumoniae | Minocycline | Meropenem | 1.0625 | 1 | 0.0625 | 2 | 0.5 | S/R | S/S |
| BIDMC 18A | K. pneumoniae | Minocycline | Meropenem | 0.75 | 0.5 | 0.25 | 1 | 8 | S/R | S/R |
| BIDMC 18A | K. pneumoniae | Minocycline | Meropenem | 0.5 | 0.25 | 0.25 | 1 | 8 | S/R | S/R |
| BIDMC 4 | K. pneumoniae | Minocycline | Meropenem | 1.125 | 1 | 0.125 | 8 | 4 | I/R | I/R |
| BIDMC 4 | K. pneumoniae | Minocycline | Meropenem | 1 | 0.5 | 0.5 | 4 | 16 | I/R | S/R |
| BIDMC 5 | K. pneumoniae | Minocycline | Meropenem | 1 | 0.5 | 0.5 | 4 | 8 | I/R | S/R |
| BIDMC 5 | K. pneumoniae | Minocycline | Meropenem | 1.25 | 1 | 0.25 | 8 | 4 | I/R | I/R |

For Table 7:
Rows containing underlined text indicate trials with clinically relevant synergy; rows containing bold text indicate trials with synergy that did not qualify as clinically relevant, as described in the text.
^{a}N/A: the concentrations of one or both agents remained above the range of dilutions tested, even in combination.
^{b}Proportion by which the antibiotic’s individual MIC is reduced at the FIC_{I-MIN}.
^{c}Antibiotic concentration, in µg/mL, at the FIC_{I-MIN}.
^{d}Interpretation of the concentrations of antibiotics 1 and 2 individually (Initial S/I/R) (S, susceptible; I, intermediate; R, resistant) and at the FIC_{I-MIN} (Final S/I/R)
^{e}Initial S/I/R interpretations are taken from wells in the individual trial containing a single antibiotic, so may vary by +/- one 2-fold dilution from the pre-determined MIC; in some instances this results in a change of interpretation.
^{f}At FIC_{I} of 0.2578 (still within range of synergy but above FIC_{I-MIN}) colistin concentration is 2 and minocycline concentration is 0.5.

### Discussion

Through the application of an inkjet printer-based automated checkerboard method, we were able to rapidly test 56 antimicrobial combinations, including many that have not been evaluated in the literature to date, against a screening set of CRE strains. We identified 6 combinations that showed clinically relevant synergy in one or more trials in activity spectrum evaluation. Among these were 2 combinations that have frequently been reported as demonstrating synergy against CRE (colistin plus rifampin^{8,} ^{20,} ²¹ and a carbapenem plus colistin^{8,}22, 23).

Three of our findings merit further discussion. First, we identified 2 novel antibiotic combinations demonstrating clinically relevant synergy whose efficacy against CRE has not previously been described (FIG. 16; minocycline and colistin; minocycline and meropenem). Both of these combinations include minocycline, a tetracycline antibiotic that has recently attracted attention as a therapeutic option for CRE and other resistant Gram-negative bacteria.^{24,} ²⁵ Minocycline has several potential advantages over the tetracycline derivative tigecycline, a glycylcycline antibiotic that has been used to treat CRE both alone and in combination.^{25,} ²⁶ Unlike tigecycline, minocycline is available in both oral and intravenous (IV) forms, allowing for easier outpatient therapy in patients with less severe infections or those for whom a longer course of therapy is desired after completion of an initial IV antibiotic course. Minocycline also has a generally favorable side-effect profile,²⁷ while tigecycline is associated with significant rates of nausea and vomiting.²⁸ Furthermore, unlike tigecycline, which has limited urinary excretion,²⁹ raising concerns about its utility for treatment of urinary tract infection (UTI),^{28,} ³⁰ minocycline has an FDA-approved indication for UTI,³¹ which is one of the most common manifestations of CRE infection. ^{1,} ³² Minocycline is also potentially a preferable agent for treatment of bloodstream infections, as it reaches higher serum concentrations than tigecycline.^{29,31}

Second, while some double carbapenem combinations have previously been shown to demonstrate in vitro synergy against CRE,³³ we did not observe clinically relevant synergy for the combination of meropenem and ertapenem in the screening stage. This may have been due to the high ertapenem MICs of the strains, which ranged from 8 to 64 mg/L. A previous investigation of the combination of meropenem and ertapenem similarly showed high rates of synergy, but at concentrations above those clinically achievable.³⁴ Therefore, data suggest that meropenem-ertapenem combinations may not provide reliable benefit.

Third, significant heterogeneity in synergistic activity was observed against different strains, a finding that is consistent with prior studies, but not generally emphasized.^{5,} ^{8,} ^{35, 37, 38} Even combinations with the highest rates of synergistic activity showed clinically relevant synergy in no more than one-third of trials. Importantly, this finding underscores the need for individualized synergy testing to determine which combinations will be effective for a given patient’s isolate. Studies such as ours, which identify those combinations that are most likely to be synergistic, can be used to select high-yield combinations for clinical testing.

In this study, we classified combinations according to whether or not they demonstrated clinically relevant synergy. We considered combinations in which inhibitory concentrations were lowered into the intermediate range clinically relevant because there is increasing interest in the use of higher doses of antibiotics, particularly β-lactams, to treat organisms with MICs classified as intermediate.³⁷⁻³⁹ The concept of clinically relevant synergy has not been consistently applied in the literature to date, with some studies reporting only synergistic combinations in which concentrations fall within a clinically achievable range based on pharmacodynamic parameters,⁵ while others present only limited data, if any, on the final concentrations of antibiotics in synergistic combinations.^{8,} ³⁵ Furthermore, the clinical significance of synergy in combinations in which an isolate is already susceptible to each antibiotic individually is less well established and warrants further investigation. It is possible, for example, that dosing of some of the most toxic antibiotics, including colistin and aminoglycosides, could be reduced, thus potentially decreasing the risk of toxicity.

In determining which combinations met criteria for synergy in the activity spectrum evaluation, we used the standard FIC_{I-MIN} cutoff of ≤0.5. A conservative cutoff of this type corrects for the well-known +/- one 2-fold variability inherent in MIC testing. ^{18,} ⁴⁰ This variability is inevitably increased when two antibiotics are assayed simultaneously. However, it is plausible that FIC_{I-MIN} values that repeatedly fall just above the 0.5 cutoff are truly synergistic. Notably, the ease with which DDM checkerboard synergy testing can be performed could allow for combinations to be routinely tested in duplicate or triplicate, thereby increasing confidence in the FIC_{I-MIN} result.

Importantly, we demonstrated the utility of a new technology to support systematic testing of a wide range of antibiotic combinations against a collection of CREs. In doing so, we identified novel synergistic combinations and have also illustrated the variability of synergistic activity against different CRE strains. Future studies that expand synergy testing against a comprehensive, diverse collection of CREs will be needed to provide the foundation for a more definitive understanding of the antibiotic combinations that are most frequently synergistic against specific types of CRE, which will in turn serve as guidance for empiric combination therapy and for focused testing of patient isolates. This study demonstrates that DDM technology is useful for synergy checkerboard analysis within an actionable time frame using prospectively or retrospectively collected isolates.

### References for Example 5

1. Temkin E, Adler A, Lerner A et al. Carbapenem-resistant Enterobacteriaceae: Biology, epidemiology, and management. Annals of the New York Academy of Sciences 2014; 1323: 22-42.
2. Zusman O, Avni T, Leibovici L et al. Systematic review and meta-analysis of in vitro synergy of polymyxins and carbapenems. Antimicrob Agents Chemother 2013; 57: 5104-11.
3. Paul M, Carmeli Y, Durante-Mangoni E et al. Combination therapy for carbapenem-resistant Gram-negative bacteria. JAntimicrob Chemother 2014; 69: 2305-9.
4. Rafailidis PI, Falagas ME. Options for treating carbapenem-resistant Enterobacteriaceae. Current opinion in infectious diseases 2014; 27: 479-83.
5. Clock SA, Tabibi S, Alba L et al. In vitro activity of doripenem alone and in multi-agent combinations against extensively drug-resistant Acinetobacter baumannii and Klebsiella pneumoniae. Diagn Microbiol Infect Dis 2013; 76: 343-6.
6. Hirsch EB, Guo B, Chang KT et al. Assessment of antimicrobial combinations for Klebsiella pneumoniae carbapenemase-producing K. pneumoniae. J InfectDis 2013; 207: 786-93.
7. Tängdén T, Hickman RA, Forsberg P et al. Evaluation of double- and triple-antibiotic combinations for VIM- and NDM-producing Klebsiella pneumoniae by in vitro time-kill experiments. Antimicrob Agents Chemother 2014; 58: 1757-62.
8. Tascini C, Tagliaferri E, Giani T et al. Synergistic activity of colistin plus rifampin against colistin-resistant KPC-producing Klebsiella pneumoniae. Antimicrob Agents Chemother 2013; 57: 3990-3.
9. Smith KP, Brennan-Krohn T, Weir S et al. Improved Accuracy of Cefepime Susceptibility Testing for ESBL-producing Enterobacteriaceae using an On-Demand Digital Dispensing Method. J Clin Microbiol 2016.
10. Smith KP, Kirby JE. How inkjet printing technology can defeat multidrug-resistant pathogens. Future microbiology 2016; 11: 1375-7.
11. Smith KP, Kirby JE. Verification of an automated, digital dispensing platform for at-will broth microdilution-based antimicrobial susceptibility testing. J Clin Microbiol 2016; 54: 2288-93.
12. Clinical and Laboratory Standards Institute. Performance Standards For Antimicrobial Susceptibility Testing: Twenty-Sixth Informational Supplement M100-S26. Wayne, PA: CLSI, 2016.
13. Clinical and Laboratory Standards Institute. Methods For Dilution Antimicrobial Susceptibility Tests For Bacteria That Grow Aerobically - Tenth Edition: Approved Standard M07-A10. Wayne, PA: CLSI, 2015.
14. Elion GB, Singer S, Hitchings GH. Antagonists of nucleic acid derivatives. VIII. Synergism in combinations of biochemically related antimetabolites. The Journal of biological chemistry 1954; 208: 477-88.
15. The European Committee on Antimicrobial Susceptibility Testing. Clinical Breakpoints. Available at www.eucast.org/clinical_breakpoints on the World Wide Web.
16. Henwood CJ, Gatward T, Warner M et al. Antibiotic resistance among clinical isolates of Acinetobacter in the UK, and in vitro evaluation of tigecycline (GAR-936). J Antimicrob Chemother 2002; 49: 479-87.
17. Tan TY, Ng LS, Poh K. Susceptibility testing of unconventional antibiotics against multiresistant Acinetobacter spp. by agar dilution and Vitek 2. Diagn Microbiol Infect Dis 2007; 58: 357-61.
18. Clark R, Lewinski M, Loeffelholz M et al. Cumitech 31A, Verification and Validation of Procedures In the Clinical Microbiology Laboratory. Washington, DC: ASM Press, 2009.
19. Odds FC. Synergy, antagonism, and what the chequerboard puts between them. J Antimicrob Chemother 2003; 52: 1-.
20. Bratu S, Tolaney P, Karumudi U et al. Carbapenemase-producing Klebsiella pneumoniae in Brooklyn, NY: Molecular epidemiology and in vitro activity of polymyxin B and other agents. JAntimicrob Chemother 2005; 56: 128-32.
21. Elemam A, Rahimian J, Doymaz M. In vitro evaluation of antibiotic synergy for polymyxin B-resistant carbapenemase-producing Klebsiella pneumoniae. J Clin Microbiol 2010; 48: 3558-62.
22. Jernigan MG, Press EG, Nguyen MH et al. The combination of doripenem and colistin is bactericidal and synergistic against colistin-resistant, carbapenemase-producing Klebsiella pneumoniae. Antimicrob Agents Chemother 2012; 56: 3395-8.
23. Pankey GA, Ashcraft DS. Detection of synergy using the combination of polymyxin B with either meropenem or rifampin against carbapenemase-producing Klebsiella pneumoniae. Diagn Microbiol Infect Dis 2011; 70: 561-4.
24. Pogue JM, Neelakanta A, Mynatt RP et al. Carbapenem-resistance in Gram-negative bacilli and intravenous minocycline: An antimicrobial stewardship approach at the Detroit Medical Center. Clin Infect Dis 2014; 59: S388-S93.
25. Thaden JT, Pogue JM, Kaye KS. Role of newer and re-emerging older agents in the treatment of infections caused by carbapenem-resistant Enterobacteriaceae. Virulence 2016; 0: 1-16.
26. Ni W, Han Y, Liu J et al. Tigecycline treatment for carbapenem-resistant Enterobacteriaceae infections: A systematic review and meta-analysis. Medicine 2016; 95: e3126-e.
27. Ritchie DJ, Garavaglia-Wilson A. A review of intravenous minocycline for treatment of multidrug-resistant Acinetobacter infections. Clin Infect Dis 2014; 59: S374-80.
28. Doan TL, Fung HB, Mehta D et al. Tigecycline: A glycylcycline antimicrobial agent. Clinical Therapeutics 2006; 28: 1079-106.
29. Wyeth Pharmaceuticals. Tygacil® (tigecycline) For Injection, Package Insert. Philadelphia, PA, 2010.
30. Muralidharan G, Micalizzi M, Speth J et al. Pharmacokinetics of tigecycline after single and multiple doses in healthy subjects. Antimicrob Agents Chemother 2005; 49: 220-9.
31. The Medicines Company. MINOCIN® Minocycline For Injection, Package Insert. Parsippany, NJ, 2015.
32. Guh AY, Bulens SN, Mu Y et al. Epidemiology of carbapenem-resistant Enterobacteriaceae in 7 US communities, 2012-2013. JAMA 2015; 314: 1479-87.
33. Poirel L, Kieffer N, Nordmann P. In vitro evaluation of dual carbapenem combinations against carbapenemase-producing Enterobacteriaceae. JAntimicrob Chemother 2016; 71: 156-61.
34. Oliva A, Gizzi F, Mascellino MT et al. Bactericidal and synergistic activity of double-carbapenem regimen for infections caused by carbapenemase-producing Klebsiella pneumoniae. Clin Microbiol Infect 2016; 22: 147-53.
35. Berçot B, Poirel L, Dortet L et al. In vitro evaluation of antibiotic synergy for NDM-1-producing Enterobacteriaceae. JAntimicrob Chemother 2011; 66: 2295-7.
36. Albiero J, Sy SKB, Mazucheli J et al. Pharmacodynamic evaluation of the potential clinical utility of fosfomycin and meropenem in combination therapy against KPC-2-producing Klebsiella pneumoniae. Antimicrob Agents Chemother 2016; 60: 4128-39.
37. Morrill HJ, Pogue JM, Kaye KS et al. Treatment options for carbapenem-resistant Enterobacteriaceae infections. Open Forum Infectious Diseases 2015; 2.
38. Daikos GL, Markogiannakis A. Carbapenemase-producing Klebsiella pneumoniae: (When) might we still consider treating with carbapenems? Clin Microbiol Infect 2011; 17: 1135-41.
39. Alves MD, Ribeiro VB, Tessari JP et al. Effect of cefepime dose on mortality of patients with Gram-negative bacterial bloodstream infections: A prospective cohort study. J Antimicrob Chemother 2014; 69: 1681-7.
40. U. S. Food and Drug Administration. Class II Special Controls Guidance Document: Antimicrobial Susceptibility Test (AST) Systems. Rockville, MD, 2009.

### Example 6: Microscopy-Based AST Platform (MAST)

The rapid emergence of antimicrobial resistance presents a significant challenge for treatment of bacterial infection. To guide appropriate therapy and preserve efficacy of existing antimicrobials, hospital-based clinical microbiology laboratories perform antimicrobial susceptibility testing (AST) of pathogen isolates to determine which drugs will be active against an infecting organism. The gold standard methods used for AST require manual preparation of an antimicrobial doubling dilution series in agar or broth that is tested for antimicrobial effect and minimal inhibitory concentration (MIC) determination. MIC values are defined as the lowest concentration of antimicrobial that results in visible growth inhibition of an organism after 16-20 hours of incubation.¹ MIC results are interpreted based on consensus guidelines to classify the pathogen as susceptible (S), intermediate (I), or resistant (R) to the tested antimicrobial.²

However, technical complexity of manual dilution testing, even for a few isolates or drugs, precludes use of these reference methods in hospital-based clinical laboratories.³ Therefore, alternative testing methods are typically used including various types of pre-formulated antimicrobial dilution panels such as Sensititre (ThermoFisher, Waltham, MA); methods that extrapolate MICs based on growth kinetics in the presence of a limited number of different antimicrobial concentrations, such as Vitek 2 (bioMerieux, Durham, NC); and diffusion based methods that can predict MIC-based susceptibility categories for a subset of organism-drug combinations.³ However, these tests are limited in that they incorporate only predetermined panels of relatively common antimicrobials; do not include recently approved agents developed for multidrug-resistant pathogens;⁴ require extended (>8-20 hour) incubation; and for MIC surrogate methods, may not always correlate with reference MIC results, a problem particularly noted for multidrug-resistant organisms.⁵⁻⁶

This example describes the inventors’ multi-component, microscopy-based AST platform (MAST) which is capable of AST determinations after only a 2 hour incubation. In particular, this example describes development and validation of a platform that includes a solid-phase microwell growth surface in a 384-well plate format, inkjet printing-based dispensing of antimicrobials and bacteria at any desired concentrations, automated microscopic imaging of bacterial replication, and a deep learning approach for automated image classification and determination of antimicrobial minimal inhibitory concentrations. As described in the paragraphs that follow, we evaluated a susceptible strain set and determined that 95.8% and 99.4% of our MAST MIC results were within ±1 or ±2, two-fold dilutions of reference broth microdilution MIC values, respectively. 98.3% of results were in categorical agreement, thus demonstrating that MAST offers a platform for rapid, accurate, and flexible AST to help address the significant antimicrobial testing gap in which current methodologies cannot adequately address the need for rapid results in the face of unpredictable susceptibility profiles.

### Materials and Methods

Bacterial strains and antimicrobials. Escherichia coli ATCC 25922, Enterobacter cloacae ATCC 13047, Klebsiella pneumoniae ATCC 13883, Pseudomonas aeruginosa ATCC 27853, and Acinetobacter baumannii 17978 were obtained from the American Type Culture Collection (Manassas, VA). Strains were stored at -80° C. in tryptic soy broth (BD Diagnostics, Franklin Lakes, NJ) containing 50% glycerol (Sigma-Aldrich, St. Louis, MO).

Meropenem was from Ark Pharm (Arlington Heights, IL), cefepime was from Chem Impex (Wood Dale, IL), gentamicin was from Alfa Aesar (Haverhill, MA), and ciprofloxacin was from US Biological (Salem, MA). Antibiotic stock solutions were prepared in water for manual dilution-based testing or in water containing 0.3% polysorbate-20 as required for liquid handling by the HP D300 digital dispenser (HP Inc., Palo Alto, CA).⁸ We previously determined through extensive analysis that polysorbate-20 at the concentrations used in assay wells has no effect on MIC determinations for all antibiotics examined.^{5,} ⁹

Broth microdilution (BMD) susceptibility testing. BMD was performed according to Clinical Laboratory and Standards Institute (CLSI) guidelines.¹ Specifically, serial two-fold dilutions of antimicrobials were prepared in cation-adjusted Mueller Hinton broth (CAMHB, BD Diagnostics) in sterile, polystyrene 96-well plates (Evergreen Scientific, Los Angeles, CA) in a 50 µl volume. Bacteria were grown overnight at 35±2° C. in ambient air on tryptic soy agar containing 5% sheep’s blood. Several colonies were suspended in sterile 0.9% saline (Sigma-Aldrich) to a density corresponding to 0.5 McFarland as measured using a DensiCHEK Plus Colorimeter (bioMerieux, Durham, NC). This suspension was diluted 1:150 in sterile CAMHB, and 50 µl was added to antibiotic-containing wells resulting in an inoculum density of approximately 5 × 10⁵ colony forming units (CFU) ml⁻¹ in a 100 µl assay volume. Inoculated plates were incubated at 35±2° C. in ambient air for 16-20 hours. Minimal inhibitory concentration (MIC) was defined as the lowest antimicrobial concentration resulting in complete inhibition of growth as determined visually.¹ BMD experiments were performed in triplicate for each antibiotic/organism combination with the modal result recorded as the reference MIC.⁵

Preparation of solid microwell surfaces in assay plates. In preliminary experiments, we evaluated several agents for solidifying CAMHB including BD Bacto agar (1.5% w/v, BD Diagnostics), gellan gum (1% w/v, trade name GELRITE, Research Products International, Mt. Prospect, IL), polyacrylamide (15% v/v, ThermoFisher Scientific, Waltham, MA) and poloxamer 407 (15% w/v, trade name Pluronic F-127, Sigma-Aldrich). We identified 15% poloxamer 407 as the ideal solidifying agent, and all future experiments were therefore conducted with 15% poloxamer 407 in CAMHB (hereafter called CAMHB-P). Prior to use, CAMHB-P was centrifuged at 4,000 × g at 4° C. for 10 minutes to remove small particles of media/poloxamer 407 which may interfere with microscopy. The cleared solution was kept on ice, and 10 µl was added to wells of clear polystyrene 384-well plates (Greiner Bio-One, Monroe, NC). Immediately after preparation, plates were centrifuged at 3,500 × g at 4° C. for 5 minutes to ensure complete coverage of wells with CAMHB-P and stored at -80° C. until use.

Digital dispensing of bacteria. Prior to dispensing, organisms were grown for 16-20 hours at 35±2° C. in ambient air on tryptic soy agar containing 5% sheep’s blood. Several colonies were suspended in 0.9% NaCl containing 0.3% polysorbate-20 (NaCl—P20) and adjusted to the desired density using a handheld colorimeter. Suspensions were directly loaded into D4+ or T8+ cassettes (HP Inc.), and dispensed into wells of 384-well plates at volumes indicated using the HP D300 Digital Dispenser (HP Inc.).

Quantification of bacterial dispensing precision. Precision of bacterial inkjet dispensing was quantified by two complimentary methods: colony forming unit (CFU) determination and microscopic cell counting. For CFU quantification, 50, 150, 250, and 350 nL of a 0.5±0.05 McFarland suspension of bacteria in NaCl-P20 were dispensed into wells of a 384-well plate containing 50 µl of CAMHB. The inoculated media was diluted 1:1000 in sterile CAMHB, and 100 µl was plated on Mueller-Hinton plates (Remel, Lenexa, KS). Plates were incubated for 16-20 hours at 37° C. in ambient air, and colonies were counted to determine CFU/ml according to CLSI guidelines for inoculum density determination.¹ Each experiment was performed in triplicate with three wells sampled per experiment. Standard curves comparing dispense volume to bacterial density (CFU/ml) were generated using a custom Python (version 3.5.0) script, Matplotlib (version 2.0.0)¹⁰ and NumPy (version 1.11.3).¹¹

For quantification by direct imaging, a 0.5 McFarland suspension in NaCl-P20 was diluted 1:5, and 200 nL was dispensed into the center of each of 240 wells of a 384-well plate containing CAMHB-P. The outer two rows of wells were not used to avoid edge effects resulting from evaporative loss. Plates were kept at room temperature until imaging at the Harvard Center for Biological Imaging (HCBI, Harvard University, Cambridge, MA) using a Zeiss Cell Observer microscope (Zeiss, Oberkochen, Germany) operating in brightfield mode with a 40X air objective (0.6 NA, 2.9 mm working distance) and an automated mechanical stage. The x and y position of each well was determined automatically using a pre-loaded plate map. Z-position for optimal focus was adjusted manually. A single field corresponding to the center of each well was imaged as a z-series of 40 µm with a step size of 2 µm. Image stacks were projected using the extended depth of focus module within the Zeiss Zen Blue software. Individual bacteria in resulting images were manually segmented and counted using the particle analysis function in ImageJ.¹² Twelve wells were imaged in each of three independent experiments. A bar graph was generated using a custom Python script (version 3.5.0) and the Matplotlib library.¹⁰

Microscopy-based AST (MAST). Antimicrobials were applied by digital dispensing using the HP D300, as previously described,⁹ into 384-well plates (Greiner Bio-One) equilibrated to 35±2° C. containing 10 µl of solid CAMHB-P. Final antimicrobial concentrations in solidified wells ranged from 0.004-1 µg/ml for ciprofloxacin and meropenem, 0.016-4 µg/ml for cefepime, and 0.03-8 µg/ml for gentamicin. Two-hundred nL of bacteria in NaCl-P20 suspended at a density of 0.1 McFarland were then delivered to the center of each well by digital dispensing. Immediately after inoculation, plates were incubated at 35±2° C. for 2 hours, and held at room temperature for 2 hours during transport and imaging.

Plates were imaged with a Zeiss Cell Observer microscope with settings described in the “quantification of bacterial dispensing” section. Following manual selection of the z-position for the first well in the series, the microscope stage independently advanced to subsequent wells based on a plate map with the z-position updated automatically using the autofocus feature of the microscope control software (Zeiss Zen Blue software version 2.1 and 2.3). For each well, a z-series of 40 µm (step size = 2 µm) was automatically collected and projected to form a single image using the extended depth of focus module in the Zeiss Zen Blue software (version 2.1 and 2.3). Custom Python scripts were used to export individual images as .jpeg files with the appropriate organism name, antibiotic concentration, and well location encoded in the filename to automate downstream deep learning analysis.

Image classification by deep learning. Six replicate dilution series for each antibiotic/organism combination were imaged in three independent MAST experiments yielding 3,202 training images. Each image was manually classified as “growth” or “inhibition” based on known morphological characteristics of growing or inhibited cells.¹³⁻¹⁵ Each annotated image had dimensions 2048x2048 px, and was cropped into 64 non-overlapping 256 x256 px images, which were further augmented by random blur and rotation to generate 512 augmented images per annotated image, for a total of 1,639,424 training images. 80% of these data were used for training a convolutional neural network (ConvNet), and 20% of these data were used for validation. During training, each 256 x256 px image was additionally augmented ‘on the fly’ by random crop to generate a 220x220 px input image for the network.

The ConvNet architecture followed the VGG-style¹⁶ with small (3x3 pixel) receptive fields, stacked convolutional kernels with stride and pad equal to 1 pixel. Referring to FIG. 23, convolutional network 30 comprises eight convolutional layers 32, three fully connected layers 34, and four max pool layers 36. The number of feature maps were increased by a factor of two after every spatial pooling layer, resulting in an overall bi-pyramidal architecture. All convolutional layers were batch normalized, with learned scale and shift, followed by a rectified linear unit (ReLU) non-linearity. The first two fully connected layers were regularized by drop-out, with a drop-out probability of 0.5. The final activation was a 2-way softmax, corresponding to the categories “growth” and “inhibition.” ConvNet 30 receives “original” or unaltered input image I₀ 38.

Training was done using mini-batch stochastic gradient descent (batch size 32), based on backpropagation with momentum. The loss function was cross-entropy, with additional regularization in the form of L2 weight decay. All networks were trained from random initializations using the Xavier initialization scheme (Xavier Glorot and Yoshua Bengio, Understanding the difficulty of training deep feedforward neural networks. AISTATS, 2010). The initial learning rate was set to 0.0003, and decayed according to an inverse schedule. Momentum was 0.9, and weight decay was 0.0001.

For the purposes of evaluating whole images, all image crops for a given image were evaluated to calculate three parameters: mean image inhibition probability; median image inhibition probability, and proportion of crops with inhibition probability >0.5.

Optimization of MIC calls. To optimize our ability to call MICs based on ConvNet output, we collected a separate dataset, independent of the training dataset, which we refer to as the optimization dataset. We used the MAST assay to collect images from all combinations of antibiotics and organisms listed in “Bacterial strains and antimicrobials” on three separate days (180 MIC assays x 10 antibiotic concentrations = 1800 images). Images in the optimization dataset were evaluated using our trained ConvNet and all output parameters were recorded (mean image inhibition probability, median inhibition probability, and proportion of crops with inhibition probability >0.5).

A custom Python script was used to model results from each dilution series as a sigmoidal curve using each of the three output parameters. For the dataset comprising antibiotic/parameter combinations, we iteratively set a threshold (ranging between 0 and 0.99), which represented the point on the sigmoidal curve above which all results would be called “inhibited.” We then calculated MIC accuracy at each threshold and identified the optimal parameter and threshold combination which resulted in the highest MIC accuracy. If the highest accuracy were achieved at multiple potential threshold values for a given parameter, the median of those values was designated as the optimal threshold cutoff value.

Proof-of-principle application of MAST assay for end-to-end MIC determination. A verification dataset, independent of both the training and optimization datasets, was collected using the MAST assay on three separate days using all combinations of antibiotics and organisms listed in “Bacterial strains and antimicrobials.” Each of these 1800 images was evaluated using our ConvNet-based pipeline (FIG. 24), and the MIC’s for the 180 dilution series was calculated using the optimal parameter and thresholds determined in the “Optimization of MIC calls” experiments described above. Only a single dilution series (n = 10 images) was omitted from further analyses based on an autofocus failure which compromised curve fitting and MIC determination.

Log₂ differences between MAST and BMD MICs were used to evaluate precision and accuracy of MAST. Taking into account the inherent variability in BMD, log₂ differences of ±1 were considered in essential agreement and equivalent as defined by CLSI guidelines¹⁷. Each MAST result was also assigned a categorical interpretation based on CLSI cutoffs² and compared to the categorical result from BMD to determine categorical agreement.

### Results

The rate-limiting step in traditional AST readout is the threshold for bulk microbial growth detection, either by optical density determination (as in the Vitek2) or human visualization of bacterial growth (in reference AST methods). Therefore, the fastest phenotypic AST readout presumably should be approached by microscopic visualization of the effects of antimicrobials on the replication of individually resolved bacterial cells. The idea of MAST was born on this premise.

The technical requirements for MAST included the need to visualize microorganisms on a solidified microwell surface; the ability to apply doubling dilution series of any antimicrobials desired; the ability to dispense organisms consistently at desired concentrations; and acquisition and automated classification of images as “growth” or “inhibition” with subsequent MIC calls. Investigation of each MAST component is discussed in turn along with preliminary validation of the end-to-end platform concept.

Preparation of solid microwell surfaces in microtiter plates. For efficient automated microscopic observation, bacteria must be located in a reproducible 3D location within microwells. In traditional liquid media used for AST testing, cells are distributed randomly throughout the volume of fluid and highly mobile based on Brownian motion or intrinsic motility, and thus unsuitable for single cell imaging. Therefore, we considered use of a solid growth surface containing standard nutrient medium (CAMHB) for image acquisition. Requirements for the solidifying agent included: formation of surfaces in microwells with consistent height; absence of inhibitory effects on bacterial growth; and amenability to pipetting.

We first evaluated standard microbiological agar. However, the solution was difficult to pipette consistently due to rapid and practically irreversible solidification at <40° C. Next, we investigated gellan gum, an anionic polysaccharide alternative to agar;² polyacrylamide; and poloxamer 407, a hydrophilic, nonionic copolymer, as alternative solidifying agents. Gellan gum supported bacterial growth, but preparation of consistent microwell surfaces was not possible due to phase transition characteristics similar to traditional agar. Polyacrylamide surfaces were exceptionally easy to prepare, but proved inhibitory to bacteria.

By contrast, a solution of 15% poloxamer 407 was identified as an ideal solidifying agent. Aqueous solutions of poloxamer 407 are liquid at 4° C. allowing for facile pipetting of solutions kept on ice. Solidification occurs at ~20° C. and is thermally reversible, allowing for preparation of plates at room temperature followed by centrifugation at 4° C. to ensure substrate is evenly distributed on the bottom of wells. Prepared plates can be frozen indefinitely with no effect on the integrity of growth surfaces. Further, CAMHB solidified with poloxamer 407 was found to support growth of all common Gram-negative bacterial pathogens tested (data not shown).

Digital dispensing of bacteria. Use of solid surfaces contained within single wells of 384-well plates allows for multiple organism/antibiotic combinations to be tested in a single experiment. However, use of small wells presented a technical challenge in that: (1) organisms must be quantitatively delivered without disturbing the integrity of the growth surface; (2) volumes must be small (nL size) to avoid flooding of the surface leading to uneven distribution of organisms and associated requirement for drying; and (3) delivery must be spatially precise to allow efficient positioning of an automated microscope over fields containing organisms. Manual pipetting or pin transfer cannot satisfy these requirements. Therefore, we investigated bacterial cell dispensing using the HP D300 as an alternative method.

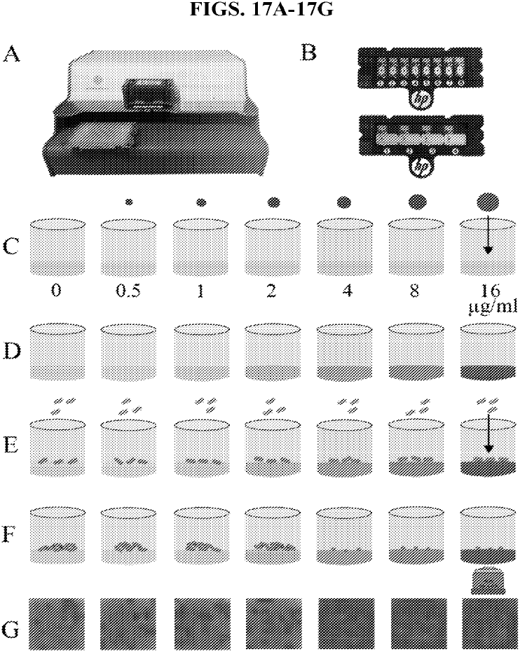

Bacterial cell dispensing is a novel application of inkjet printing technology. Of note, the HP D300 inkjet printer (FIG. 17A) used in our studies was designed to dispense droplet volumes ranging from 11 picoliters to 10 microliters per manufacturer’s specification⁸ from a single stock solution loaded into a reagent cassette (FIG. 17B). We previously used this technology to prepare doubling dilution series of antimicrobials in 384-well plates in liquid media,⁹ and in MAST used the same technique to apply antimicrobial dilutions to solid surfaces (FIGS. 17C-17D). We furthermore hypothesized that we could also use this technology to deliver any desired quantity of microorganism into microwells from a single bacterial suspension by choosing the appropriate droplet dispense size (FIG. 17E).

Therefore, we evaluated the ability of the HP D300 to dispense bacteria using two complimentary techniques: First, we evaluated the ability of the HP D300 to quantitatively deliver bacteria to microwells by measuring the number of cells dispensed in a given volume through CFU determination. We performed this evaluation with 5 organisms representative of the most common Gram-negative pathogens (E. coli, K. pneumoniae, E. cloacae, P. aeruginosa, and A. baumannii) to discern whether unique species-specific physical properties might impact ability of cells to travel through the cassette channel, thus affecting dispense accuracy.

In linear dynamic range studies, we dispensed between 50 to 350 nL of a bacterial suspension and quantified the number of cells delivered to a well. We constructed a standard curve and found that dispense volume was predictive of CFU (average R² = 0.96), indicating precise and reproducible cell dispensing (see FIGS. 10A-10E). Experiments were repeated on three separate days, and the day-to-day coefficient of variation was, on average, 34%. For each dispense volume, precision was evaluated for three wells spanning a series of 96-wells. No significant difference in bacterial dispense numbers was detected across wells (ANOVA, p > 0.05).

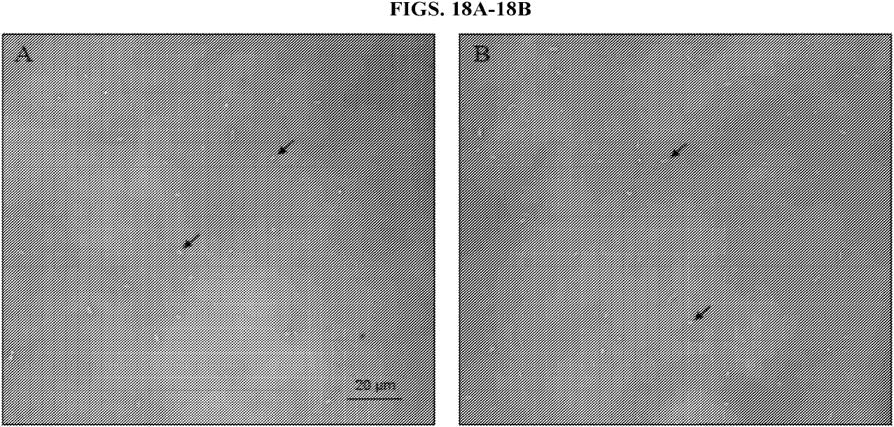

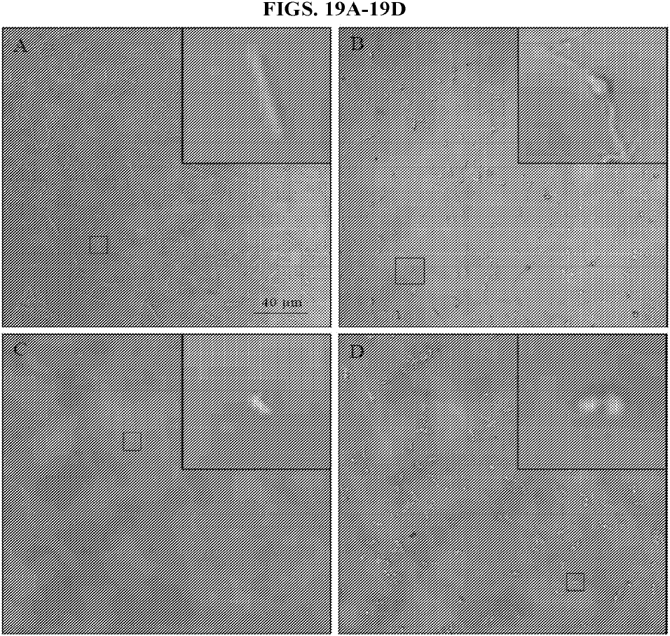

Second, after considering quantitative precision of dispensing, we evaluated spatial precision in a manner relevant to MAST. We dispensed bacteria into the center of wells and counted the number of digitally dispensed bacteria in a single central field using the Cell Observer microscope. This methodology allowed us to simultaneously evaluate spatial precision, quantitative precision, and reproducibility of digital dispensing. We found that 100% of wells imaged contained bacteria in the central field and that cells were well separated and evenly dispersed (FIGS. 18A-18B provide representative images). Average number of bacteria per field in the single selected central field across wells ranged, on average, from 150 to 260 (1.5 to 2.5 cells per 1000 µm²). Day-to-day coefficient of variation was 23% across all species tested. There was no significant difference in bacterial dispense numbers across all wells (ANOVA, p > 0.05).

During these studies, we found that bacteria could be clearly resolved in images using a long working distance 40X objective mounted on an inverted microscope, with brightfield optics, and standard polystyrene 384-well plates. Notably, use of optical quality 384-well plates with thin plastic film bottoms did not improve image resolution of bacteria growing on CAMHB-P surfaces. Further, phase-contrast and differential interference contrast (DIC) imaging of wells surprisingly led to inferior bacterial image resolution, perhaps because of optical properties and the thickness of plastic well bottom and poloxamer separating the objective and bacterial growth surface (total working distance approximately 2.9 mm).

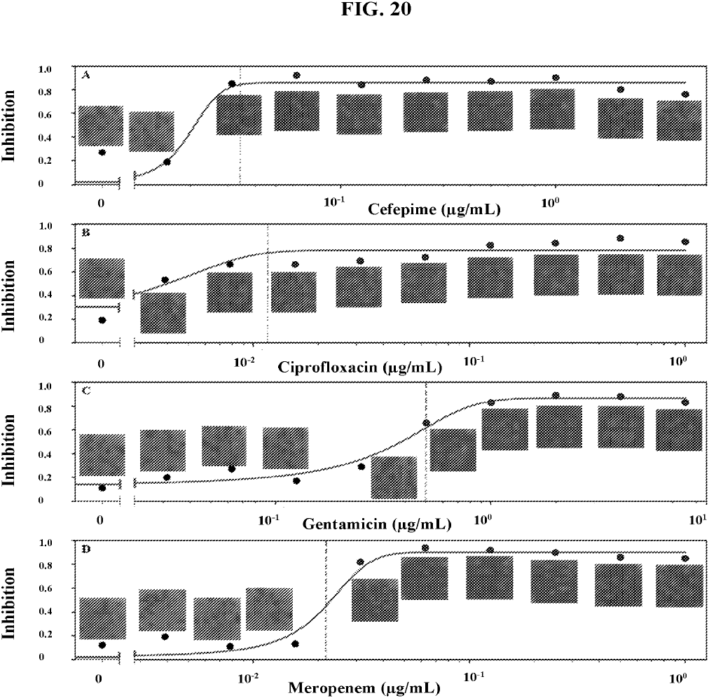

Microscopy-based antimicrobial susceptibility testing (MAST). We then combined bacterial and antibiotic dispensing with automated microscopy to perform microscale, “agar dilution” AST assays inside wells of a 384-well plate. Here, bacterial growth was monitored by automated microscopy (FIG. 17F) and images were classified as “growth” or “inhibition” using a machine learning algorithm to determine the minimal inhibitory concentration. For proof-of-concept studies, we chose to use four different antibiotics with clinical utility against Gram-negative bacteria. Each of these drugs was also specifically chosen based on unique and well-defined alterations in cell morphology at their MIC, all of which were observed using the MAST assay.¹³⁻¹⁵ Exposure to ciprofloxacin, a fluoroquinolone class, DNA gyrase inhibitor, resulted in elongated cells that failed to divide (FIG. 19A). Exposure to cefepime, a cephalosporin β-lactam-based cell wall synthesis inhibitor, resulted in filamented bacteria with a pronounced central bulge (FIG. 19B). Gentamicin, an aminoglycoside-based protein synthesis inhibitor, blocked cell growth, but did not appreciably alter morphology (FIG. 19C). Exposure to meropenem, a carbapenem β-lactam-based cell wall synthesis inhibitor, resulted in rounded sphereoplasts (FIG. 19D). We presumed that this range of cell morphologies would appropriately challenge automated image classification methods discussed in subsequent sections.

Image Classification by Deep Learning. To call MICs, at least one image field must be analyzed per microwell in a doubling dilution series. With typical dilution series (which may include up to 10 doubling dilutions) and desire to test multiple drugs against multiple organisms, manual inspection and classification of the resultant large image sets would be time prohibitive and inconsistent with future clinical translation. Therefore, we considered using machine-learning for automated classification of MAST images.

Specifically, we examined the ability of a deep convolutional neural network (ConvNet) to classify images for accurate determination of MIC values following only a two hour incubation period. Importantly, this accelerated time window contrasts with the 16-20 hour incubation period required for reference MIC determinations and similar extended incubation periods required for commercial AST methods. ConvNets excel at image recognition, and have recently achieved human-level performance on large-scale image classification tasks (He et al., ICCV2015). This approach takes a raw image as input and learns to extract relevant features for the particular task at hand. In other words, it does not rely on pre-conceived human biases regarding what features are likely important for optimal discrimination, but rather simultaneously learns which features to extract and how to combine them to achieve accurate classification, based on optimizing its output relative to a labeled training set.

The network was trained on a set of 3,202 full images (1024x1024 px) collected in three independent experiments in an attempt to capture the totality of biological and technical variability in the MAST assay and produce an algorithm that will generalize to a diverse set of conditions. After training, the network reached a peak classification accuracy of 90% on the held out validation set at the per-image-crop (220x220 px) level with the inhibition probability cutoff arbitrarily defined as 0.5. After training, the network reached a peak classification accuracy of 90% on the test set at the per-image-crop (1/50th of an image) level with the inhibition probability cutoff arbitrarily defined as 0.5. After mapping image crops back to whole images, regions representing growth or inhibition were manually inspected and found to correspond to expected growth/no growth morphologies (data not shown).

Optimization of MIC calls. To determine MICs, each image, which corresponds to a specific antimicrobial dilution, must be classified as growth or inhibition. Our image classification algorithm first evaluated 64 non-overlapping crops from of each image and determined mean inhibition probability, median inhibition probability, and number of crops with inhibition probability above 0.5. However, it was unknown which of these parameters and what threshold cutoff for each parameter would result in the most accurate MIC calls. Furthermore, we noted that absolute values of these parameters varied by approximately 20% on a day-to-day basis, suggesting need for a self-normalizing algorithm that would be robust to these differences. Specifically, effects of biological and technical variability were mitigated by modeling the results for each parameter (median inhibition probability, mean inhibition probability, and number of crops with inhibition probability > 0.5) across each dilution series as a sigmoid curve defined by the following equation (1): Eq (1). Sigmoidal curve function used to fit ConvNet output.

$$y = \frac{1}{c + {e}^{- k \left(x - {x}_{0}\right)}}$$

where y = inhibition probability; x = log (antimicrobial); e = constant (i.e., 2.7, base of natural log); and c, k, x₀ = variables optimized during curve fitting.

We collected an optimization dataset for the purpose of determining the best way to evaluate the ConvNet output. We then iteratively evaluated all possible discrimination thresholds normalized to each sigmoid curve for each antibiotic/organism/parameter for correct MIC prediction. Although the ConvNet was trained to predict growth/inhibition for individual images, here we evaluated and optimized performance against reference BMD MIC values determined for each dilution series.

Upon optimization of the discriminatory threshold for each parameter for all antibiotic/organism combinations, we identified the proportion of image crops with inhibition probability >0.5 as the optimal parameter for MIC determination. Importantly, we confirmed our prediction that a sigmoid curve would robustly model our data (average R² = 0.93).

Proof-of-principle, end-to-end testing of MAST Assay. To evaluate the “end-to-end” MAST assay, we collected a large verification dataset which was independent of both the training and optimization datasets. We then compared performance of our optimized MAST assay (the test method), that included only a two hour growth incubation, to BMD (the reference method), that used a standard 16-20 hour growth incubation. Sigmoidal curve fitting was equally robust in this phase (average R² = 0.91, FIG. 20).

Notably, MAST MIC essential agreement (±1 two-fold dilution) was 95.8% compared BMD. 99.4% of MIC results were within ±2 two-fold dilutions of modal reference BMD results (Table 8). MIC determinations for each antibiotic considered separately were similarly accurate and ranged from 93.3-100%. Notably, a 100% MIC accuracy was found for cefepime despite this antibiotic presenting a substantial challenge to the classification algorithm due to filamentation of cells near the MIC (FIG. 19B). In addition, 100% of results for E. coli ATCC 25922, a CLSI recommended control strain, were within ±1 dilution of BMD. Furthermore, categorical agreement (interpretation of organisms as susceptible, intermediate, or resistant) for the susceptible strain set between MAST and BMD was 98.3% (Table 8), indicating a very low level of false resistance (major errors).

TABLE 8

Evaluation of MAST Accuracy.

| Antimicrobial | Number of measurements with Log₂ difference from reference MIC^{a} | | | | | | Essential Agreement % (CI) | Categorical Agreement % (CI) |
|---|---|---|---|---|---|---|---|---|
| | -2 | -1 | 0 | 1 | 2 | -3 | | |
| Ciprofloxacin | 0 | 7 | 19 | 16 | 1 | 1 | 95.5 (84.5-98.7) | 100 (90.6-100) |
| Cefepime | 0 | 8 | 19 | 6 | 0 | 0 | 100 (89.3-100) | 93.3 (84.3-99.4) |
| Gentamicin | 3 | 9 | 16 | 17 | 0 | 0 | 93.3 (81.4-97.8) | 100 (92-100) |
| Meropenem | 1 | 7 | 14 | 21 | 1 | 0 | 95.5 (84.5-98.7) | 100 (92.1-100) |
| Total n (%, CI)^{b} | 4 (2.4) | 31 (18.7) | 68 (41) | 60 (36.1) | 2 (1.2) | 1 (0.6) | 95.8 (91.5-98) | 98.3 (95.2-99.4) |

“Reference MIC derived from broth microdilution
^{b}CI = 95% confidence interval

### Discussion

Extensive antimicrobial resistance amongst Gram-negative pathogens reduces reliability of empiric antimicrobial therapy, underscoring the critical importance of timely AST in the clinical microbiology laboratory to guide therapy. Importantly, delay in institution of active therapy is correlated with increased mortality during serious infection.⁷ However, laboratory tests in current use require 8-24 hours for results.

Rapid AST systems are a potential solution to address this issue and may be approached broadly through two pathways: phenotypic testing or genotypic testing. Genotypic assays call resistance based on presence of specific resistance elements. However, such assays are limited to evaluating known resistance determinants and are typically unable to determine exact MICs and thereby direct therapy based on known pharmacodynamic relationships. Therefore genotypic methods lack sensitivity and specificity.

In contrast, rapid phenotypic assays are more content rich as they have the capability of determining exact MICs. Furthermore, well established and validated pharmacodynamic relationships between drug exposure in vivo and the MIC are predictive of response to therapy.¹⁹ Accordingly, CLSI guidelines recognize phenotypic testing as the current methodology for determining susceptibility.² For Gram-negative organisms in particular, guidelines have moved away from detecting or inferring the presence of specific resistance elements to guide therapy.²⁰

We therefore developed and performed preliminary validation of a rapid phenotypic AST method based on observation of individual cells. Microscopic AST methods are faced with significant logistical and technical challenges, one of which is the immobilization of cells for observation. In most existing rapid AST instruments, this is accomplished by suspension of cells in a matrix or restriction of cells to a microfluidic channel.²¹ These procedures are complex and have no predicate in current clinical microbiology practice.

In contrast, the MAST assay immobilizes bacteria by dispensing cells on a solid microwell surfaces analogous to 1000-fold miniaturized agar plates, and in essence, replicates the existing agar dilution reference method.¹ Using inkjet technology, inoculation of an entire assay plate (up to 240 wells) can be accomplished quickly (<1 minute) in an operator-independent manner with a single pipetting step. The steps required for MAST assay performance (including preparation of bacterial suspensions) are similar to those for currently used AST systems and could be performed by a technologist without additional training.

Clinical automated AST systems typically include only a fixed and limited set of antimicrobials; furthermore, only a limited number of antimicrobial dilutions are tested per drug.³ In contrast, MAST allows preparation of plates dynamically using any number of antimicrobials with dilution series of any size in a high-density 384-well format. Similar to bacterial dispensing, an antimicrobial doubling dilution series can be prepared very quickly (<10 seconds) with a single pipetting step to load antimicrobial stock solution into a dispensing cassette as we described previously.⁹ Flexibility to prepare any doubling dilution series at will allows for testing antimicrobials at concentrations relevant to multiple species that may have different breakpoints on a single microplate.² Concentrations can easily be tailored to include changes in breakpoints that are recommended by agencies such as CLSI to accommodate evolving understanding of the relationships between MIC and clinical outcome.⁴ Ability to test antimicrobials at low concentrations ensures that standard quality control organism MIC ranges are on-scale so that assay performance can be appropriately monitored, something that is often not possible with current clinical systems which only include dilution series bracketing clinical breakpoints.^{2,}⁵

The large amount of image data generated by MAST led to the need for automated interpretative capabilities. Therefore, we developed a machine learning-based image classification algorithm. In this first stage conception of MAST, a ConvNet was trained based on human classification of a large set of training images collected after 2 hours of incubation. Importantly, images were collected from five organisms exposed to varying concentrations of four antibiotics over three separate days, such that the training set captured the biological and technical variability that might be seen in a clinical setting.

Ultimately, our image analysis pipeline contained three levels of image interpretation: (1) ConvNet image classification which returned per-image-crop probability of inhibition, a feature that in itself predicted grow status in image crops with > 90% accuracy. However, this output did not make a direct prediction regarding whole images. (2) Results from all image crops for each image were therefore pooled to provide image statistics (mean inhibition probability, median inhibition probability, and proportion of image crops with inhibition > 0.5). (3) Data from all images in a dilution series were then modeled as a sigmoid curve and classified based on thresholds that optimized accuracy of MIC calls.

This optimized algorithm was then applied to a new set of images and accuracy compared to the broth microdilution MIC. Importantly, accuracy achieved essential agreement metrics established by the FDA (Food and Drug Administration, Guidance for Industry and FDA. Class II Special Controls Guidance Document: Antimicrobial Susceptibility Test (AST) Systems. Food and Drug Administration: Rockville, MD, 2009) and standards in the field for evaluating new antimicrobial susceptibility testing methodology in comparison to the BMD reference methodology (Clark et al., Cumulative Techniques and Procedures in Clinical Microbiology 31A: Verification and Validation of Procedures in the Clinical Microbiology Laboratory. American Society for Microbiology: Washington, DC, 2009). Specifically, essential agreement was 95.8% (±1 dilution of the reference MIC) and exceeding the recommended 90% essential agreement threshold.

It should be noted that in this preliminary analysis, a generally susceptible strain set was tested. However, even with this caveat, we found only 3 interpretive errors. All of these interpretive errors were found with cefepime and P. aeruginosa ATCC 27853, which has a reference MIC only one doubling dilution below the categorical breakpoint². Moreover, the method appeared to perform equally well testing Enterobacteriaceae, P. aeruginosa, and A.baumannii, constituting the overwhelming majority of Gram-negative pathogens causing human infection. These organisms are also often associated with problematic drug resistance and therefore of high relevance for accelerated AST testing.²² Importantly, although our image analysis pipeline is not optimized for processing speed, a single 10-step doubling dilution series can still be evaluated in as little as 70 seconds through use of a dedicated graphics processing unit (GPU), allowing rapid image classification. Automated MIC calls based on sigmoidal modeling of ConvNet output are essentially instantaneous. In summary, in a first generation MAST platform we were able to achieve a high degree of accuracy after only two hours incubation.

Notably, typical rapid AST systems require custom made consumables.²¹ In contrast, the MAST assay relies entirely on off-the-shelf consumables including standard polystyrene 384-well plates, poloxamer-407, and antibiotic powder. When fully utilized, a MAST assay plate can accommodate testing of 4 pathogens against 6 antibiotics each, resulting in a consumable cost of <$3.50 per antibiotic/organism combination based on list price. Furthermore, all required instrumentation is commercially available. Importantly, MAST utilizes only a subset of features of the HP D300 and Zeiss Cell Observer microscope. Indeed for the latter, the only features used were standard light microscopy optics, a long working distance 40X lens, and a mechanical stage. Therefore, instrumentation could conceivably be simplified to further reduce costs during development of a next generation platform.

Here, we demonstrate proof of principle for a flexible AST platform that allows highly accurate MIC determination after only a 2 hour incubation. Importantly, the system allows determination of AST for any concentration of any antimicrobial at will in an operator-independent manner. This flexibility extends to incorporation of new antimicrobials that may not be available in commercial panels for years yet will likely be required to treat emerging multidrug-resistant pathogens. Further, MAST is a true MIC methodology that allows all relevant concentrations of antimicrobial to be tested without the need for MIC extrapolations that may not work reliably for resistant pathogens.²³ Taken together, proof-of-principle was obtained for the MAST platform that will address a critical unmet need for flexible, extremely rapid AST diagnostics.

### References for Example 6:

1. Clinical and Laboratory Standards Institute, Methods for dilution antimicrobial susceptibility tests for bacteria that grow aerobically; approved standard - tenth edition. CLSI document M07-A10. Clinical and Laboratory Standards Institute: Wayne, PA, 2015.
2. Clinical and Laboratory Standards Institute, Performance standards for antimicrobial susceptibility testing; twenty-seventh informational supplement. CLSI document M100-S27. Clinical and Laboratory Standards Institute: Wayne, PA, 2017.
3. Jorgensen, J. H.; Ferraro, M. J., Antimicrobial susceptibility testing: a review of general principles and contemporary practices. Clinical infectious diseases : an official publication of the Infectious Diseases Society of America 2009, 49 (11), 1749-55.
4. Humphries, R. M.; Hindler, J. A., Emerging Resistance, New Antimicrobial Agents ... but No Tests! The Challenge of Antimicrobial Susceptibility Testing in the Current US Regulatory Landscape. Clinical infectious diseases : an official publication of the Infectious Diseases Society of America 2016, 63 (1), 83-8.
5. Smith, K. P.; Brennan-Krohn, T.; Weir, S.; Kirby, J. E., Improved Accuracy of Cefepime Susceptibility Testing for Extended-Spectrum-Beta-Lactamase-Producing Enterobacteriaceae with an On-Demand Digital Dispensing Method. Journal of clinical microbiology 2017, 55 (2), 470-478.
6. Flynt, L. K.; Veve, M. P.; Samuel, L. P.; Tibbetts, R. J., Comparison of Etest to Broth Microdilution for Testing of Susceptibility of Pseudomonas aeruginosa to Ceftolozane-Tazobactam. Journal of clinical microbiology 2017, 55 (1), 334-335.
7. Schwaber, M. J.; Carmeli, Y, Mortality and delay in effective therapy associated with extended-spectrum beta-lactamase production in Enterobacteriaceae bacteraemia: a systematic review and meta-analysis. The Journal of antimicrobial chemotherapy 2007, 60 (5), 913-20.
8. Tecan Inc. Tecan D300e Digital Dispenser - Specification. Available at tecan.com/mandant/files/doc/526/BR _Tecan_D300e_Specifications_399178_Vl-0.pdf on the World Wide Web (accessed April 26).
9. Smith, K. P.; Kirby, J. E., Verification of an Automated, Digital Dispensing Platform for At-Will Broth Microdilution-Based Antimicrobial Susceptibility Testing. Journal of clinical microbiology 2016, 54 (9), 2288-93.
10. Hunter, J. D., Matplotlib: A 2D graphics environment. Computing in Science and Engineering 2007, 9 (3), 90-95.
11. van der Walt, S., Colbert, S.C., Varoquaux G., The NumPy Array: A Structure for Efficient Numerical Computation. Computing in Science and Engineering 2011, 13 (2), 22-30.
12. Schindelin, J.; Rueden, C. T.; Hiner, M. C.; Eliceiri, K. W., The ImageJ ecosystem: An open platform for biomedical image analysis. Molecular reproduction and development 2015, 82 (7-8), 518-29.
13. Pucci, M. J.; Boice-Sowek, J.; Kessler, R. E.; Dougherty, T. J., Comparison of cefepime, cefpirome, and cefaclidine binding affinities for penicillin-binding proteins in Escherichia coli K-12 and Pseudomonas aeruginosa SC8329. Antimicrobial agents and chemotherapy 1991, 35 (11), 2312-7.
14. Mason, D. J.; Power, E. G.; Talsania, H.; Phillips, I.; Gant, V. A., Antibacterial action of ciprofloxacin. Antimicrobial agents and chemotherapy 1995, 39 (12), 2752-8.
15. Horii, T.; Kobayashi, M.; Sato, K.; Ichiyama, S.; Ohta, M., An in-vitro study of carbapenem-induced morphological changes and endotoxin release in clinical isolates of gram-negative bacilli. The Journal of antimicrobial chemotherapy 1998, 41 (4), 435-42.
16. Simonyan, K.; Zisserman, A., Very deep convolutional networks for large-scale image recognition. Conference paper at ICLR 2015 2015, https://arxiv.org/pdf/1409.1556v6.pdf.
17. Clinical and Laboratory Standards Institute, Verification of Commercial Microbial Identification and Antimicrobial Susceptibility Testing Systems. CLSI document M52. Clinical and Laboratory Standards Institute: Wayne, PA, 2015.
18. LeCun, Y; Bengio, Y; Hinton, G., Deep learning. Nature 2015, 521 (7553), 436-44.
19. Onufrak, N. J.; Forrest, A.; Gonzalez, D., Pharmacokinetic and Pharmacodynamic Principles of Anti-infective Dosing. Clinical therapeutics 2016, 38 (9), 1930-47.
20. Clinical and Laboratory Standards Institute, Performance standards for antimicrobial susceptibility testing; nineteenth informational supplement. CLSI document M100-S19. Clinical and Laboratory Standards Institute: Wayne, PA, 2009.
21. Pulido, M. R.; Garcia-Quintanilla, M.; Martin-Pena, R.; Cisneros, J. M.; McConnell, M. J., Progress on the development of rapid methods for antimicrobial susceptibility testing. The Journal of antimicrobial chemotherapy 2013, 68 (12), 2710-7.
22. Boucher, H. W.; Talbot, G. H.; Bradley, J. S.; Edwards, J. E.; Gilbert, D.; Rice, L. B.; Scheld, M.; Spellberg, B.; Bartlett, J., Bad bugs, no drugs: no ESKAPE! An update from the Infectious Diseases Society of America. Clinical infectious diseases: an official publication of the Infectious Diseases Society of America 2009, 48 (1), 1-12.
23. Smith, K. P.; Kirby, J. E., How inkjet printing technology can defeat multidrug-resistant pathogens. Future microbiology 2016, 11, 1375-1377.

The present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

What is claimed is:

1. An automated system for microscopy-based antimicrobial susceptibility testing, the system comprising
a dispensing unit configured for automated dispensing of one or more compositions to one or more locations on a well plate;
a communication module configured to communicate with a data storage module comprising antimicrobial susceptibility protocol information;
a programmable controller configured to control an operation of the dispensing unit based on protocol information received from the data storage module via the communication module; and
a microscopy system for automated detection of antimicrobial susceptibility.

2. The automated system of claim 1, wherein the microscopy system is integrated with at least one of the dispensing unit, the communication module, and the programmable controller.

3. (canceled)

4. The automated system of claim 1, wherein the dispensing unit comprises
a first cassette configured to store and digitally dispense a suspension of at least one kind of cell or microorganism in a culture medium to one or more locations on the well plate; and
a second cassette configured to store and digitally dispense an antimicrobial agent to one or more locations for automated dispensing to one or more locations on the well plate.

5. The automated system of claim 4, wherein the at least one kind of cell or microorganism is selected from the group consisting of a prokaryotic cell, eukaryotic cell, bacterial cell, animal cell, fungus cell, insect cell, plant cell, virus, virus-containing host cell, and archaebacterial cell.

6. (canceled)

7. (canceled)

8. The automated system of claim 1, wherein the dispensing unit comprises
a first cassette configured to store a culture medium for automated dispensing to one or more locations on a well plate, wherein the culture medium comprises a biocompatible solidifying agent; whereby, upon dispensation to the one or more locations, the culture medium solidifies to form a solid or semi-solid culture substrate;
a second cassette configured to store and digitally dispense an antimicrobial agent to the one or more locations; and
a third cassette configured to store and digitally dispense a suspension of at least one kind of cell or microorganism in a culture medium to the one or more locations.

9. The automated system of claim 8, wherein the controller is programmed to dispense a predetermined quantity of culture medium from the first cassette, a predetermined quantity of antimicrobial agent from the second cassette, or a predetermined quantity of cell of interest from the third cassette.

10-12. (canceled)

13.

14. The automated system of claim 8, wherein the at least one kind of cell or microorganism is selected from the group consisting of a prokaryotic cell, eukaryotic cell, bacterial cell, animal cell, fungus cell, insect cell, plant cell, virus, virus-containing host cell, and archaebacterial cell.

15. (canceled)

16. An The automated system of claim 1, the system further comprising a microscopy system for automated detection of antimicrobial susceptibility.

17. The automated system of claim 16, wherein the microscopy system for automated detection of antimicrobial susceptibility is configured to obtain images of one or more locations on the well plate.

18. The automated system of claim 17, further comprising a programmable computing system configured for analysis of images obtained by the microscopy imaging system, wherein the programmable computing system and the microscopy imaging systems are in communication with each other.

19. The automated system of claim 18, wherein the programmable computing system comprises at least one of a data acquisition module, a processing module, and an analysis module.

20. The automated system of claim 18, wherein the programmable computing system is configured to analyze the images using a convolutional neural network trained to predict growth or inhibition for individual images.

21-27. (canceled)

28. An The automated system of claim 1, the system further comprising a means for detecting antimicrobial susceptibility.

29. The automated system of claim 28, wherein the means for detecting antimicrobial susceptibility is a means for spectrophotometric detection, microscopic detection, or fluorescence-based detection.

30. The automated system of claim 28, wherein the means for detecting antimicrobial susceptibility comprises a microscopy imaging system configured to obtain images of one or more locations on the well plate.

31. (canceled)

32. (canceled)

33. (canceled)

34. The automated system of claim 28, wherein the means for detecting antimicrobial susceptibility is integrated with at least one of the dispensing unit, the communication module, and the programmable controller.

35. The automated system of claim 28, further comprising a digital dispenser apparatus configured to dispense apportioned picoliter to microliter volumes of one or more compositions to one or more locations on the well plate.

36. The automated system of claim 28, wherein the dispensing unit comprises
a first cassette configured to store and digitally dispense a suspension of at least one kind of cell or microorganism in a culture medium to one or more locations on the well plate; and
a second cassette configured to store and digitally dispense an antimicrobial agent to one or more locations for automated dispensing to one or more locations on the well plate.

37. A method of using a digital dispenser apparatus for antimicrobial synergy testing, the method comprising
(a) manually pipetting two or more compositions into a digital dispenser apparatus configured to dispense apportioned picoliter to microliter volumes of each composition to one or more locations on a well plate, wherein each of the two or more compositions comprises a different antimicrobial agent;
(b) manually pipetting a suspension of at least one kind of cell or microorganism in a culture medium into a digital dispenser apparatus configured to dispense apportioned picoliter to microliter volumes of the suspension to the one or more locations on a well plate of step (a); and
(c) detecting susceptibility of the at least one kind of cell or microorganism to the microbial agents of the two or more compositions;
(d) calculating a minimal inhibitory concentration (MIC) for each antimicrobial agent and calculating a fractional inhibitory concentration index (FIC_{I}), wherein the antimicrobial agents exhibit synergy where the FIC_{I} is ≤0.5.
